(19) United States
(12) Patent Application Publication
(10) Pub. No.: US 2026/0306098 A1
(43) Pub. Date: Oct. 8, 2026

(54) ADAPTER ASSEMBLIES FOR SURGICAL DEVICE

(71) Applicant: Covidien LP, Mansfield, MA (US)

(72) Inventors: Kenneth M. CAPPOLA, Monroe, CT (US); Pawel ABRAMEK, Berlin, CT (US); Kenneth H. WHITFIELD, North Haven, CT (US); Justin P. WILLIAMS, Southbury, CT (US); Roanit A. FERNANDES, Bethany, CT (US); Jacob C. BARIL, Norwalk, CT (US); Anthony D. CALDERONI, Bristol, CT (US); John J. HRYB, Southington, CT (US); Vincent LAFRAGOLA, JR., Killingworth, CT (US)

(21) Appl. No.: 19/478,906

(22) PCT Filed: Apr. 23, 2024
(86) PCT No.: PCT/US2024/025830

§ 371 Date: Oct. 27, 2025

Related U.S. Application Data
(60) Provisional application No. 63/462,302, filed on Apr. 27, 2023.

(51) Int. Cl. A61B 17/072

(52) U.S. Cl. CPC A61B 17/07207; A61B 2017/07257; A61B 2017/07271; A61B 2017/07278; A61B 2017/07285

(57) ABSTRACT

An adapter assembly is configured to engage a surgical device. The adapter assembly is configured to convert rotation of at least one motor of the surgical device into rotation, articulation, and/or actuation of an end effector assembly of the adapter assembly. The adapter assembly includes at least one of an articulation locking assembly, a spring-loaded articulation assembly, an articulation control mechanism, a sled detection mechanism, a knife detection mechanism, and/or an electrical assembly.

### CROSS-REFERENCE TO RELATED APPLICATIONS

The present application claims the benefit of and priority to U.S. Provisional Patent Application Ser. No. 63/462,302, filed on Apr. 27, 2023, the entire content of which is incorporated herein by reference.

### BACKGROUND

The present disclosure relates to adapter assemblies for a surgical device. More specifically, the present disclosure relates to adapter assemblies for engaging an electromechanical surgical device and for performing surgical procedures.

One type of surgical device is a linear clamping, cutting and stapling device. Such a device may be employed in a surgical procedure to resect a cancerous or anomalous tissue from a gastro-intestinal tract. Conventional linear clamping, cutting and stapling instruments include a pistol grip-styled structure having an elongated shaft and distal portion. The distal portion includes a pair of scissors-styled gripping elements, which clamp the open ends of the colon closed, for instance. In this device, one of the two scissors-styled gripping elements, such as the anvil portion, moves or pivots relative to the overall structure, whereas the other gripping element remains fixed relative to the overall structure. The actuation of this scissoring device (the pivoting of the anvil portion) may be controlled by a grip trigger maintained in the handle.

In addition to the scissoring device, the distal portion also includes a stapling mechanism. The fixed gripping element of the scissoring mechanism includes a staple cartridge receiving region and a mechanism for driving the staples up through the clamped end of the tissue against the anvil portion, thereby sealing the previously opened end. The scissoring elements may be integrally formed with the elongated shaft or may be detachable such that various scissoring and stapling elements may be interchangeable.

A number of surgical device manufacturers have developed product lines with proprietary powered drive systems for operating and/or manipulating the surgical device. In many instances the surgical devices include a powered handle assembly, which is reusable, and a disposable adapter assembly or the like that is selectively connected to the powered handle assembly prior to use and then disconnected from the powered handle assembly following use in order to be disposed of or in some instances sterilized for re-use.

### SUMMARY

This disclosure relates to an adapter assembly for use with a surgical device. The adapter assembly includes an outer tube, an end effector assembly, and an articulation assembly. The outer tube defines a longitudinal axis. The end effector assembly is disposed adjacent a distal end of the outer tube. The articulation assembly is configured to convert rotation of a first shaft of the surgical device into movement of the end effector assembly between a first position where the end effector assembly is disposed along the longitudinal axis, and a second position where the end effector assembly is disposed at an angle relative to the longitudinal axis.

The articulation assembly includes a drive tube, a first pin, a second pin, a first cam, a second cam, a first articulation member, and a second articulation member. The drive tube is rotatable about the longitudinal axis relative to the outer tube. The first pin is engaged with the drive tube and extends radially inward from the drive tube. The second pin is engaged with the drive tube and extends radially inward from the drive tube. The first cam defines a first thread engaged with the first pin. The first thread encircles the first cam in a first direction. The second cam defines a second thread engaged with the second pin. The second thread encircles the second cam in a second direction. The second direction is opposite from the first direction. A proximal portion of the first articulation member is disposed in mechanical cooperation with the first cam, and a distal portion of the first articulation member disposed in mechanical cooperation with the end effector assembly. A proximal portion of the second articulation member is disposed in mechanical cooperation with the second cam, and a distal portion of the second articulation member disposed in mechanical cooperation with the end effector assembly. Rotation of the drive tube in a first direction about the longitudinal axis relative to the outer tube causes the first articulation member to move distally relative to the outer tube and causes the second articulation member to move proximally relative to the outer tube.

In disclosed embodiments, rotation of the drive tube in a second direction about the longitudinal axis relative to the outer tube causes the first articulation member to move proximally relative to the outer tube and causes the second articulation member to move distally relative to the outer tube.

In disclosed embodiments, the first articulation member is rotatable about the longitudinal axis relative to the first cam. In embodiments, the second articulation member is rotatable about the longitudinal axis relative to the second cam.

In disclosed embodiments, at least a portion of the first cam is movable into a channel defined by the second cam.

In disclosed embodiments, the first cam and the second cam are rotatable relative to the drive tube.

In disclosed embodiments, the drive tube is fixed from longitudinal movement relative to the outer tube.

In disclosed embodiments, the adapter assembly includes a firing rod assembly coupled to the end effector assembly. Distal movement of the firing rod assembly is configured to cause a first jaw member of the end effector assembly to move toward a second jaw member of the end effector assembly. In embodiments, the adapter assembly includes an actuation assembly configured to convert rotation of a second shaft of the surgical device into longitudinal movement of the firing rod assembly. The actuation assembly includes a drive shaft and a coupling nut. The drive shaft is disposed along the longitudinal axis and is rotatable about the longitudinal axis relative to the outer tube. The drive shaft includes a threaded portion. The coupling nut is threadedly engaged with the threaded portion of the drive shaft. Rotation of the drive shaft about the longitudinal axis causes longitudinal movement of the coupling nut relative to the outer tube.

In disclosed embodiments, a proximal portion of the firing rod assembly is mechanically engaged with the coupling nut.

In disclosed embodiments, the firing rod assembly is rotatable about the longitudinal axis relative to the coupling nut.

In disclosed embodiments, the coupling nut is positioned closer to the longitudinal axis than the first cam and the second cam.

In disclosed embodiments, the first articulation member is positioned closer to the longitudinal axis than the second articulation member.

In disclosed embodiments, the adapter assembly includes a rotation assembly configured to convert rotation of a third shaft of the surgical device into rotation of the end effector assembly about the longitudinal axis relative to the drive tube.

This disclose also relates to an actuation assembly for use with a surgical device. The actuation assembly includes an I-beam having a first leg, a second leg, a body portion, and a pin. The first leg is configured to travel at least partially through an anvil assembly of the surgical device. The second leg is configured to travel at least partially through a cartridge assembly of the surgical device. The body portion interconnects the first leg and the second leg. The pin extends between the fist leg and the second leg, and is rotatable relative to the first leg and the second leg.

In disclosed embodiments, the pin extends at least partially through an aperture defined in the first leg and at least partially through an aperture defined in the second leg.

In disclosed embodiments, the pin is rotatable relative to the body portion.

In disclosed embodiments, the pin is movable toward the first leg.

In disclosed embodiments, the I-beam includes a blade disposed on a distal edge of the body portion.

In disclosed embodiments, the pin is disposed proximally of a proximal edge of the body portion. In embodiments a proximal edge of the first leg is disposed proximally of the pin. In embodiments, a proximal edge of the second leg is disposed proximally of the pin.

In disclosed embodiments, the actuation assembly includes a drive beam assembly coupled to the pin of the I-beam. In embodiments, the drive beam assembly includes a plurality of layers. In embodiments, a first layer of the plurality of layers of the drive beam assembly is affixed to the pin of the I-beam. In embodiments, a second layer of the plurality of layers of the drive beam assembly is affixed to the first layer of the plurality of layers of the drive beam assembly and is free from contact with the I-beam.

In embodiments including the drive beam assembly, the plurality of layers of the drive beam assembly includes a first layer, a second layer, and a third layer. The first layer is affixed to the second layer, the third layer is affixed to the second layer, the second layer is affixed to the I-beam, the first layer is free from contact with the I-beam, and the third layer is free from contact with the I-beam.

In disclosed embodiments, a distal edge of the drive beam assembly is disposed distally of a proximal edge of the first leg of the I-beam.

In disclosed embodiments, the drive beam assembly is movable toward the first leg of the I-beam.

In disclosed embodiments, the drive beam assembly is pivotable relative to the body portion of the I-beam.

The disclosure also relates to an articulation assembly for use with a surgical device. The articulation mechanism includes a first articulation member, a second articulation member, a first spring-loaded articulation mechanism, and a second spring-loaded articulation mechanism. The first articulation member is disposed in mechanical cooperation with an end effector assembly of the surgical device. The second articulation member is disposed in mechanical cooperation with the end effector assembly of the surgical device. The first spring-loaded articulation mechanism is disposed in mechanical cooperation with the first articulation member and with the second articulation member. The first spring-loaded articulation mechanism includes a proximal boss, a distal boss, and a first biasing member extending between the proximal boss and the distal boss. The second spring-loaded articulation mechanism is disposed in mechanical cooperation with the first articulation member and with the second articulation member. The second spring-loaded articulation mechanism includes a proximal boss, a distal boss, and a second biasing member extending between the proximal boss and the distal boss. Distal movement of the first articulation member relative to the second articulation member results in compression of the first biasing member and compression of the second biasing member.

In disclosed embodiments, distal movement of the second articulation member relative to the first articulation member results in compression of the first biasing member and compression of the second biasing member.

In disclosed embodiments, the proximal boss of the first spring-loaded articulation mechanism is longitudinally movable toward and away from the end effector assembly of the surgical device. In embodiments, the distal boss of the first spring-loaded articulation mechanism is longitudinally movable toward and away from the end effector assembly of the surgical device.

In disclosed embodiments, the first articulation member defines a first recess having a proximal face and a distal face, and the second articulation member defines a second recess having a proximal face and a distal face. In embodiments, the proximal boss of the first spring-loaded articulation mechanism is positioned in contact with at least one of the proximal face of the recess of the first articulation member or the proximal face of the recess of the second articulation member. In embodiments, the distal boss of the first spring-loaded articulation mechanism is positioned in contact with at least one of the distal face of the recess of the first articulation member or the distal face of the recess of the second articulation member.

In disclosed embodiments, proximal movement of the first articulation member relative to the second articulation member results in the distal face of the first recess urging the distal boss of the first spring-loaded articulation mechanism proximally. In embodiments, distal movement of the second articulation member relative to the first articulation member results in the proximal face of the second recess urging the proximal boss of the first spring-loaded articulation mechanism distally. In embodiments, when the distal face of the first recess urges the distal boss of the first spring-loaded articulation mechanism proximally, the proximal face of the second recess urges the proximal boss of the first spring-loaded articulation mechanism distally at the same time.

The disclosure also relates to an articulation assembly for use with a surgical device. The articulation assembly includes a first articulation member, a second articulation member, a first spring-loaded articulation mechanism, and a second spring-loaded articulation mechanism. The first articulation member is disposed in mechanical cooperation with an end effector assembly of the surgical device. The second articulation member is disposed in mechanical cooperation with the end effector assembly of the surgical device. The first spring-loaded articulation mechanism is disposed in mechanical cooperation with the first articulation member, and includes a first distal linkage, a first stop member, and a first biasing member. The first biasing member is disposed between the first distal linkage and the first stop member. The second spring-loaded articulation mechanism is disposed in mechanical cooperation with the second articulation member, and includes a second distal linkage, a second stop member, and a second biasing member. The second biasing member is disposed between the second distal linkage and the second stop member. Distal movement of the first articulation member relative to the first stop member results in the first biasing member moving toward an expanded orientation.

In disclosed embodiments, proximal movement of the second articulation member relative to the second stop member results in the second biasing member moving toward a compressed orientation. In embodiments, the first biasing member is configured to move toward the expanded orientation at the same time as the second biasing member moves toward the compressed orientation.

In disclosed embodiments, the first distal linkage is directly engaged with the first articulation member. In embodiments, the first distal linkage is fixed from longitudinal movement relative to the first articulation member. In embodiments, the second distal linkage is directly engaged with the second articulation member. In embodiments, the second distal linkage is fixed from longitudinal movement relative to the second articulation member.

The disclosure further relates to a surgical device, including an end effector having a surgical tool; an outer tube having a distal end, the end effector pivotably connected to the distal end of the outer tube; and an articulation assembly. The articulation assembly includes a first articulation member disposed in mechanical cooperation with the end effector assembly; a second articulation member disposed in mechanical cooperation with the end effector assembly; a first spring-loaded articulation mechanism disposed in mechanical cooperation with the first articulation member, the first spring-loaded articulation mechanism including a first distal linkage, a first stop member, and a first biasing member, the first biasing member disposed between the first distal linkage and the first stop member; and a second spring-loaded articulation mechanism disposed in mechanical cooperation with the second articulation member, the second spring-loaded articulation mechanism including a second distal linkage, a second stop member, and a second biasing member, the second biasing member disposed between the second distal linkage and the second stop member.

In operation, distal movement of the first articulation member relative to the first stop member results in the first biasing member moving toward an expanded orientation. Further, in operation, proximal movement of the second articulation member relative to the second stop member results in the second biasing member moving toward a compressed orientation.

In disclosed embodiments, the first biasing member is configured to move toward the expanded orientation at the same time as the second biasing member moves toward the compressed orientation.

The disclosure relates to an articulation locking assembly for use with a surgical device. The articulation locking assembly includes a firing rod assembly, a collet, and a first brake. The firing rod assembly defines a longitudinal axis. The collet is configured to engage the firing rod assembly, and includes a body portion, and a leg extending from the body portion. The first brake is configured to selectively engage a first articulation member of the surgical device. The collet is longitudinally movable relative to the first brake between a first position where the body portion is free from contact with the first brake, and a second position where the body portion is in contact with the first brake. When the collet is in the first position, the first articulation member of the surgical device is capable of moving longitudinally relative to the firing rod assembly, and when the collet is in the second position, the first articulation member of the surgical device is prevented from moving longitudinally relative to an outer tube of the surgical device.

In disclosed embodiments, when the collet is in the second position, the firing rod assembly is longitudinally movable relative to the body portion of the collet.

In disclosed embodiments, the body portion of the collet is cylindrical.

In disclosed embodiments, the first brake is movable relative to the firing rod assembly in a direction that is perpendicular to the longitudinal axis.

In disclosed embodiments, the articulation locking assembly includes a second brake configured to selectively engage a second articulation member of the surgical device.

In disclosed embodiments, when the collet is in the first position, the second articulation member of the surgical device is capable of moving longitudinally relative to the firing rod assembly, and when the collet is in the second position, the second articulation member of the surgical device is prevented from moving longitudinally relative to the outer tube of the surgical device.

In disclosed embodiments, the first brake includes a plurality of teeth configured to selectively mesh with a plurality of teeth on the first articulation member of the surgical device.

In disclosed embodiments, the first brake includes an inner portion and an outer portion. The inner portion is closer to the longitudinal axis than the outer portion, and the inner portion made from a different material than the outer portion.

In disclosed embodiments, the leg of the collet extends proximally from the body portion.

In disclosed embodiments, a proximal portion of the leg of the collet is biased toward the longitudinal axis.

In disclosed embodiments, a proximal end of the leg of the collet includes a tapered portion. In embodiments, the tapered portion of the collet is configured to selectively engage a finger or an annular finger of the firing rod assembly.

In disclosed embodiments, distal movement of the firing rod relative to the first brake moves the collet from the first position to the second position. In embodiments, proximal movement of the firing rod relative to the first brake moves the collet from the second position to the first position.

In disclosed embodiments, a proximal end of the leg of the collet is movable between a first position where the proximal end of the leg is at least partially within a groove of the firing rod assembly, and a second position where the proximal end of the leg is disposed proximally of the groove of the firing rod assembly.

The disclosure relates to an adapter assembly for use with a surgical device. The adapter assembly includes an outer tube, an end effector assembly, a pivot assembly, and an articulation control mechanism. The outer tube defines a longitudinal axis. The end effector assembly is disposed adjacent a distal end of the outer tube, and defines a second longitudinal axis. The end effector assembly is movable between a first position where the second longitudinal axis is aligned with the first longitudinal axis, and a second position where the second longitudinal axis is offset from the first longitudinal axis. The pivot assembly is disposed adjacent a proximal end of the end effector assembly. The articulation control mechanism includes a lock, a block, and a plurality of notches defined the pivot assembly. The lock is disposed in mechanical cooperation with the outer tube, and includes a body portion, a finger extending distally from the body portion, and a first leg extending proximally from the body portion. The block is mechanically engaged with the outer tube, and is configured to engage the first leg of the lock. The finger of the lock is movable from a proximal position where the finger is free from engagement with the pivot assembly to a distal position where the finger is engaged with at least one notch of the plurality of notches of the pivot assembly.

In disclosed embodiments, the adapter assembly includes a drive beam assembly disposed at least partially within the outer tube. Distal translation of the drive beam assembly is configured to effect a function of the end effector assembly.

In disclosed embodiments, distal translation of the drive beam assembly causes the finger of the lock to move from its proximal position to its distal position.

In disclosed embodiments, the first leg of the lock of the articulation control mechanism is configured to selectively engage the drive beam assembly.

In disclosed embodiments, a proximal portion of the first leg of the lock of the articulation control mechanism is biased away from the drive beam assembly.

In disclosed embodiments, the adapter assembly includes a foot disposed at a proximal end of the first leg of the lock of the articulation control mechanism. The foot extends at an angle from the first leg. In embodiments, the foot of the lock of the articulation control mechanism is movable between a first position where the foot is at least partially within a recess defined by the drive beam assembly, and a second position where the foot spaced from the recess defined by the drive beam assembly.

In disclosed embodiments, a distal end of the block includes a ramped surface configured to selectively engage the first leg of the lock of the articulation control mechanism.

In disclosed embodiments, the block is fixed from longitudinal movement relative to the outer tube.

In disclosed embodiments, the lock of the articulation control mechanism includes a second leg configured to slidingly engage the block. In embodiments, the second leg of the lock of the articulation control mechanism includes a foot configured to selectively engage a tab of the drive beam assembly.

In disclosed embodiments, wherein a predetermined amount of proximal movement of the drive beam assembly relative to the outer tube causes the tab of the drive beam assembly to engage the foot of the second leg and move the finger of the lock of the articulation control mechanism proximally relative to the outer tube from its distal position to its proximal position.

In disclosed embodiments, the block of the articulation control mechanism includes a proximal wall configured to selectively engage the foot of the second leg of the lock of the articulation control mechanism.

In disclosed embodiments, the block of the articulation control mechanism includes a proximal wall configured to contact the foot of the second leg of the lock of the articulation control mechanism when the drive beam assembly is in a proximal-most position.

In disclosed embodiments, the foot of the second leg of the lock of the articulation control mechanism is positioned proximally of the foot of the first leg of the lock of the articulation control mechanism.

The disclosure relates to an end effector assembly for use with a surgical device. The end effector assembly includes a first jaw member, a second jaw member, a knife, and a lockout spring. The first jaw member defines a recess. The second jaw member is pivotal relative to the first jaw member between an open position and an approximated position. The knife is configured for longitudinal translation relative to the second jaw member, and includes a finger. The lockout spring includes a base and a leg. The base is coupled to the first jaw member, and the leg is movable between a first position where the leg is spaced from the recess of the first jaw member and a second position where the leg is disposed at least partially within the recess of the first jaw member. The leg is biased to the first position. When the finger of the knife is longitudinally aligned with the leg of the lockout spring, movement of the second jaw member from the open position to the approximated position causes the finger of the knife to contact the leg of the lockout spring and move the leg from the first position to the second position.

In disclosed embodiments, an actuation mechanism of the surgical device is permitted to move distally past the leg of the lockout spring when the leg of the lockout spring is in the second position. In embodiments, the lockout spring physically prevents the actuation mechanism of the surgical device from moving distally past the leg of the lockout spring when the leg is in the first position.

In disclosed embodiments, the base of the lockout spring is disposed distally of the leg of the lockout spring.

In disclosed embodiments, the knife includes a blade configured to cut tissue. A distal edge of the blade is disposed proximally of a distal edge of the finger of the knife.

In disclosed embodiments, the recess in the first jaw member is “T”-shaped. In embodiments, the lockout spring is “T”-shaped.

In disclosed embodiments, the base of the lockout spring is affixed to the first jaw member.

The disclose also relates to an end effector assembly for use with a surgical device. The end effector assembly includes a first jaw member, a second jaw member, a sled, and a sled lockout. The second jaw member defines a longitudinal slot. At least one of the first jaw member or the second jaw member is pivotal relative to the other jaw member between an open position and an approximated position. The sled is configured for longitudinal translation relative to the second jaw member, and includes an arm having a finger. At least a portion of the arm is configured to translate within the longitudinal slot of the second jaw member. The sled lockout is disposed in engagement with the second jaw member, and includes a body portion and a camming tab extending proximally form the body portion. The body portion is movable between a first position where the body portion is a first distance from the longitudinal slot of the second jaw member and a second position where the body portion is a second distance from the longitudinal slot of the second jaw member. The first distance is smaller than the second distance. Distal translation of the sled relative to the second jaw member causes a portion of the finger of the sled to contact the camming tab of the sled lockout and move the sled lockout from its first position to its second position.

In disclosed embodiments, an actuation mechanism of the surgical device is permitted to move distally past the body portion of the sled lockout when the body portion of the sled lockout is in the second position. In embodiments, the sled lockout physically prevents the actuation mechanism of the surgical device from moving distally past the body portion of the sled lockout when the body portion of the sled lockout is in the first position.

In disclosed embodiments, the body portion of the sled lockout is biased into its first position.

In disclosed embodiments, the sled lockout is disposed in pivotal engagement with the second jaw member.

In disclosed embodiments, the sled includes a wedge portion configured to engage pushers. In embodiments, the arm of the sled is pivotal relative to the wedge portion of the sled.

### Electronics Design

The disclosure relates to an adapter assembly for use with a surgical device. The adapter assembly includes a knob housing, an outer tube, an end effector assembly, and an electrical assembly. The outer tube extends distally from the knob housing and defines a first longitudinal axis. The end effector assembly is disposed adjacent a distal end of the outer tube and defines a second longitudinal axis. The end effector is movable between a first position where the second longitudinal axis is aligned with the first longitudinal axis, and a second position where the second longitudinal axis is disposed at an angle relative to the first longitudinal axis. The electrical assembly is configured to relay information from the end effector assembly to the surgical device when the adapter assembly is engaged with the surgical device. The electrical assembly includes a proximal electrical assembly and a flexible cable assembly. At least a portion of the proximal electrical assembly is disposed within the knob housing. The flexible cable assembly extends distally from the proximal electrical assembly. At least a portion of the flexible cable assembly is disposed within the outer tube. The flexible cable assembly includes an intermediate portion and a distal portion. The intermediate portion is configured to apply a proximally-directed force on the distal portion.

In disclosed embodiments, the intermediate portion of the flexible cable assembly of the electrical assembly includes an extendable section of cable. In embodiments, the extendable section of cable includes a sinusoidal section.

In disclosed embodiments, the adapter assembly includes a biasing element configured to bias the intermediate portion of the flexible cable assembly proximally.

In disclosed embodiments, the proximal electrical assembly includes a service loop disposed within the knob housing. In embodiments, the knob housing is fixed from rotation about the first longitudinal axis relative to the outer tube. In embodiments, the service loop encircles the first longitudinal axis.

In disclosed embodiments, the proximal electrical assembly includes a strain gauge.

In disclosed embodiments, the proximal electrical assembly includes at least one circuit board. In embodiments, the intermediate portion of the flexible cable assembly is rotatable about the first longitudinal axis relative to the at least one circuit board.

In disclosed embodiments, the distal portion of the flexible cable assembly of the electrical assembly is configured to move proximally relative to the outer tube when the end effector assembly moves from its first position to its second position.

In disclosed embodiments, the electrical assembly includes an NFC (near field communication) assembly. At least a portion of the NFC assembly is disposed adjacent the distal portion of the flexible cable assembly.

In disclosed embodiments, the adapter assembly includes a cartridge configured to selectively engage a cartridge channel of the end effector assembly. In embodiments, the NFC assembly includes an NFC tag disposed on the cartridge, and an NFC reader disposed on the cartridge channel of the end effector assembly. dr

### BRIEF DESCRIPTION OF THE DRAWINGS

Embodiments of the present disclosure are described herein with reference to the accompanying drawings, wherein:

FIG. 1 is a perspective view of a surgical device and an adapter assembly coupled to the surgical device, in accordance with embodiments of the present disclosure;

FIG. 2 is a perspective view of the surgical device of FIG. 1;

FIG. 3 is a front perspective view, with parts separated, of the surgical device of FIGS. 1 and 2;

FIG. 4 is a rear perspective view, with parts separated, of the surgical device of FIGS. 1 and 2;

FIG. 5 is a perspective view illustrating insertion of a power-pack into an outer shell housing of the surgical device;

FIG. 6 is a perspective view illustrating the power-pack nested into the outer shell housing of the surgical device;

FIG. 7 is a side elevational view of the outer shell housing of the surgical device;

FIG. 8 is a bottom perspective view of the outer shell housing of the surgical device, and an insertion guide thereof;

FIG. 9 is an enlarged, bottom perspective view of the outer shell housing of the surgical device with the insertion guide separated therefrom;

FIG. 10 is a first perspective view of the insertion guide;

FIG. 11 is a second perspective view of the insertion guide;

FIG. 12 is a front, perspective view of the power-pack with an inner rear housing separated therefrom;

FIG. 13 is a rear, perspective view of the power-pack with the inner rear housing removed therefrom;

FIG. 14 is a perspective view of a power-pack core assembly of the power-pack;

FIG. 15 is a front, perspective view of a motor assembly and a control assembly of the power-pack core assembly of FIG. 14;

FIG. 16 is a rear, perspective view, with parts separated, of the motor assembly and the control assembly of FIG. 15;

FIG. 17 is a longitudinal, cross-sectional view of the surgical device of FIG. 2;

FIG. 18 is an enlarged view of the indicated area of detail of FIG. 17;

FIG. 19 is a cross-sectional view of the surgical device as taken through line 19-19 of FIG. 17;

FIG. 20 is a perspective view of the surgical device, the adapter device, and a cartridge shown detached from each other, in accordance with embodiments of the present disclosure;

FIG. 21 is a perspective view of the adapter assembly of FIG. 20;

FIG. 22 is a cross-section view of the adapter assembly taken along line 22-22 of FIG. 21;

FIG. 23 is an enlarged view of the area of detail indicated in FIG. 22;

FIG. 24 is an enlarged view of the area of detail indicated in FIG. 22;

FIG. 25 is a transverse cross-sectional view of the adapter assembly taken along line 25-25 in FIG. 23;

FIG. 26 is a perspective view of a proximal portion of the adapter assembly of FIG. 20 with portions omitted;

FIG. 27 is a perspective view of components of the adapter assembly of FIG. 20;

FIG. 28 is a perspective view of components of the adapter assembly of FIG. 20;

FIG. 29 is a perspective, assembly view of various components of the adapter assembly of FIG. 20;

FIG. 30 is an enlarged view of the area of detail indicated in FIG. 29;

FIG. 31 is a perspective view of a distal portion of the outer tube of the adapter assembly of FIG. 20;

FIG. 32 is a cross-section view of the adapter assembly taken along line 32-32 in FIG. 31;

FIG. 33 is a perspective, assembly view of portions of the outer tube of the adapter assembly of FIG. 20 including articulation assemblies;

FIG. 34 is a cross-section view of a distal portion of the outer tube of the adapter assembly of FIG. 20 including adapter assemblies and showing the end effector assembly in a first, non-articulated position;

FIG. 35 is an enlarged view of the area of detail indicated in FIG. 34;

FIG. 36 is an enlarged view of the area of detail indicated in FIG. 34;

FIG. 37 is a cross-section view of a distal portion of the outer tube of the adapter assembly of FIG. 20 including adapter assemblies and showing the end effector assembly in a second, articulated position;

FIG. 38 is an enlarged view of the area of detail indicated in FIG. 37;

FIG. 39 is an enlarged view of the area of detail indicated in FIG. 37;

FIG. 40 is a perspective view of an articulation locking assembly, I-beam assembly and portions of a firing rod assembly of the adapter assembly of FIG. 20;

FIG. 41 is a perspective, assembly view of the articulation locking assembly and I-beam assembly of FIG. 40;

FIG. 42 is an enlarged view of the area of detail indicated in FIG. 41;

FIG. 43 is a perspective, assembly view of a brake of the articulation locking assembly of FIG. 40;

FIG. 44 is a perspective view of a collet of the articulation locking assembly of FIG. 40;

FIG. 45-49 are cross-sectional views of a portion of the outer tube of the adapter assembly of FIG. 20 illustrating the articulation locking assembly and the firing rod assembly of FIG. 40 during various stages of use;

FIG. 50 is a perspective view of a distal portion of the outer tube of the adapter assembly of FIG. 20 illustrating an articulation control mechanism;

FIG. 51 is an enlarged view of the area of detail indicated in FIG. 50;

FIG. 52 is a perspective, assembly view of the articulation control mechanism of FIG. 50;

FIG. 53 is a cut-away perspective view taken along line 53-53 in FIG. 50;

FIG. 54 is a cross-sectional view of the articulation control mechanism of FIG. 50 in a proximal position;

FIG. 55 is a top view of a distal portion of the articulation control mechanism of FIG. 50 in a distal position;

FIG. 56 is a cross-section view taken along line 56-56 in FIG. 55 illustrating the articulation control mechanism in a distal position;

FIGS. 57 and 58 are perspective views of the end effector assembly of FIG. 20;

FIG. 59 is a cross-sectional view of a distal portion of the outer tube and a proximal portion of the end effector assembly taken along line 59-59 of FIG. 58;

FIG. 60 is a perspective, assembly view of an I-beam of the adapter assembly in accordance with embodiments of the present disclosure;

FIGS. 61 and 62 are perspective views of the I-beam of FIG. 60 engaged with a distal end of a drive beam assembly in accordance with embodiments of the present disclosure;

FIG. 63 is a perspective, assembly view of a pivot assembly of the adapter assembly in accordance with embodiments of the present disclosure;

FIG. 64 is a cross-sectional view of a distal portion of the outer tube and a proximal portion of the end effector assembly taken along line 64-64 of FIG. 58;

FIG. 65 is a perspective view of a blowout plate of the adapter assembly in accordance with embodiments of the present disclosure;

FIG. 65a is a side view of an alternate embodiment of a blowout plate engaged with pivot portions of the adapter assembly in accordance with embodiments of the present disclosure;

FIG. 66 is a perspective view of the end effector of the adapter assembly of FIG. 20 oriented in an articulated position relative to the outer tube in accordance with embodiments of the present disclosure;

FIG. 67 is a cross-sectional view of a distal portion of the outer tube and a proximal portion of the end effector assembly taking along line 67-67 in FIG. 66;

FIG. 68 is a perspective, assembly view of the end effector assembly of FIG. 20 including a sled detection mechanism in accordance with embodiments of the present disclosure;

FIG. 69 is a perspective view of various components of the sled detection mechanism of FIG. 68, shown assembled within a proximal portion of the end effector assembly, and separated from the end effector assembly;

FIGS. 70-77 are perspective views of portions of the sled detection mechanism of FIG. 68 during various stages of use;

FIG. 78 is a perspective, assembly view of the end effector assembly of FIG. 20 including a knife detection mechanism in accordance with embodiments of the present disclosure;

FIG. 79 is a perspective, assembly view of portions of the end effector assembly of FIG. 78;

FIG. 80 is a perspective view of a lockout spring of the knife detection mechanism of FIG. 78;

FIG. 81 is a perspective view of a knife of the knife detection mechanism of FIG. 78;

FIG. 82 is a cross-sectional view of the end effector assembly of FIG. 78 in an open position;

FIG. 83 is an enlarged view of the area of detail indicated in FIG. 82;

FIGS. 84 and 85 are cross-sectional views of a proximal portion of the end effector assembly of FIG. 78 illustrating the knife detection mechanism of FIG. 78 during various stages of use;

FIG. 86 is a perspective view of the adapter assembly of FIG. 20 illustrating a cartridge separated from a cartridge channel, and illustrating portions of an electrical assembly within the adapter assembly;

FIG. 87 is an enlarged view of the area of detail indicated in FIG. 86;

FIG. 88 is a perspective view of portions of the electrical assembly of FIG. 86;

FIG. 89 is a perspective view of a proximal portion of the adapter assembly of FIG. 20, with portions omitted, illustrating portions of the electrical assembly of FIG. 86;

FIG. 90 is a perspective view of the portions of the electrical assembly of FIG. 89;

FIG. 91 is a perspective view of the proximal portion of the adapter assembly of FIG. 20, with portions omitted, illustrating portions of the electrical assembly of FIG. 86;

FIG. 92 is a perspective view of a distal portion of the outer tube and a proximal portion of the end effector assembly of the adapter assembly of FIG. 20, illustrating portions of the electrical assembly of FIG. 86;

FIG. 93 is a perspective view of portions of the electrical assembly of FIG. 92;

FIG. 94 is a cross-sectional view including portions of the electrical assembly of FIG. 86 within a distal portion of the outer tube and a proximal portion of the end effector assembly of the adapter assembly of FIG. 20, illustrating the end effector in a non-articulated position;

FIG. 95 is a cross-sectional view including portions of the electrical assembly of FIG. 86 within a distal portion of the outer tube and a proximal portion of the end effector assembly of the adapter assembly of FIG. 20, illustrating the end effector in an articulated position;

FIG. 96 is a perspective view of the end effector assembly of FIG. 20 and including a portion of the electrical assembly of FIG. 86;

FIG. 97 is a perspective view of the cartridge of FIG. 20 and including a portion of the electrical assembly of FIG. 86;

FIG. 98 is a perspective view of a proximal portion of the cartridge shown in FIG. 97;

FIG. 99 is a perspective view of the cartridge of FIG. 20 including a portion of an electrical assembly in accordance with embodiments of the present disclosure;

FIG. 100 is a perspective view of a proximal portion of the cartridge shown in FIG. 99;

FIG. 101 is a perspective view of the cartridge of FIG. 20 including a portion of an electrical assembly in accordance with embodiments of the present disclosure;

FIG. 102 is a perspective view of a proximal portion of the cartridge shown in FIG. 101;

FIG. 103 is a perspective, cut-away view of an end effector assembly including the proximal portion of the cartridge shown in FIGS. 101 and 102;

FIG. 104 is a perspective view of a proximal portion of a cartridge including a portion of an electrical assembly in accordance with embodiments of the present disclosure;

FIG. 105 is a perspective, cut-away view of an end effector assembly including the proximal portion of the cartridge shown in FIG. 104;

FIG. 106 is a perspective view of a proximal portion of a cartridge including a portion of an electrical assembly in accordance with embodiments of the present disclosure;

FIG. 107 is a perspective, cut-away view of an end effector assembly including the proximal portion of the cartridge shown in FIG. 106;

FIG. 108 is a perspective view of a distal portion of the adapter assembly of FIG. 20 including portions of an electrical assembly in accordance with embodiments of the present disclosure;

FIG. 109 is a perspective view of the portions of the electrical assembly of FIG. 108;

FIG. 110 is a perspective view including portions of the electrical assembly of FIG. 108 within a distal portion of the outer tube and a proximal portion of the end effector assembly of the adapter assembly of FIG. 20;

FIG. 111 is a perspective view of a distal portion of the adapter assembly of FIG. 20 including portions of an electrical assembly in accordance with embodiments of the present disclosure;

FIG. 112 is an enlarged view of the area indicated in FIG. 111;

FIG. 113 is an enlarged view of the area indicated in FIG. 111;

FIG. 114 is a perspective view of portions of an electrical assembly within the outer tube of the adapter assembly of FIG. 20 in accordance with embodiments of the present disclosure;

FIG. 115 is a perspective view of a portion of the adapter assembly of FIG. 20 illustrating a pivot link and a portion of an electrical assembly;

FIGS. 116 and 117 are various views of a pivot link and a portion of an electrical assembly engaged therewith in accordance with embodiments of the present disclosure;

FIG. 118 is a perspective view of a pivot link and a portion of an electrical assembly engaged therewith in accordance with embodiments of the present disclosure;

FIG. 119 is a perspective view of a portion of the adapter assembly of FIG. 20 illustrating a pivot link and a portion of an electrical assembly engaged therewith in accordance with embodiments of the present disclosure;

FIGS. 120 and 121 are various views of a pivot link and a portion of an electrical assembly engaged therewith in accordance with embodiments of the present disclosure;

FIG. 122 is a perspective view of a pivot link and a portion of an electrical assembly engaged therewith in accordance with embodiments of the present disclosure;

FIG. 123 is a perspective view of a proximal portion of the adapter assembly of FIG. 20, with portions omitted, and including portions of an electrical assembly in accordance with embodiments of the present disclosure;

FIG. 124 is a cross-sectional view of the proximal portion of the adapter assembly and the electrical assembly shown in FIG. 123;

FIG. 125 is a perspective view of the proximal portion of the adapter assembly and the electrical assembly shown in FIG. 123, with portions omitted;

FIG. 126 is a transverse cross-sectional view of the proximal portion of the adapter assembly and the electrical assembly shown in FIG. 123;

FIG. 127 is a perspective view of a proximal portion of the adapter assembly of FIG. 20, with portions omitted, and including portions of an electrical assembly in accordance with embodiments of the present disclosure;

FIG. 128 is a perspective view of a portion of the electrical assembly of FIG. 127;

FIG. 129 is a perspective view of a distal portion of the adapter assembly of FIG. 20 illustrating a strain gauge on the end effector assembly in accordance with embodiments of the present disclosure;

FIG. 130 is a perspective view of the proximal portion of the adapter assembly and the electrical assembly shown in FIG. 123, with portions omitted;

FIGS. 131-133 are cross-sectional views of the proximal portion of the adapter assembly and the electrical assembly shown in FIG. 123 during varying stages of rotation; and

FIG. 134 is a schematic illustration of a robotic surgical system configured for use in accordance with the disclosure.

### DETAILED DESCRIPTION

Embodiments of the presently disclosed surgical devices, and adapter assemblies for surgical devices and/or handle assemblies are described in detail with reference to the drawings, in which like reference numerals designate identical or corresponding elements in each of the several views. As used herein the term “distal” refers to that portion of the adapter assembly or surgical device, or component thereof, farther from the user, while the term “proximal” refers to that portion of the adapter assembly or surgical device, or component thereof, closer to the user. As used herein, the terms parallel and perpendicular are understood to include relative configurations that are substantially parallel or substantially perpendicular up to about +or −10 degrees from true parallel or true perpendicular, respectively.

In the following description, well-known functions or construction are not described in detail to avoid obscuring the present disclosure in unnecessary detail.

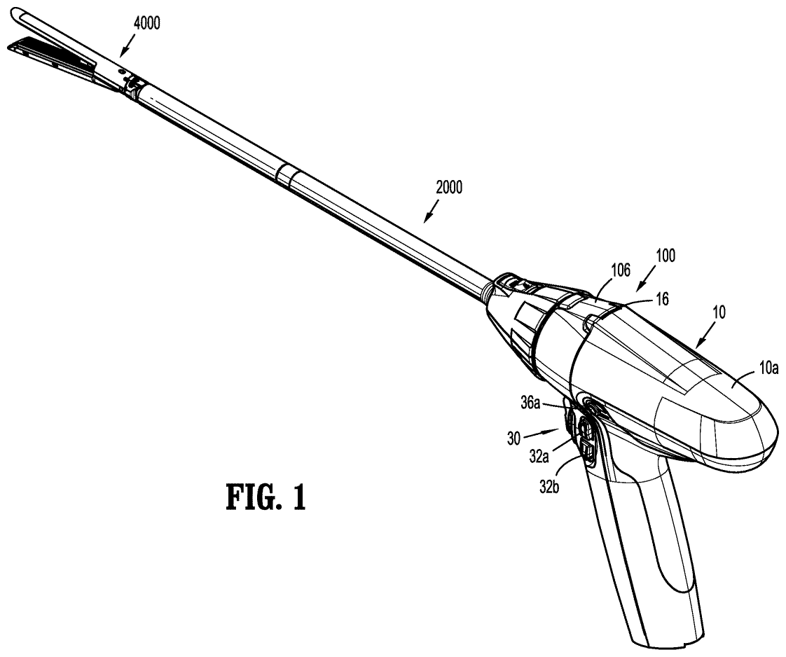

A surgical device, in accordance with an embodiment of the present disclosure, is generally designated as 100, and is in the form of a powered handheld electromechanical instrument. As illustrated in FIG. 1, the surgical device 100 is configured for selective connection with an adapter assembly 2000. Although described with respect to the adapter assembly 2000, different adapter assemblies having or configured for use with different end effector assemblies 4000 are also capable of being used with the surgical device 100.

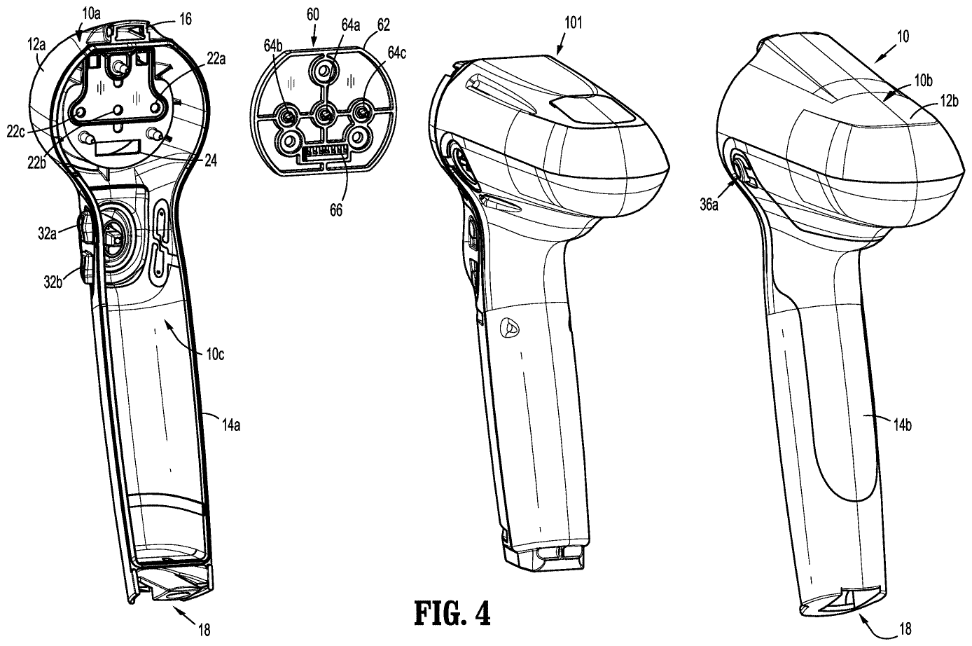

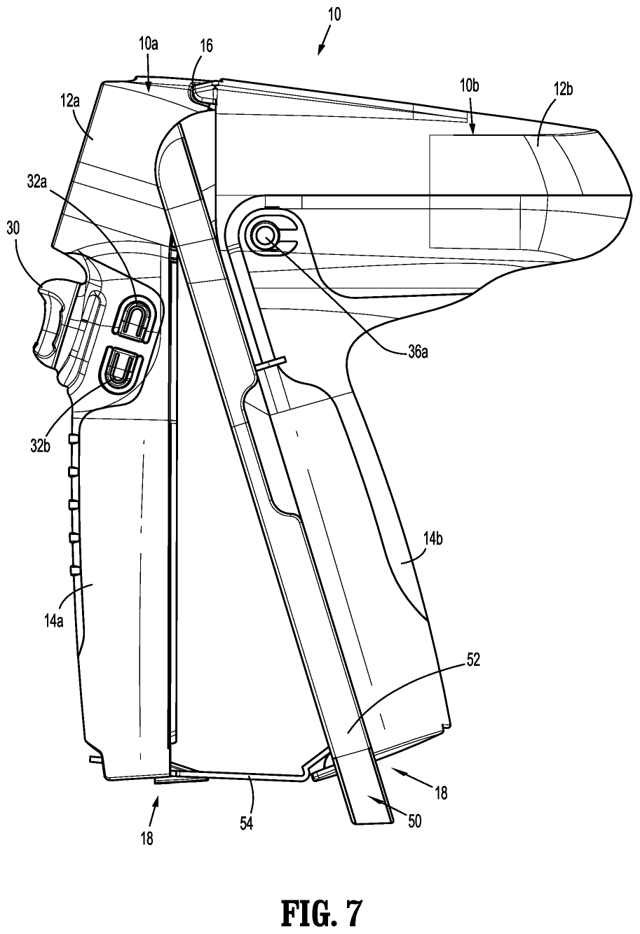

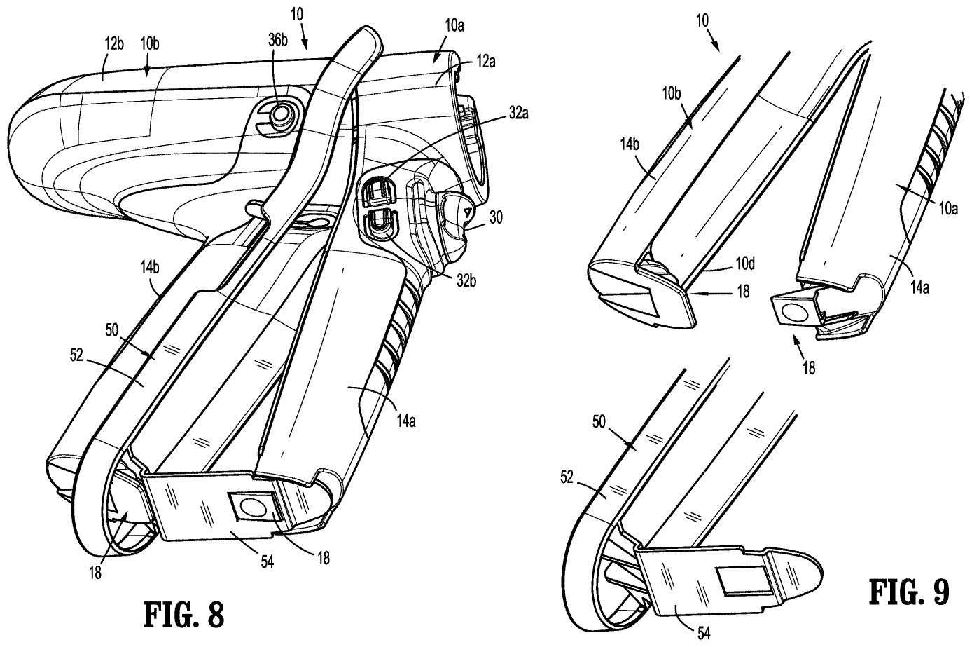

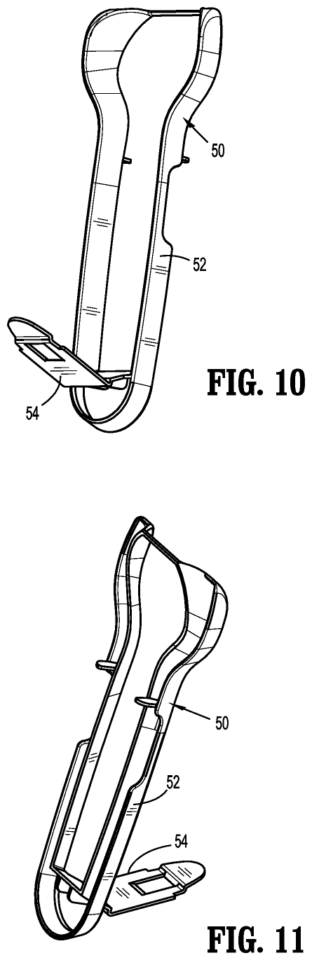

As illustrated in FIGS. 1-11, the surgical device 100 includes a power-pack 101, and an outer shell housing 10 configured to selectively receive and sealingly encase the power-pack 101 to establish a sterile barrier about the power-pack 101. The outer shell housing 10 includes a distal half-section 10a and a proximal half-section 10b pivotably connected to distal half-section 10a by a hinge 16 located along an upper edge of the distal half-section 10a and the proximal half-section 10b. When joined, the distal half-section 10a and the proximal half-section 10b define a shell cavity 10c therein in which the power-pack 101 is selectively situated.

The distal half-section 10a and the proximal half-section 10b are divided along a plane that traverses a longitudinal axis “X-X” of the adapter assembly 2000.

Each of the distal half-section 10a and the proximal half-section 10b includes a respective upper shell portion 12a, 12b, and a respective lower shell portion 14a, 14b. The lower shell portions 12a, 12b define a snap closure feature 18 for selectively securing the lower shell portions 12a, 12b to one another and for maintaining the outer shell housing 10 in a closed condition.

The distal half-section 10a of the outer shell housing 10 defines a connecting portion 20 configured to accept a corresponding drive coupling assembly 210 of the adapter assembly 2000. Specifically, the distal half-section 10a of the outer shell housing 10 has a recess 20 that receives a portion of the drive coupling assembly 210 of the adapter assembly 2000 when the adapter assembly 2000 is mated with the surgical device 100.

The connecting portion 20 of the distal half-section 10a defines a pair of axially extending guide rails 20a, 20b projecting radially inward from inner side surfaces thereof. The guide rails 20a, 20b assist in rotationally orienting the adapter assembly 2000 relative to the surgical device 100 when the adapter assembly 2000 is mated with the surgical device 100.

The connecting portion 20 of the distal half-section 10a defines three apertures 22a, 22b, 22c formed in a distally facing surface thereof and which are arranged in a common plane or line with one another. The connecting portion 20 of the distal half-section 10a also defines an elongate slot 24 also formed in the distally facing surface thereof.

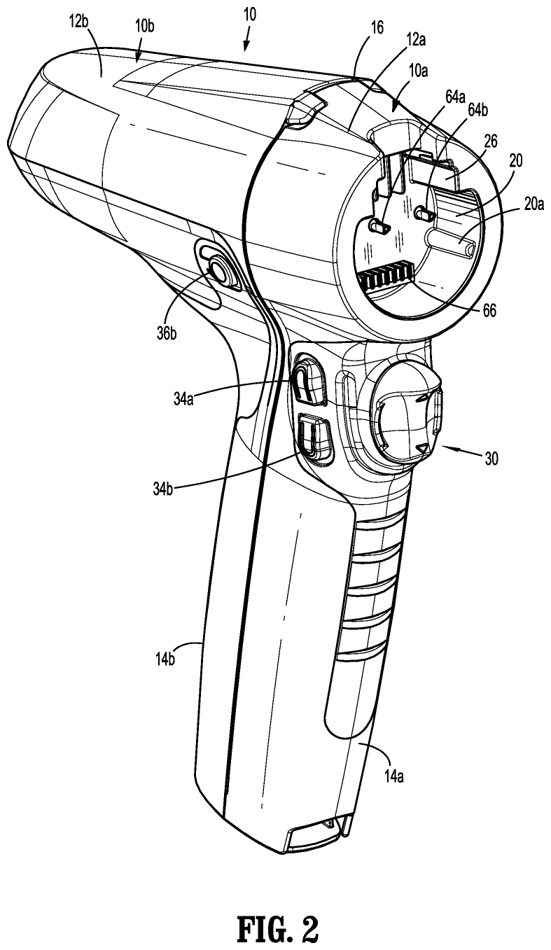

The connecting portion 20 of distal half-section 10a further defines a female connecting feature 26 (see FIG. 2) formed in a surface thereof. The female connecting feature 26 selectively engages with a male connecting feature of the adapter assembly 2000, as will be described in greater detail below.

The distal half-section 10a of the outer shell housing 10 supports a distal facing toggle control button 30. The toggle control button 30 is capable of being actuated in a left, right, up and down direction upon application of a corresponding force thereto or a depressive force thereto.

The distal half-section 10a of the outer shell housing 10 supports a right-side pair of control buttons 32a, 32b, and a left-side pair of control button 34a, 34b. The right-side control buttons 32a, 32b and the left-side control buttons 34a, 34b are capable of being actuated upon application of a corresponding force thereto or a depressive force thereto.

The proximal half-section 10b of the outer shell housing 10 supports a right-side control button 36a and a left-side control button 36b. The right-side control button 36a and the left-side control button 36b are capable of being actuated upon application of a corresponding force thereto or a depressive force thereto.

The distal half-section 10a and the proximal half-section 10b of the outer shell housing 10 are fabricated from a polycarbonate or similar polymer, and are transparent, translucent or opaque, and may be overmolded.

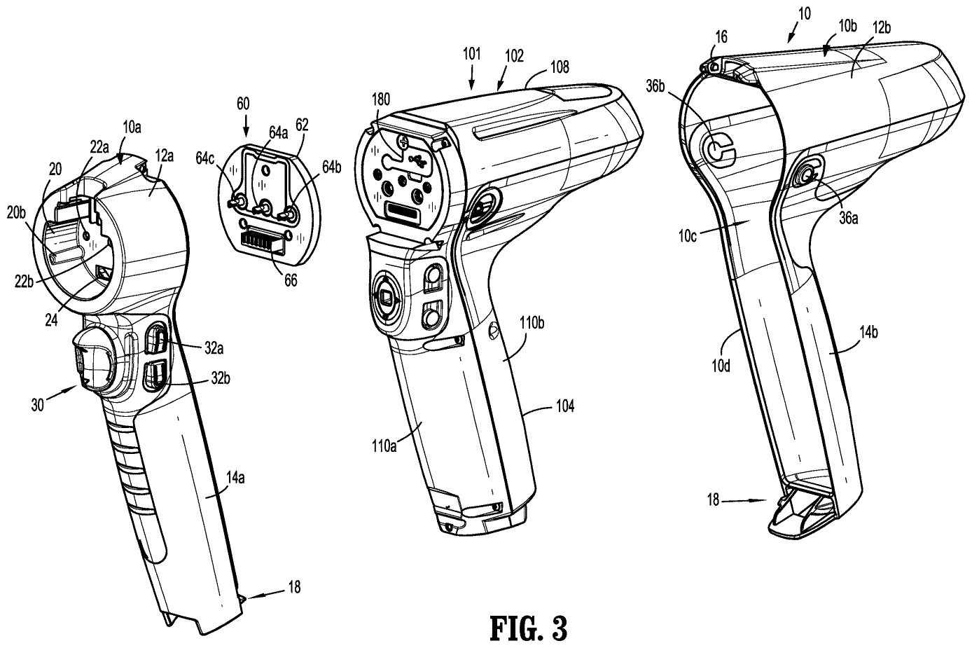

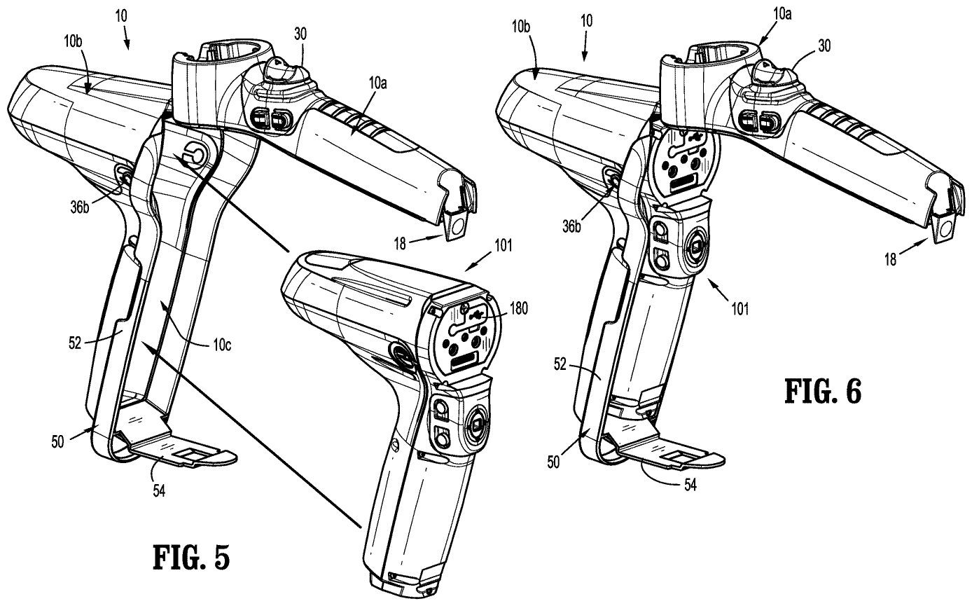

With reference to FIGS. 5-11, the surgical device 100 includes an insertion guide 50 that is configured and shaped to seat on and entirely surround a distal facing edge 10d (FIGS. 3 and 9) of the proximal half-section 10b. The insertion guide 50 includes a body portion 52 having a substantially U-shaped transverse cross-sectional profile, and a stand-off 54 extending from a bottom of the body portion 52. The stand-off 54 is configured to engage snap closure feature 18 of each of the lower shell portions 12a, 12b of respective distal and proximal half-sections 10a, 10b of the outer shell housing 10.

In use, when the body portion 52 of the insertion guide 50 is seated on the distal facing edge 10d of the proximal half-section 10b, the snap closure feature 18 of the lower shell portion 12a of the distal half-section 10a engages a first end of the stand-off 54, and the snap closure feature 18 of the lower shell portion 12b of the proximal half-section 10b engages a first end of the stand-off 54.

With reference to FIGS. 2-4, the outer shell housing 10 includes a sterile barrier plate assembly 60 selectively supported in the distal half-section 10a. Specifically, the sterile barrier plate assembly 60 is disposed proximally of the connecting portion 20 of the distal half-section 10a and within the shell cavity 10c of the outer shell housing 10. The plate assembly 60 includes a plate 62 rotatably supporting three coupling shafts 64a, 64b, 64c. Each coupling shaft 64a, 64b, 64c extends from opposed sides of the plate 62 and has a tri-lobe transverse cross-sectional profile. Each coupling shaft 64a, 64b, 64c extends through a respective aperture 22a, 22b, 22c of the connecting portion 20 of the distal half-section 10a when the sterile barrier plate assembly 60 is disposed within the shell cavity 10c of the outer shell housing 10.

The plate assembly 60 further includes an electrical pass-through connector 66 supported on the plate 62. The pass-through connector 66 extends from opposed sides of the plate 62. Each coupling shaft 64a, 64b, 64c extends through the aperture 24 of the connecting portion 20 of the distal half-section 10a when the sterile barrier plate assembly 60 is disposed within the shell cavity 10c of the outer shell housing 10. The pass-through connector 66 defines a plurality of contact paths each including an electrical conduit for extending an electrical connection across the plate 62.

When the plate assembly 60 is disposed within the shell cavity 10c of the outer shell housing 10, distal ends of the coupling shaft 64a, 64b, 64c and a distal end of the pass-through connector 66 are disposed or situated within the connecting portion 20 of the distal half-section 10a of the outer shell housing 10, and electrically and/or mechanically engage respective corresponding features of the adapter assembly 2000.

In operation, with a new and/or sterile outer shell housing 10 in an open configuration (i.e., the distal half-section 10a separated from the proximal half-section 10b, about the hinge 16), and with the insertion guide 50 in place against the distal edge of the proximal half-section 10b of the outer shell housing 10, the power-pack 101 is inserted into the shell cavity 10c of the outer shell housing 10. With the power-pack 101 inserted into the shell cavity 10c of the outer shell housing 10, the insertion guide 50 is removed from the proximal half-section 10b and the distal half-section 10a is pivoted, about the hinge 16, to a closed configuration for the outer shell housing 10. In the closed configuration, the snap closure feature 18 of the lower shell portion 12a of the distal half-section 10a engages the snap closure feature 18 of the lower shell portion 12b of the proximal half-section 10b.

In operation, following a surgical procedure, the snap closure feature 18 of the lower shell portion 12a of the distal half-section 10a is disengaged from the snap closure feature 18 of the lower shell portion 12b of the proximal half-section 10b, and the distal half-section 10a is pivoted, about the hinge 16, away from the proximal half-section 10b to open the outer shell housing 10. With the outer shell housing 10 open, the power-pack 101 is able to be removed from the shell cavity 10c of the outer shell housing 10 (specifically from the proximal half-section 10b of the outer shell housing 10), and the outer shell housing 10 can be discarded. The power-pack 101 can then be disinfected and cleaned.

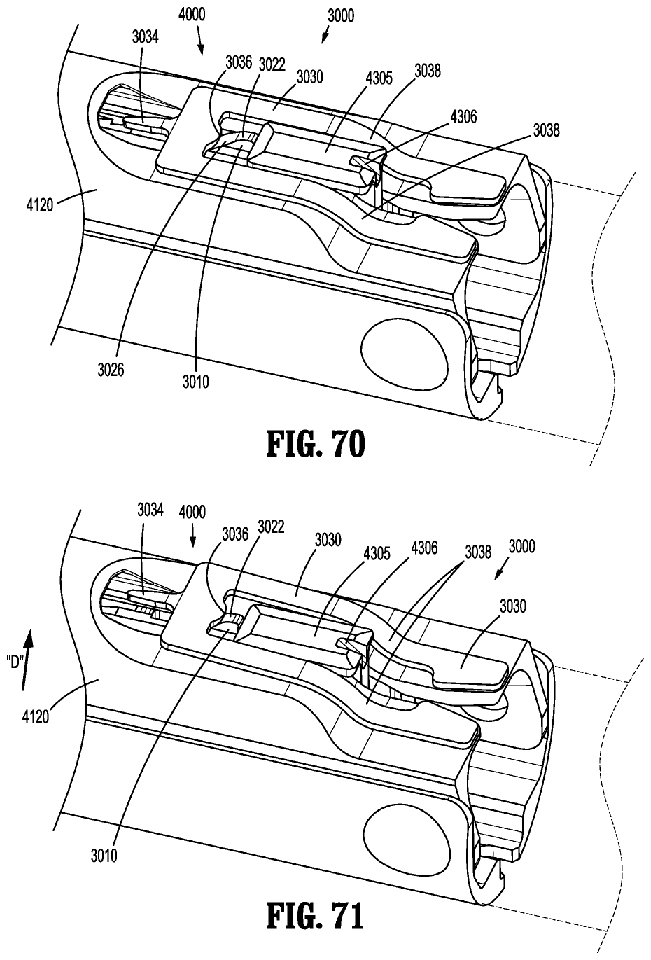

The outer shell housing 10, in addition to aseptically sealing the power-pack 101 when engaged thereabout, providing an operational interface for enabling operation of the surgical device 100 from the exterior of the outer shell housing 10, and including electrical and mechanical pass-through features for transmitting control and drive signals between the power-pack 101 and the other components of the surgical device 100, further includes a memory chip, e.g., a 1-wire chip, embedded therein. The memory chip includes a memory that stores a unique ID associated with the outer shell housing 10 and is capable of being updated to mark the outer shell housing 10 as “used.” The unique ID of the outer shell housing 10 allows for exclusive pairing of the outer shell housing 10 with a particular power-pack 101, while the ability to mark the outer shell housing 10 as “used” inhibits reuse of the outer shell housing 10, even with the same power-pack 101. Electrical contacts associated with the outer shell housing 10 form part of a 1-wire bus 171 (FIG. 70), or other suitable communication channel, that enables communication between the power-pack 101 and the 1-wire chip of outer shell housing 10. The 1-wire chip of the outer shell housing 10, for example, may be disposed on or within the plate assembly 60 thus enabling access thereto via one of the contact paths defined via the pass-through connector 66. Other locations and/or electrical couplings for enabling communication between the 1-wire chip of the outer shell housing 10 and power-pack 101 are also contemplated.

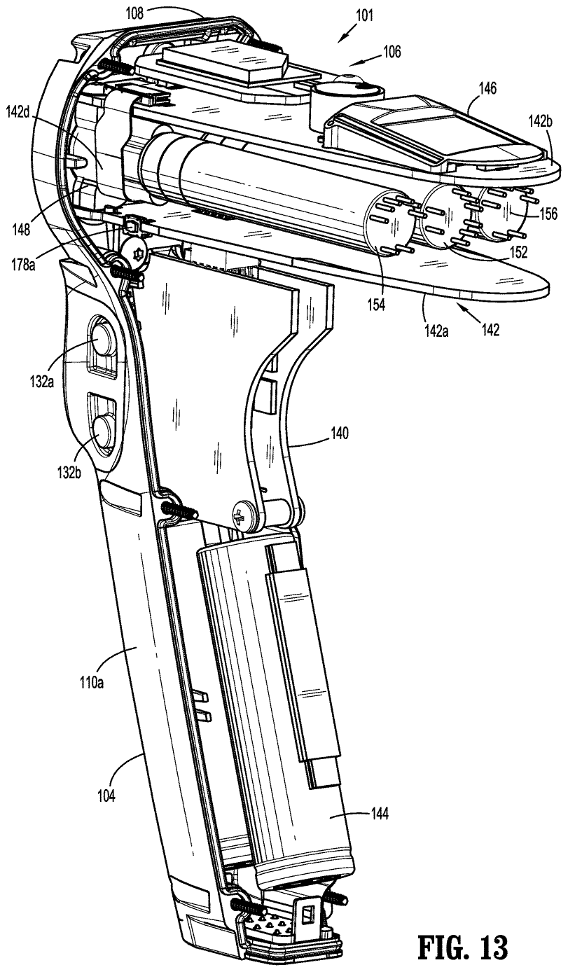

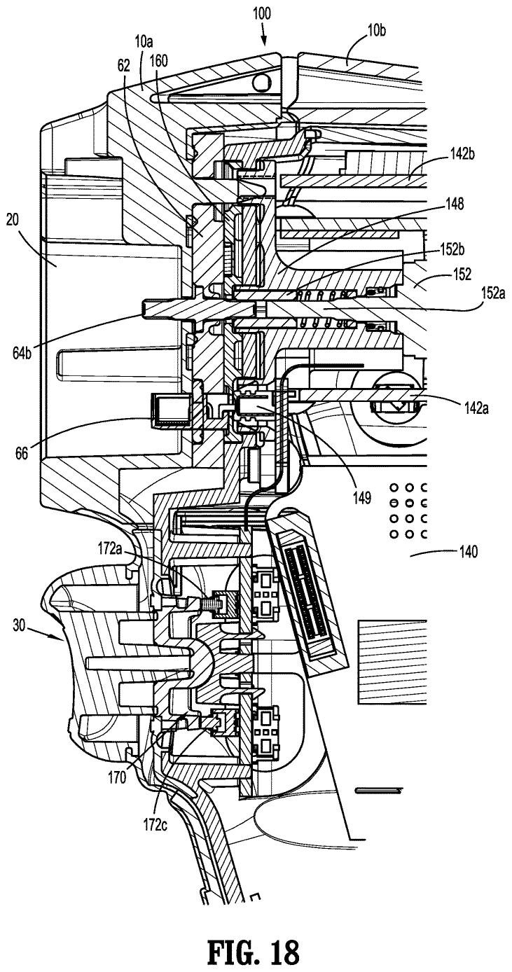

Referring to FIGS. 3-6 and FIGS. 12-19, the power-pack 101 includes an inner handle housing 110 having a lower housing portion 104 and an upper housing portion 108 extending from and/or supported on the lower housing portion 104. The lower housing portion 104 and the upper housing portion 108 are separated into a distal half-section 110a and a proximal half-section 110b connectable to one another by a plurality of fasteners. When joined, the distal half-section 110a and the proximal half-section 110b define the inner handle housing 110 having an inner housing cavity 110c therein in which a power-pack core assembly 106 is situated. The power-pack core assembly 106 is configured to control various operations of surgical device 100.

The distal half-section 110a of the inner handle housing 110 defines a distal opening 111a therein which is configured and adapted to support a control plate 160 of the power-pack core assembly 106. The control plate 160 of the power-pack 101 abuts against a rear surface of the plate 62 of the sterile barrier plate assembly 60 of the outer shell housing 10 when the power-pack 101 is disposed within the outer shell housing 10.

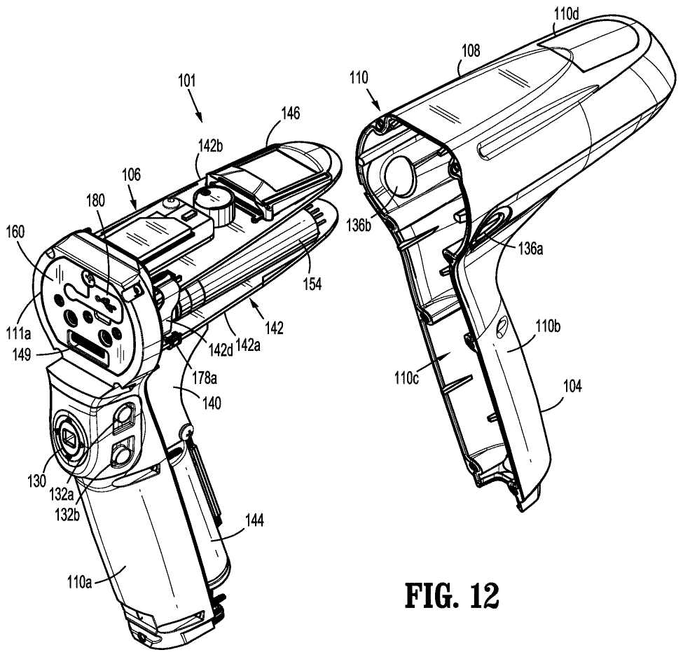

With reference to FIG. 12, the distal half-section 110a of the inner handle housing 110 supports a distal toggle control interface 130 that is in operative registration with the distal toggle control button 30 of the outer shell housing 10. In use, when the power-pack 101 is disposed within the outer shell housing 10, actuation of the toggle control button 30 exerts a force on the toggle control interface 130.

The distal half-section 110a of the inner handle housing 110 also supports a right-side pair of control interfaces 132a, 132b, and a left-side pair of control interfaces 134a, 134b. In use, when the power-pack 101 is disposed within the outer shell housing 10, actuation of one of the right-side pair of control buttons 32a, 32b or the left-side pair of control button 34a, 34b of the distal half-section 10a of the outer shell housing 10 exerts a force on a respective one of the right-side pair of control interfaces 132a, 132b or the left-side pair of control interfaces 134a, 134b of the distal half-section 110a of the inner handle housing 110.

In use, the right-side pair of control interfaces 132a, 132b or the left-side pair of control interfaces 134a, 134b of the distal half-section 110a of the inner handle housing 110 will be deactivated or fail to function unless the outer shell housing 10 has been validated.

The proximal half-section 110b of the inner handle housing 110 defines a right-side control aperture 136a and a left-side control aperture 136b. In use, when the power-pack 101 is disposed within the outer shell housing 10, actuation of one of the right-side control button 36a or the left-side control button 36b of the proximal half-section 10b of the outer shell housing 10 extends the right-side control button 36a or the left-side control button 36b into and across the right-side control aperture 136a or the left-side control aperture 136b of the proximal half-section 110b of the inner handle housing 110.

With reference to FIGS. 12-19, the inner handle housing 110 provides a housing in which the power-pack core assembly 106 is situated. The power-pack core assembly 106 includes a rechargeable battery 144 configured to supply power to any of the electrical components of the surgical device 100, a battery circuit board 140, and a controller circuit board 142. The controller circuit board 142 includes a motor controller circuit board 142a, a main controller circuit board 142b, and a first ribbon cable 142c interconnecting the motor controller circuit board 142a and the main controller circuit board 142b. The motor controller circuit board 142a is communicatively coupled with the battery circuit board 140 enabling communication therebetween and between the battery circuit board 140 and the main controller circuit board 142b.

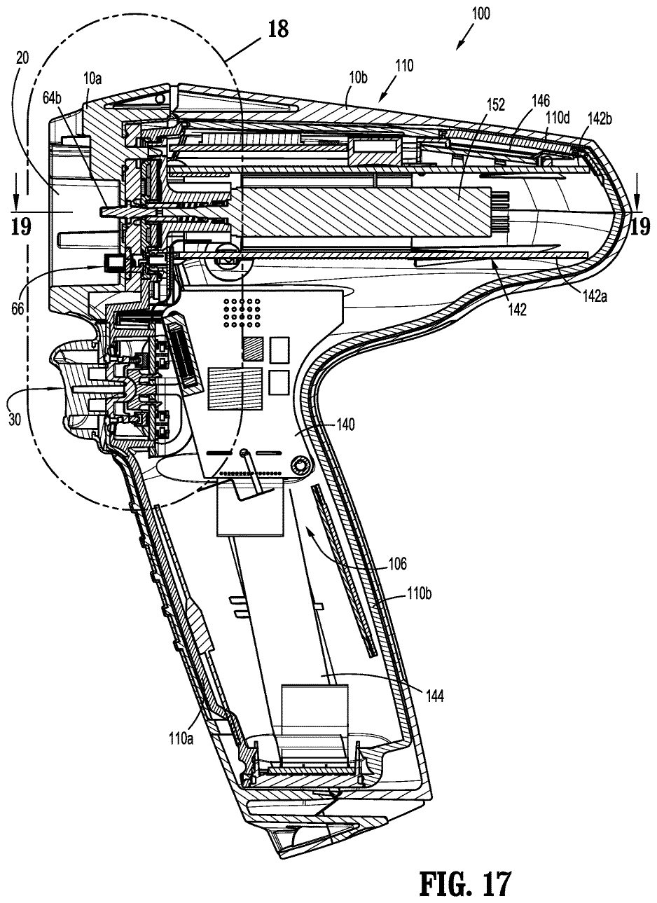

The power-pack core assembly 106 further includes a display screen 146 supported on the main controller circuit board 142b. The display screen 146 is visible through a transparent or translucent window 110d (see FIGS. 12 and 17) provided in the proximal half-section 110b of the inner handle housing 110. It is contemplated that at least a portion of the inner handle housing 110 may be fabricated from a transparent, translucent or opaque rigid plastic or the like. It is further contemplated that the outer shell housing 10 may either include a window formed therein (in visual registration with the display screen 146 and with the window 110d of the proximal half-section 110b of the inner handle housing 110, and/or the outer shell housing 10 may be fabricated from a transparent, translucent or opaque rigid plastic or the like.

The power-pack core assembly 106 further includes a first motor 152, a second motor 154, and a third motor 156 each electrically connected to the controller circuit board 142 and the battery 144. The motors 152, 154, 156 are disposed between the motor controller circuit board 142a and the main controller circuit board 142b. Each motor 152, 154, 156 includes a respective motor shaft 152a, 154a, 156a extending therefrom. Each motor shaft 152a, 154a, 156a has a tri-lobe transverse cross-sectional profile for transmitting rotative forces or torque. As an alternative to the motors 152, 154, 156, it is envisioned that more or fewer motors are provided or that one or more other drive components are utilized, e.g., a solenoid, and controlled by appropriate controllers. Manual drive components are also contemplated.

Each motor 152, 154, 156 is controlled by a respective motor controller “MC0,” MC1,” “MC2.” Motor controllers “MC0,” MC1,” “MC2” are disposed on the motor controller circuit board 142a. The motor controllers are disposed on motor controller circuit board 142a and are, for example, A3930/31K motor drivers from Allegro Microsystems, Inc. The A3930/31K motor drivers are designed to control a 3-phase brushless DC (BLDC) motor with N-channel external power MOSFETs, such as the motors 152, 154, 156. Each of the motor controllers is coupled to a main controller or master chip 157 disposed on the main controller circuit board 142b via the first ribbon cable 142c which connects the motor controller circuit board 142a with the main controller circuit board 142b. The main controller 157 communicates with motor controllers “MC0,” MC1,” “MC2” through a field-programmable gate array (FPGA) 162, which provides control logic signals (e.g., coast, brake, etc.). The control logic of motor controllers “MC0,” MC1,” “MC2” then outputs corresponding energization signals to respective motors 152, 154, 156 using fixed-frequency pulse width modulation (PWM). The main controller 157 is also coupled to memory 165, which is also disposed on the main controller circuit board 142b. The main controller 157 is, for example, an ARM Cortex M4 processor from Freescale Semiconductor, Inc, which includes 1024 kilobytes of internal flash memory.

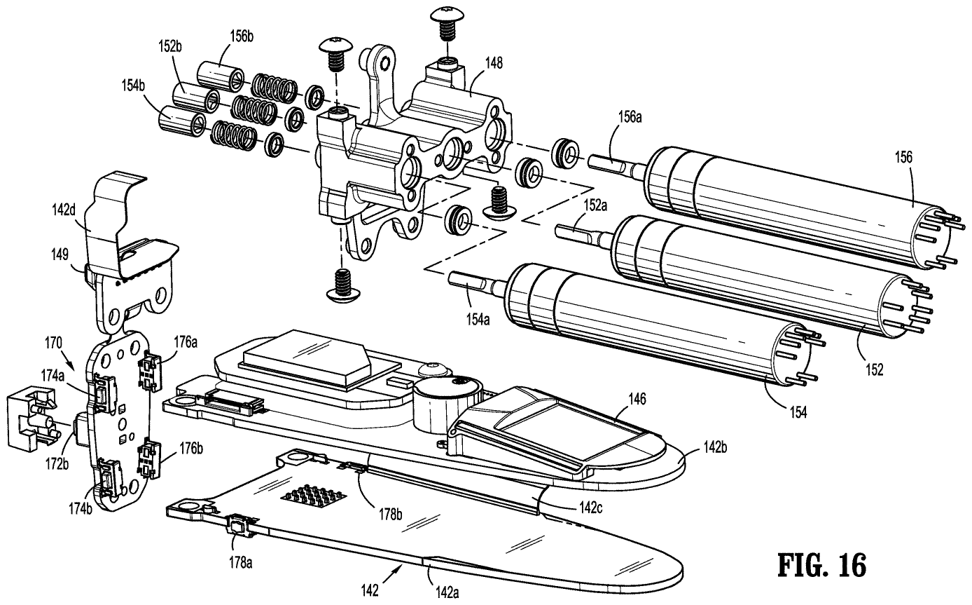

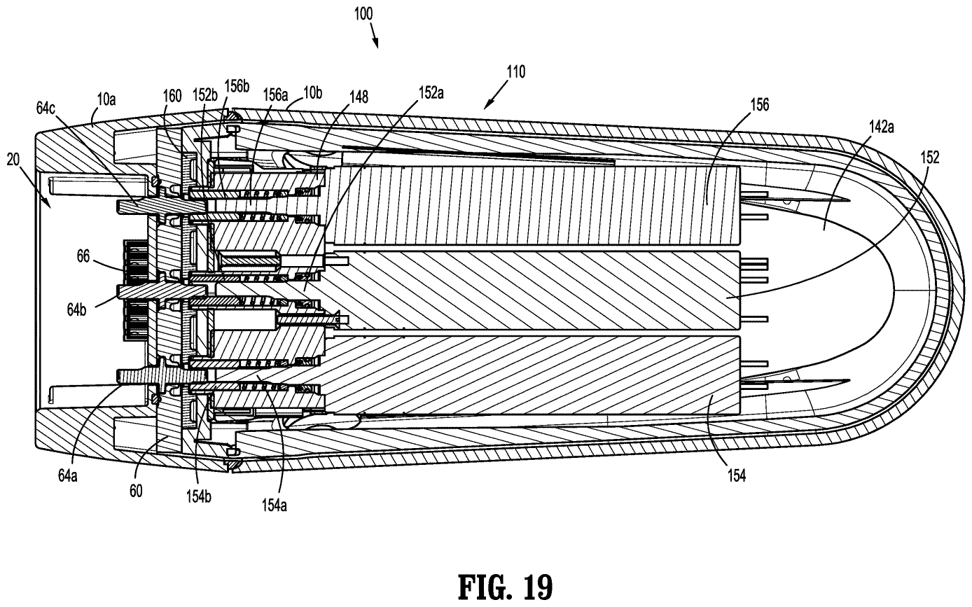

Each motor 152, 154, 156 is supported on a motor bracket 148 such that the motor shafts 152a, 154a, 156a are rotatably disposed within respective apertures of the motor bracket 148. As illustrated in FIGS. 16 and 19, the motor bracket 148 rotatably supports three rotatable drive connector sleeves 152b, 154b, 156b that are keyed to respective motor shafts 152a, 154a, 156a of motors 152, 154, 156. The drive connector sleeves 152b, 154b, 156b non-rotatably receive proximal ends of respective coupling shafts 64a, 64b, 64c of the plate assembly 60 of the outer shell housing 10, when the power-pack 101 is disposed within the outer shell housing 10. The drive connector sleeves 152b, 154b, 156b are each spring biased away from respective motors 152, 154, 156.

Rotation of the motor shafts 152a, 154a, 156a by respective motors 152, 154, 156 functions to drive shafts and/or gear components of the adapter assembly 2000 in order to perform the various operations of the surgical device 100. In particular, the motors 152, 154, 156 of the power-pack core assembly 106 are configured to drive shafts and/or gear components of the adapter assembly 2000 in order to selectively move an end effector assembly 4000, or portions thereof, to rotate the end effector assembly 4000 about the longitudinal axis “X-X,” to move a cartridge assembly 4120 relative to an anvil assembly 4140 of the end effector assembly 4000, and/or to fire fasteners from within the cartridge assembly 4120 of the end effector assembly 4000, for example.

The motor bracket 148 also supports an electrical adapter interface receptacle 149. The electrical receptacle 149 is in electrical connection with the main controller circuit board 142b by a second ribbon cable 142d. The electrical receptacle 149 defines a plurality of electrical slots for receiving respective electrical contacts or blades extending from the pass-through connector 66 of the plate assembly 60 of the outer shell housing 10.

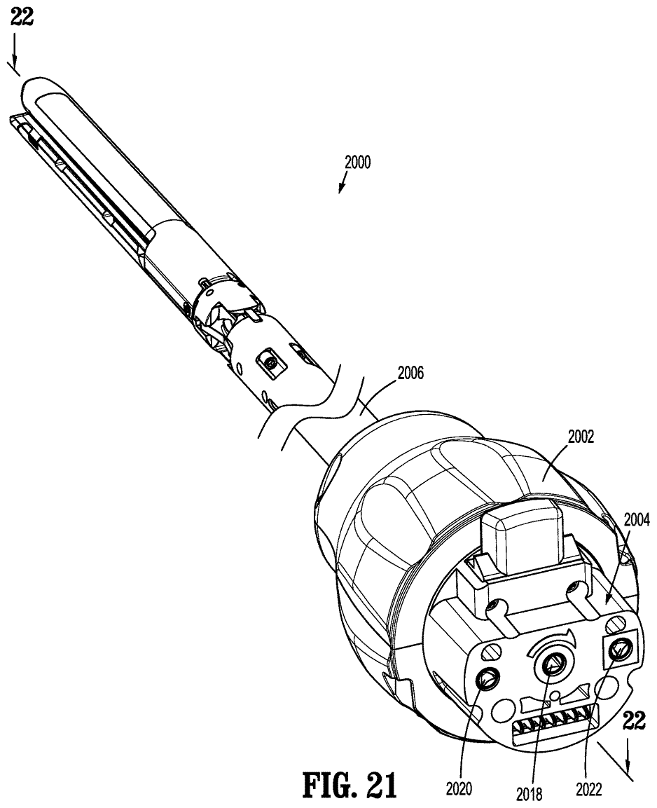

In use, when the adapter assembly 2000 is mated with the surgical device 100, each of the coupling shafts 64a, 64b, 64c of the plate assembly 60 of the outer shell housing 10 of the surgical device 100 couples with a corresponding rotatable connector sleeve 2018, 2020, 2022 of the adapter assembly 2000 (see FIG. 21). In this regard, the interface between the first coupling shaft 64a and the first connector sleeve 218, the interface between the second coupling shaft 64b and the second connector sleeve 220, and the interface between the third coupling shaft 64c and the third connector sleeve 222 are keyed such that rotation of each of the coupling shafts 64a, 64b, 64c of the surgical device 100 causes a corresponding rotation of the respective connector sleeve 218, 220, 222 of the adapter assembly 2000.

The mating of the coupling shafts 64a, 64b, 64c of the surgical device 100 with connector sleeves 218, 220, 222 of the adapter assembly 2000 allows rotational forces to be independently transmitted via each of the three respective connector interfaces. The coupling shafts 64a, 64b, 64c of the surgical device 100 are configured to be independently rotated by respective motors 152, 154, 156.

Since each of the coupling shafts 64a, 64b, 64c of the surgical device 100 has a keyed and/or substantially non-rotatable interface with respective connector sleeve 218, 220, 222 of the adapter assembly 2000, when the adapter assembly 2000 is coupled to the surgical device 100, rotational force(s) are selectively transferred from motors 152, 154, 156 of the surgical device 100 to the adapter assembly 2000. As will be discussed in greater detail below, the selective rotation of the coupling shaft(s) 64a, 64b, 64c of the surgical device 100 allows the surgical device 100 to selectively actuate different functions of the end effector assembly 4000.

With reference to FIGS. 12-19, the power-pack core assembly 106 further includes a switch assembly 170 supported within the distal half-section 110a of the inner handle housing 110, at a location beneath and in registration with the toggle control interface 130, the right-side pair of control interfaces 132a, 132b, and the left-side pair of control interfaces 134a, 134b. The switch assembly 170 includes a first set of four push-button switches 172a-172d arranged around a stem 30a of the toggle control button 30 of the outer shell housing 10 when the power-pack 101 is disposed within the outer shell housing 10. The switch assembly 170 also includes a second pair of push-button switches 174a, 174b disposed beneath the right-side pair of control interfaces 132a, 132b of the distal half-section 110a of the inner handle housing 110 when the power-pack 101 is disposed within the outer shell housing 10. The switch assembly 170 further includes a third pair of push-button switches 176a, 176b disposed beneath the left-side pair of control interfaces 134a, 134b of the distal half-section 110a of the inner handle housing 110 when power-pack 101 is disposed within the outer shell housing 10.

The power-pack core assembly 106 includes a single right-side push-button switch 178a disposed beneath the right-side control aperture 136a of the proximal half-section 110b of the inner handle housing 110, and a single left-side push-button switch 178b disposed beneath the left-side control aperture 136b of the proximal half-section 110b of the inner handle housing 110. The push-button switches 178a, 178b are supported on the controller circuit board 142, and are disposed beneath the right-side control button 36a and the left-side control button 36b of the proximal half-section 10b of the outer shell housing 10 when the power-pack 101 is disposed within the outer shell housing 10. Actuation of right-or left-side control button 36a, 36b actuates the respective right or left safety switches or keys 178a, 178b to permit entry of the power-pack core assembly 106 into the firing state. Entry into the firing state instructs the surgical device 100 that the end effector assembly 4000 is ready to expel fasteners therefrom, for instance.

The actuation of the push button switch 172c, corresponding to a downward actuation of the toggle control button 30, causes the controller circuit board 142 to provide appropriate signals to the motor 152 to approximate the cartridge assembly 4120 of the end effector assembly 4000 and/or to fire fasteners from within the cartridge assembly 4120 of the end effector assembly 4000.

The actuation of the push button switch 172a, corresponding to an upward actuation of the toggle control button 30, causes the controller circuit board 142 to provide appropriate signals to the motor 152 to retract an actuation sled 3010 and open the cartridge assembly 4120 of the end effector assembly 4000.

The actuation of the push button 172d, corresponding to an actuation of the toggle control button 30 to the right, causes the controller circuit board 142 to provide appropriate signals to the motor 152 to articulate the end effector assembly 4000 in a first direction. Similarly, actuation of the push button 172b, corresponding to an actuation of the toggle control button 30 to the left, causes the controller circuit board 142 to provide appropriate signals to the motor 152 to articulate the end effector assembly 4000 in a second, opposite, direction.

The actuation of the switches 174a, 174b (e.g., by right-hand thumb of user) or the switches 176a, 176b (e.g., by left-hand thumb of user), corresponding to respective actuation of the right-side pair of control buttons 32a, 32b or the left-side pair of control button 34a, 34b, causes the controller circuit board 142 to provide appropriate signals to the motor 154 to rotate the end effector assembly 4000 relative to an outer tube 2006 of an elongated shaft of the adapter assembly 2000. Specifically, actuation of the control button 32a or 34a causes the end effector assembly 4000 to rotate relative to the outer tube 2006 in a first direction, while actuation of control button 32b or 34b causes the end effector assembly 4000 to rotate relative to the outer tube 2006 in a second, opposite, direction.

In use, the cartridge assembly 4120 of the end effector assembly 4000 is actuated between opened and closed or approximated conditions as needed and/or desired. In order to fire the end effector assembly 4000, to expel fasteners therefrom, when the cartridge assembly 4120 of the end effector assembly 4000 is in a closed condition, the safety switch 178a or 178b is depressed thereby instructing the surgical device 100 that the end effector assembly 4000 is ready to expel fasteners therefrom.

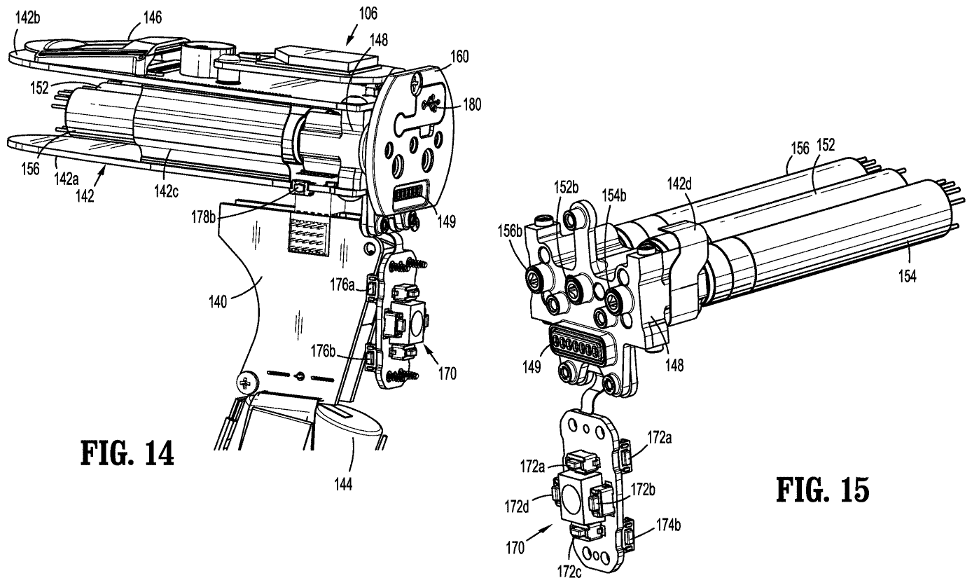

With reference to FIGS. 12 and 14, the power-pack core assembly 106 of the surgical device 100 includes a USB connector 180 supported on the main controller circuit board 142b of the controller circuit board 142. The USB connector 180 is accessible through the control plate 160 of the power-pack core assembly 106. When the power-pack 101 is disposed within the outer shell housing 10, the USB connector 180 is covered by the plate 62 of the sterile barrier plate assembly 60 of the outer shell housing 10.

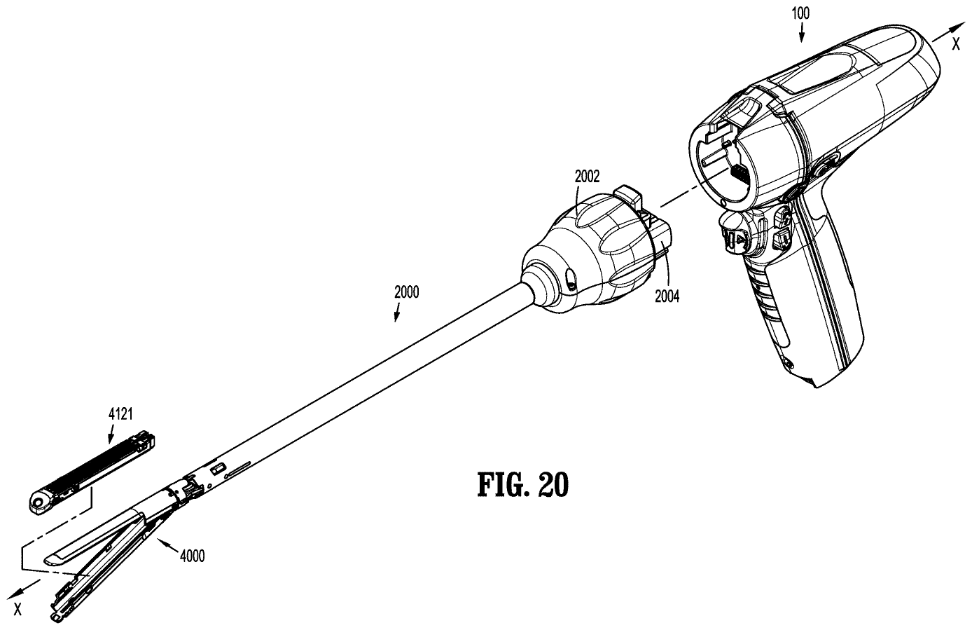

As illustrated in FIGS. 1 and 20, the surgical device 100 is configured for selective connection with one or more different types of adapters, e.g., the adapter assembly 2000. The adapter assembly 2000 is configured to convert a rotation of the drive connector sleeves 152b, 154b, 156b of the surgical device 100 into axial and/or rotational movement of portions of the adapter assembly 2000 and/or the end effector assembly 4000, as will be discussed in greater detail below.

Turning now to FIGS. 20-22, the adapter assembly 2000 includes a knob housing 2002 and the outer tube 2006 extending from a distal end of the knob housing 2002. The knob housing 2002 and the outer tube 2006 are configured and dimensioned to house the components of the adapter assembly 2000. The outer tube 2006 is dimensioned for insertion through a typical trocar port, cannula or the like. The knob housing 2002 is dimensioned to be too large to enter a typical trocar port, cannula of the like. The knob housing 2002 is configured and adapted to connect to the connecting portion 108 of the handle housing 102 of the surgical device 100.

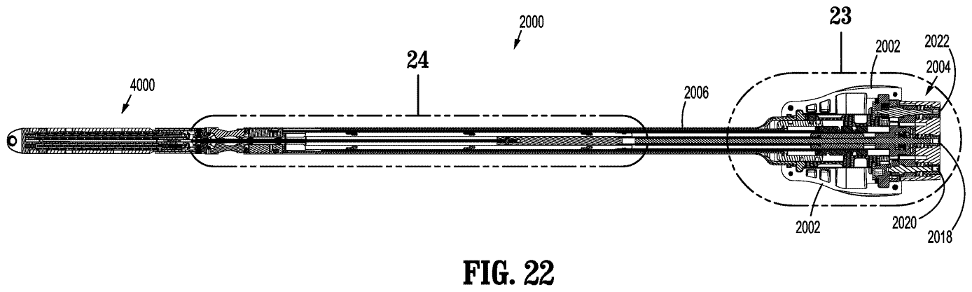

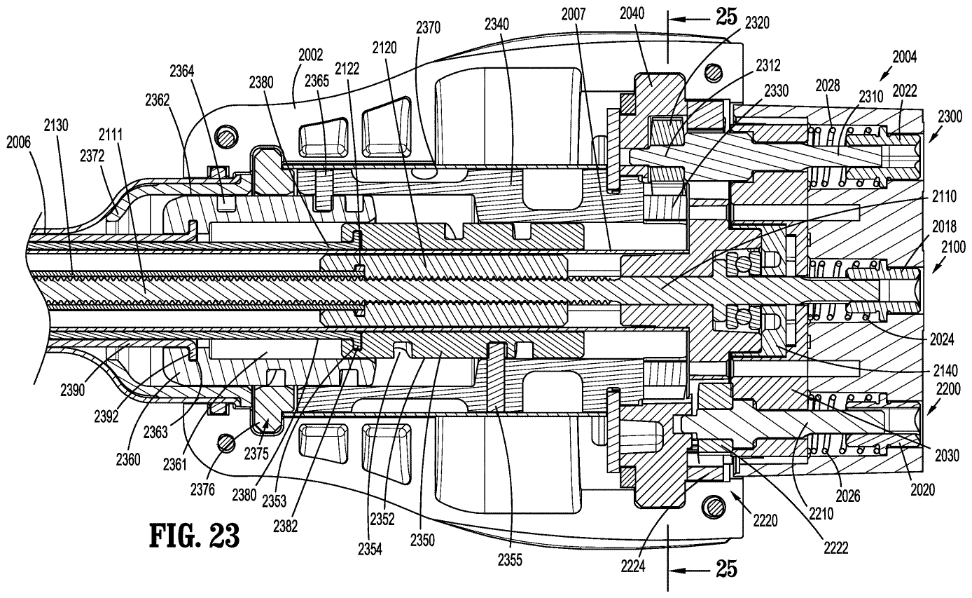

As illustrated in FIGS. 21 and 22, the adapter assembly 2000 includes a proximal housing assembly 2004 supporting a first connector sleeve 2018, a second connector sleeve 2020, and a third connector sleeve 2022 at least partially therein. Each connector sleeve 2018, 2020, 2022 functions as a rotation receiving member to receive rotational forces from the respective coupling shafts 64a, 64b and 64c of the surgical device 100, as described in greater detail below. Additionally, each of the connector sleeves 2018, 2020, 2022 is further configured to mate with a proximal end of the respective first, second and third proximal drive shafts 2110, 2210, 2310 (FIG. 23) of the adapter assembly 2000.

With reference to FIG. 23, a first biasing member 2024 is disposed distally of a portion of the first connector sleeve 2018, a second biasing member 2026 is disposed distally of a portion of the second connector sleeve 2020, and a third biasing member 2028 is disposed distally of a portion of the third connector sleeve 2022. Each biasing member 2024, 2026 and 2028 is disposed about respective first, second and third rotatable proximal drive shafts 2110, 2210 and 2310. The biasing members 2024, 2026 and 2028 act on respective connector sleeves 2018, 2020 and 2022 to help maintain the connector sleeves 2018, 2020 and 2022 engaged with the distal end of the respective coupling shafts 64a, 64c and 64b of the surgical device 100 when the adapter assembly 2000 is connected to the surgical device 100.

In particular, the first, second and third biasing members 2024, 2026 and 2028 function to bias the respective connector sleeves 2018, 2020 and 2022 in a proximal direction. In this manner, during connection of the adapter assembly 2000 to the surgical device 100, if the first, second and/or third connector sleeves 2018, 2020 and/or 2022 is/are poorly mated with the respective coupling shafts 64a, 64c and 64b of the surgical device 100, the first, second and/or third biasing member(s) 2024, 2026 and/or 2028 are compressed. Thus, when the surgical device 100 is operated, the coupling shafts 64a, 64c and 64b of the surgical device 100 will rotate, and the first, second and/or third biasing member(s) 2024, 2026 and/or 2028 will cause the respective first, second and/or third connector sleeve(s) 218, 2020 and/or 2022 to slide back proximally, effectively connecting the coupling shafts 64a, 64c and 64b of the surgical device 100 to the respective first, second and third proximal drive shafts 2110, 2210 and 2310 of the drive coupling assembly 2010.

With continued reference to FIG. 23, a plate bushing 2030 of the proximal housing assembly 2004 is shown. The plate bushing 2030 defines three apertures that are aligned with and rotatably receive the first, second and third proximal drive shafts 2110, 2210, 2310 therein. The plate bushing 2030 provides a surface against which the first, second and third biasing members 2024, 2026 and 2028 come into contact or rest against.

The adapter assembly 2000 includes a plurality of force/rotation transmitting/converting assemblies, each disposed within the proximal housing assembly 2004 and the outer tube 2006. Each force/rotation transmitting/converting assembly is configured and adapted to transmit/convert a speed/force of rotation (e.g., increase or decrease) of one of the first, second and third rotatable coupling shafts 64a, 64c and 64b of the surgical device 100 before transmission of such rotational speed/force to the end effector assembly 4000.

Specifically, as illustrated in FIG. 23, the adapter assembly 2000 includes an actuation assembly or a first force/rotation transmitting/converting assembly 2100, a rotation assembly or a second force/rotation transmitting/converting assembly 2200, and an articulation assembly or a third force/rotation transmitting/converting assembly 2300 disposed within the proximal housing assembly 2004 and the outer tube 2006. The first force/rotation transmitting/converting assembly 2100 is configured and adapted to transmit or convert a rotation of the first coupling shaft 64a of the surgical device 100 into axial translation of a firing rod assembly 2130 of the adapter assembly 2000 to effectuate closing, opening and/or firing of the end effector assembly 4000. The second force/rotation transmitting/converting assembly 2200 is configured and adapted to transmit or convert a rotation of the second coupling shaft 64b of the surgical device 100 into rotation of an annular gear 2224 of a ring gear assembly 2220 of the adapter assembly 2000, to effectuate rotation of portions of the adapter assembly 2000 relative to the outer shell housing 10. The third force/rotation transmitting/converting assembly 2300 is configured and adapted to transmit or convert a rotation of the third coupling shaft 64c of the surgical device 100 into axial translation of a first articulation tube 2380 and a second articulation tube 2390 of the adapter assembly 2000, to effectuate articulation of the end effector assembly 4000.

Additionally, the adapter assembly 2000 includes a core housing 2040 disposed within the outer knob housing 2002. The core housing 2040 defines a plurality of openings configured to allow portions of the first, second and third force/rotation transmitting/converting assemblies 2100, 2200, 2300 to pass through and/or engage.

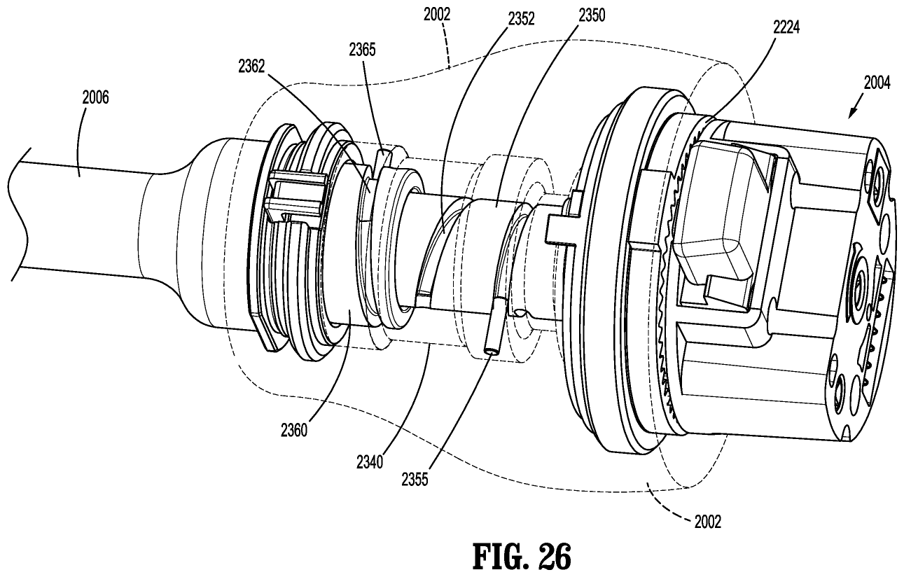

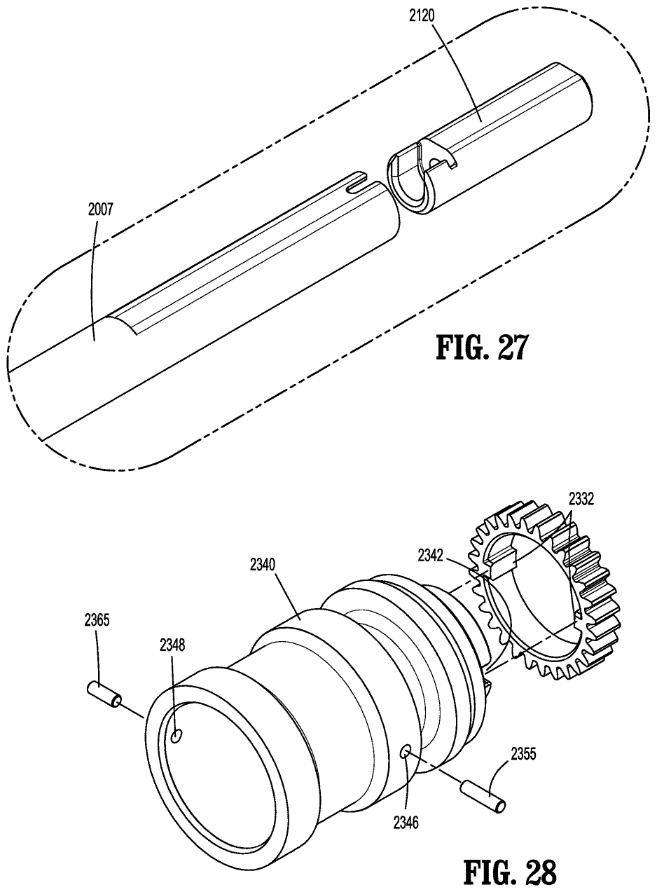

Referring now to FIGS. 23-30, details of the first force/rotation transmitting/converting assembly 2100, the second force/rotation transmitting/converting assembly 2200, and the third force/rotation transmitting/converting assembly 2300 are shown.

The first force/rotation transmitting/converting assembly 2100 includes the first proximal drive shaft 2110, which, as described above, is rotatably supported within the proximal housing assembly 2004. The first proximal drive shaft 2110 includes a non-circular shaped proximal end portion configured for connection with the first connector 2018 which is engageable with the first coupling shaft 64a of the surgical device 100. The first proximal drive shaft 2110 includes a distal portion 2111 having a threaded outer profile or surface.

The first force/rotation transmitting/converting assembly 2100 further includes a drive coupling nut 2120 rotatably coupled to threaded distal portion 2111 of the first proximal drive shaft 2110, and which is slidably disposed within a core tube 2007 relative to the first proximal drive shaft 2110. The drive coupling nut 2120 is slidably keyed within the core tube 2007 such that the drive coupling nut 2120 is prevented from rotation relative to the core housing 2040 as the first proximal drive shaft 2110 is rotated relative to the knob housing 2002. In this manner, as the first proximal drive shaft 2110 is rotated relative to the core housing 2040, the drive coupling nut 2120 is translated along the threaded distal portion 2111 of the first proximal drive shaft 2110 and, in turn, through and/or along a proximal portion of the outer tube 2006. More particularly, as the first proximal drive shaft 2110 is rotated in a first direction (e.g., clockwise), the drive coupling nut 2120 is translated distally along the threaded distal portion 2111 of the first proximal drive shaft 2110, and as the first proximal drive shaft 2110 is rotated in a second direction (e.g., counter-clockwise), the drive coupling nut 2120 is translated proximally along the threaded distal portion 2111 of the first proximal drive shaft 2110.

The first force/rotation transmitting/converting assembly 2100 further includes the firing rod assembly 2130 that is mechanically engaged with the drive coupling nut 2120, such that longitudinal movement of the drive coupling nut 2120 results in a corresponding amount and direction of longitudinal movement of the firing rod assembly 2130. The drive coupling nut 2120 and/or the firing rod assembly 2130 function as a force transmitting member to components of the end effector assembly 4000, as described in greater detail below. As shown, the drive coupling nut 2120 defines a distal recess 2122 through which a portion of the firing rod assembly 2130 extends into. The drive coupling nut 2120 is fixed (i.e., non-rotatably) to the core tube 2007. A proximal portion of the firing rod assembly 2130 is rotatably fixed to the distal recess 2122 of the drive coupling nut 2120.

Additionally, a sensor 2140 is provided adjacent the plate bushing 2030 of the proximal housing assembly 2004. As shown in FIG. 23, the first proximal drive shaft 2110 extends through an aperture defined by the sensor 2140. In embodiments, the sensor 2140 includes a strain gauge, a pressure sensor, etc. When a strain gauge is utilized, the strain gauge provides a closed-loop feedback to a firing/clamping load exhibited by first proximal drive shaft 2110, based upon which the power-pack core assembly 106 sets the speed current limit on the appropriate motor 152, 154, 156, for instance.

In operation, rotation of the first coupling shaft 64a of the surgical device 100, results in rotation of the first connector sleeve 2018, which results in rotation of the first proximal drive shaft 2110 relative to the core housing 2040. In response to the rotation of the first proximal drive shaft 2110, the drive coupling nut 2120 moves longitudinally along the threaded distal portion 2111 of the first proximal drive shaft 2110 relative to the first proximal drive shaft 2110. Further, longitudinally movement of the drive coupling nut 2120 causes the firing rod assembly 2130 to move longitudinally relative to the outer tube 2006. The longitudinally translation of the firing rod assembly 2130 causes concomitant distal translation of a drive assembly 4200 (or portions thereof) of the end effector assembly 4000 to effectuate an approximation of the cartridge assembly 4120 and the anvil assembly 4140, and/or ejection of fasteners from the cartridge assembly 4120, cutting tissue between the cartridge assembly 4120 and the anvil assembly 4140, and/or retraction of the drive assembly 4200 (or portions thereof), for instance.

With continued reference to FIGS. 23-30, the second force/rotation transmitting/converting assembly 2200 includes the second proximal drive shaft 2210, which, as described above, is rotatably supported within the proximal housing assembly 2004. The second proximal drive shaft 2210 includes a non-circular shaped proximal end portion configured for connection with the second connector 2020 which is engageable with the second coupling shaft 64b of the surgical device 100. The second proximal drive shaft 2210 is engaged with a ring gear assembly 2220 for rotation of portions of the adapter assembly 2000 and the end effector assembly 4000 relative to the core housing 2040.

With particular reference to FIGS. 23, 26 and 29, the ring gear assembly 2220 includes an inner, pinion gear 2222 and the outer, annular gear 2224. The pinion gear 2222 defines an outer array of gear teeth and is engaged with or unitarily formed with the second proximal drive shaft 2210. The annular gear 2224 defines an inner array of gear teeth and is non-rotatably coupled to the knob housing 2002. The gear teeth of the pinion gear 2222 mesh with the gear teeth of the annular gear 2224. The non-rotatable engagement between the annular gear 2224 and the knob housing 2002 enables rotation of the annular gear 2224 to cause a corresponding rotation of the knob housing 2002 relative to the core housing 2040, and vice versa.

Further, the knob housing 2002 is non-rotatably coupled to the outer tube 2006 such that rotation of the knob housing 2002 results in a corresponding rotation of the outer tube 2006. Additionally, the end effector assembly 4000 is non-rotatably coupled to the outer tube 2006, such that rotation of the outer tube 2006 results in a corresponding rotation of the end effector assembly 4000.

In use, rotation of the second coupling shaft 64b of the surgical device 100, results in rotation of the second connector sleeve 2020, which results in rotation of the second proximal drive shaft 2210 relative to the proximal housing assembly 2004. Rotation of the second proximal drive shaft 2210 results in rotation of the knob housing 2002, the outer tube 2006, and the end effector assembly 4000 relative to the proximal housing assembly 2004. Additionally, the knob housing 2002 is rotatable (e.g., manually) to cause rotation of the end effector assembly 4000 (without requiring actuation of the motor 154 to cause rotation of the second coupling shaft 64b, for instance).

With reference to FIGS. 23-30, the third force/rotation transmitting/converting assembly 2300 includes the third proximal drive shaft 2310, which, as described above, is rotatably supported within the proximal housing assembly 2004. The third proximal drive shaft 2310 includes a non-circular or shaped proximal end portion configured for connection with the third connector 2022 which is engageable with the third coupling shaft 64c of the surgical device 100. As discussed in further detail below, rotation of the third proximal drive shaft 2310 results in articulation of the end effector assembly 4000.

With particular reference to FIGS. 23, 25 and 28, the third force/rotation transmitting/converting assembly 2300 also includes an articulation pinion 2320, an articulation gear 2330, a drive tube 2340, a proximal cam 2350, a proximal pin 2355, a distal cam 2360, a distal pin 2365, an isolator tube 2370, a clocking ring 2375, the first articulation tube 2380, and the second articulation tube 2390. The core tube 2007 is also part of the third force/rotation transmitting/converting assembly 2300.

A distal portion 2312 of the third proximal drive shaft 2310 is engaged with or coupled to the articulation pinion 2320 of the third force/rotation transmitting/converting assembly 2300. The articulation pinion 2320 includes a plurality of gear teeth 2320a. The articulation gear 2330 is configured to rotate about the longitudinal axis “X-X” and includes a plurality of gear teeth 2330a. As shown in FIG. 25, the gear teeth 2320a of the articulation pinion 2320 are configured to mesh with the gear teeth 2330a of the articulation gear 2330. Thus, rotation of the articulation pinion 2320 about the third proximal drive shaft 2310 results in a corresponding rotation of the articulation gear 2330 about the longitudinal axis “X-X” relative to the knob housing 2002.

With particular reference to FIG. 25, the drive tube 2340 extends distally from the articulation gear 2330 and is rotationally fixed to the articulation gear 2330 by a pair of fingers 2332 extending radially inwardly from an inner wall 2334 of the articulation gear 2330 and into respective cavities 2342 defined with the drive tube 2340. The drive tube 2340 defines a channel 2344 through which at least portions of the proximal cam 2350 and the distal cam 2360 can longitudinally travel. The channel 2344 defined by the drive tube 2340 is co-axial with the longitudinal axis “X-X.” The rotational fixation between the articulation gear 2330 and the drive tube 2340 causes the drive tube 2340 to rotate relative to the knob housing 2002 as the articulation gear 2330 rotates relative to the knob housing 2002.

The isolator tube 2370 is disposed within the knob housing 2002 and radially outward of the drive tube 2340. The isolator tube 2370 is keyed to the core housing 2040 such that rotation of the core housing 2040 relative to the knob housing 2002 results in a corresponding rotation of the isolator tube 2370 relative to the knob housing 2002. The drive tube 2340 is rotatable relative to the isolator tube 2370. The proximal pin 2355 is affixed to the drive tube 2340, and engages the proximal cam 2350. The distal pin 2365 is affixed to the drive tube 2340, and engages the distal cam 2360. Additionally, the proximal pin 2355 and the distal pin 2365 are fixed from longitudinal movement relative to the knob housing 2002.

With particular reference to FIG. 23, the proximal cam 2350 and the distal cam 2360 are radially supported by the drive tube 2340. Additionally, the distal cam 2360 is at least partially radially supported by the clocking ring 2375 and a distal cam bushing 2372. Each of the proximal cam 2350 and the distal cam 2360 is positioned radially surrounding portions of the core tube 2007. Additionally, the proximal cam 2350 is longitudinally translatable within a portion of a channel 2361 defined by the distal cam 2360. The proximal cam 2350 includes an outer wall 2352 defining a threaded portion or groove 2354, and the distal cam 2360 includes an outer wall 2362 defining a threaded portion or groove 2364. The proximal pin 2355 extending from the drive tube 2340 engages the groove 2354 of the proximal cam 2350, and the distal pin 2365 extending from the drive tube 2340 engages the groove 2364 of the distal cam 2360.

Additionally, the isolator tube 2370 is rotationally fixed to the core housing 2040 and to the clocking ring 2376.

With reference to FIG. 25, a transverse cross-section of the channel 2344 of the drive tube 2340 defines a non-circle shape. In the illustrated embodiment, the cross-section includes two flat portions 2344a, 2344b, but other non-circle cross-sections are contemplated. Likewise, the proximal cam 2350 includes relief features to clear lugs added to the core housing 2040 (the lugs are included to help ensure a fixed rotational connection between the core tube 2007 and the core housing 2040), such that the transverse cross-section defines a similar or the same non-circle shape. In the embodiment shown in FIG. 25, the cross-section includes two flat portions 2358a, 2358b. The alignment of the flat portions 2358a, 2358b of the proximal cam 2350 with the flat portions on the core tube 2007 prevents rotation of the proximal cam 2350 relative to the drive tube 2340. Further, the core tube 2007 is rotationally fixed to the core housing 2040.

Referring to FIG. 23, the clocking ring 2375 radially surrounds a portion of the distal cam 2360. The clocking ring 2375 includes a first finger 2376 and a second finger 2378. The clocking ring 2375, in connection with the isolator tube 2070, rotationally fixes the distal cam 2360 to the core housing 2040.

During rotation of the proximal pin 2355 and the distal pin 2365 relative to the core housing 2040, the proximal pin 2355 travels through the groove 2354 of the proximal cam 2350, and the distal pin 2365 travels through the groove 2364 of the distal cam 2360. Additionally, since the proximal cam 2350 and the distal cam 2360 are fixed from rotation relative to the core housing 2040, the travel of the proximal pin 2355 through the groove 2354 of the proximal cam 2350 causes the proximal cam 2350 to move longitudinally (proximally or distally) relative to the core housing 2040, and the travel of the distal pin 2365 through the groove 2364 of the distal cam 2360 causes the distal cam 2360 to move longitudinally (proximally or distally) relative to the knob housing 2002. Further, the groove 2354 of the proximal cam 2350 is oriented in a first thread direction, and the groove 2364 of the distal cam 2360 is orientated in a second, opposite thread direction. Thus, rotation of the drive tube 2340, the proximal pin 2355, and the distal pin 2365 in a first direction (e.g., clockwise) causes the proximal cam 2350 to move in a first direction (e.g., proximally) and causes the distal cam 2360 to move in a second, opposite direction (e.g., distally), and rotation of the drive tube 2340, the proximal pin 2355, and the distal pin 2365 in a second direction (e.g., counter-clockwise) causes the proximal cam 2350 to move in the second direction (e.g., distally) and causes the distal cam 2360 to move in the first direction (e.g., proximally).

With reference to FIG. 23, proximal portions of the first articulation tube 2380, and the second articulation tube 2390 are shown. The first articulation tube 2380 is disposed radially inward of the outer tuber 2006, radially outward of the core tube 2007, and in mechanical cooperation with the proximal cam 2350. The second articulation tube 2390 is disposed radially inward of the outer tuber 2006, radially outward of the first articulation tube 2380, and in mechanical cooperation with the distal cam 2360.

With continued reference to FIG. 23, a proximal leg 2382 of the first articulation tube 2380 is supported in an annular groove 2353 of the proximal cam 2350. A proximal leg 2392 of the second articulation tube 2390 is supported in an annular groove 2363 of the distal cam 2360. The proximal cam 2350 is rotatable relative to the first articulation tube 2380, and the distal cam 2360 is rotatable relative to the second articulation tube 2390. Further, the first articulation tube 2380 is fixed from longitudinal movement relative to the proximal cam 2350, and the second articulation tube 2390 is fixed from longitudinal movement relative to the distal cam 2360.

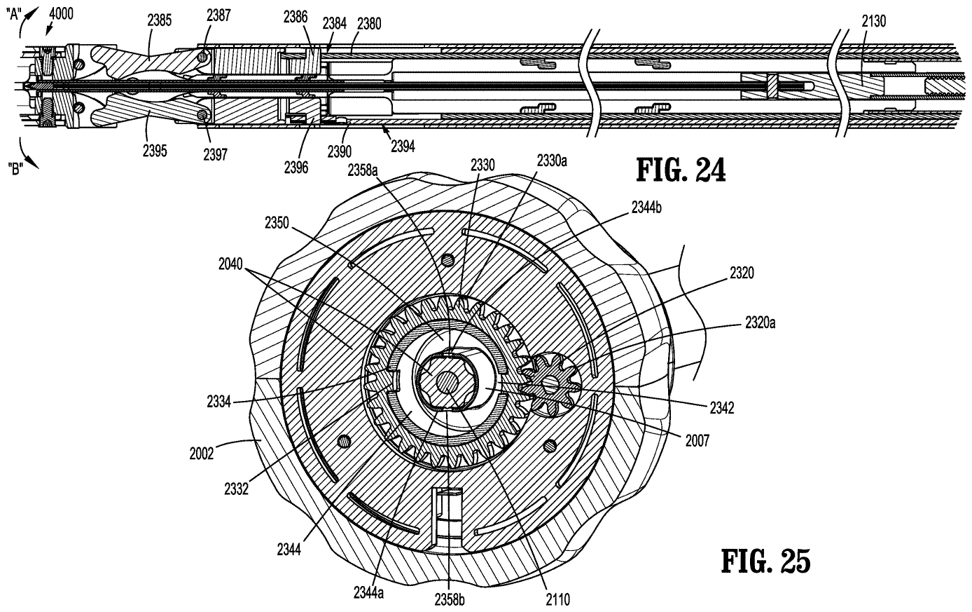

Referring now to FIG. 24, distal portions 2384, 2394 of the first articulation tube 2380 and the second articulation tube 2390, respectively, are shown. Generally, the distal portion 2384 of the first articulation tube 2380 is mechanically engaged with a first pivot link 2385, and the distal portion 2394 of the second articulation tube 2390 is mechanically engaged with a second pivot link 2395. The first pivot link 2385 is coupled to a first lateral side of the end effector assembly 4000, and the second pivot link 2395 is coupled to a second lateral side of the end effector assembly 4000. More particularly, the distal portion 2384 of the first articulation tube 2380 is pivotably coupled to the first pivot link 2385 via a first distal linkage 2386 about a proximal or first pivot link pin 2387, and the distal portion 2394 of the second articulation tube 2390 is pivotably coupled to the second pivot link 2395 via a second distal linkage 2396 about a proximal or second pivot link pin 2397.

Thus, as the first articulation tube 2380 translates proximally, the distal portion 2384 of the first articulation tube 2380 and the first distal linkage 2386 also move proximally causing the first pivot link 2385 to pivot about the first pivot link pin 2387 which articulates (or pulls) the end effector assembly 4000 in the general direction of arrow “A” in FIG. 24. Additionally, while the first articulation tube 2380 translates proximally, the second articulation tube 2390 translates distally. The distal translation of the second articulation tube 2390 causes the distal portion 2394 thereof and the second distal linkage 2396 to also move distally. This causes the second pivot link 2395 to pivot about the second pivot link pin 2397 which also articulates (or pushes) the end effector in the general direction of arrow “A” in FIG. 24. Thus, the end effector assembly 4000 is simultaneously being articulated or moved in the same direction by proximal translation of the first articulation tube 2380 and by distal translation of the second articulation tube 2390. Likewise, distal translation of the fist articulation tube 2380 and proximal translation of the second articulation tube 2390 would both cause the end effector assembly 4000 to articulate or move in the general direction of arrow “B” in FIG. 24, which is opposite from the general direction of arrow “A.”

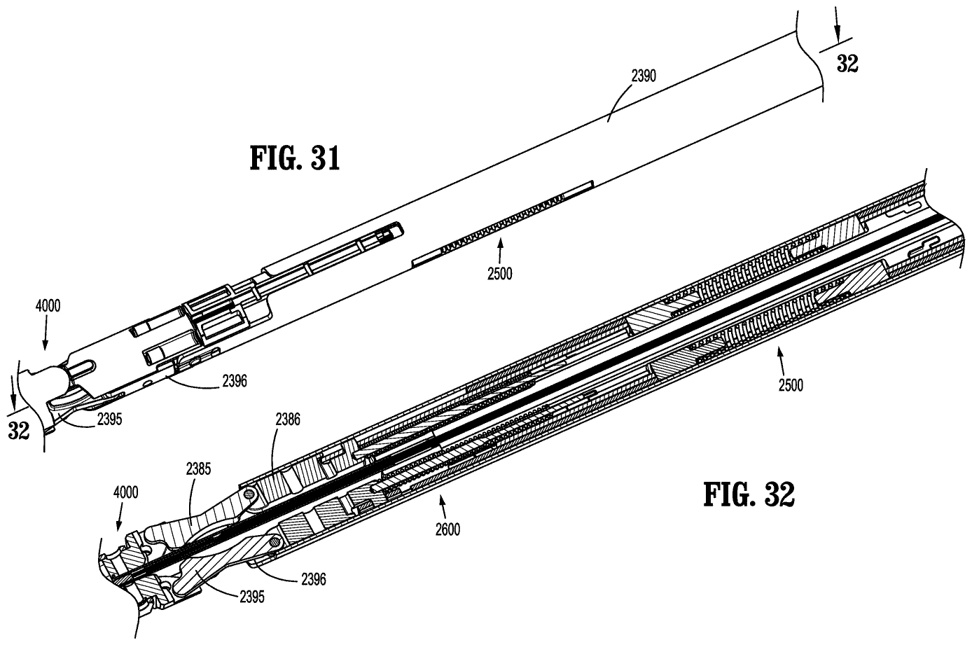

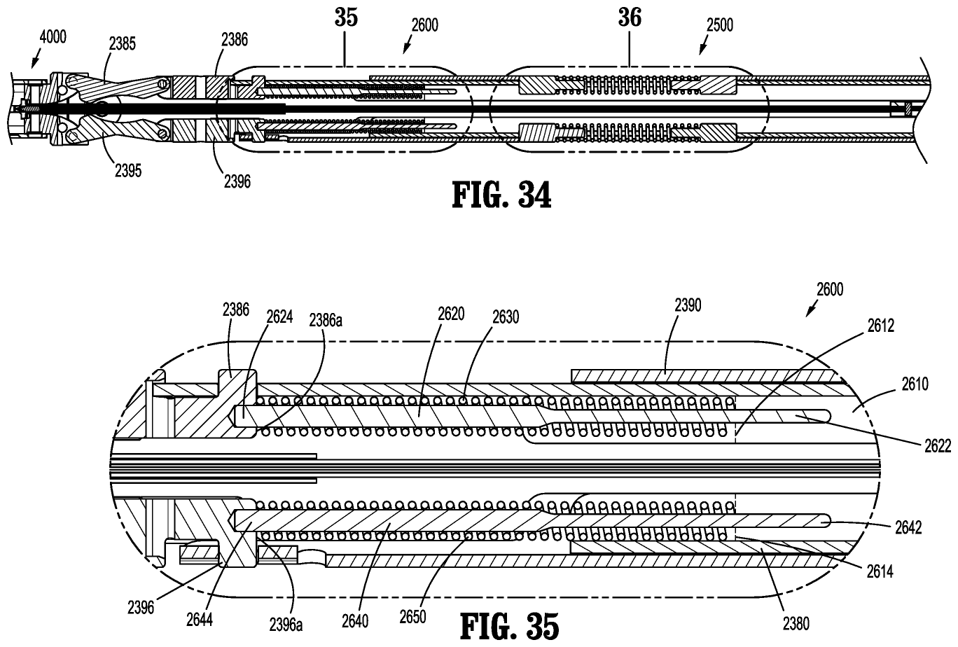

With reference to FIGS. 31-39, a first spring-loaded articulation assembly 2500 and a second spring-loaded articulation assembly 2600 are shown. The first spring-loaded articulation assembly 2500 is disposed within a distal portion of the outer tube 2006 and is engaged with the first articulation tube 2380 and the second articulation tube 2390.

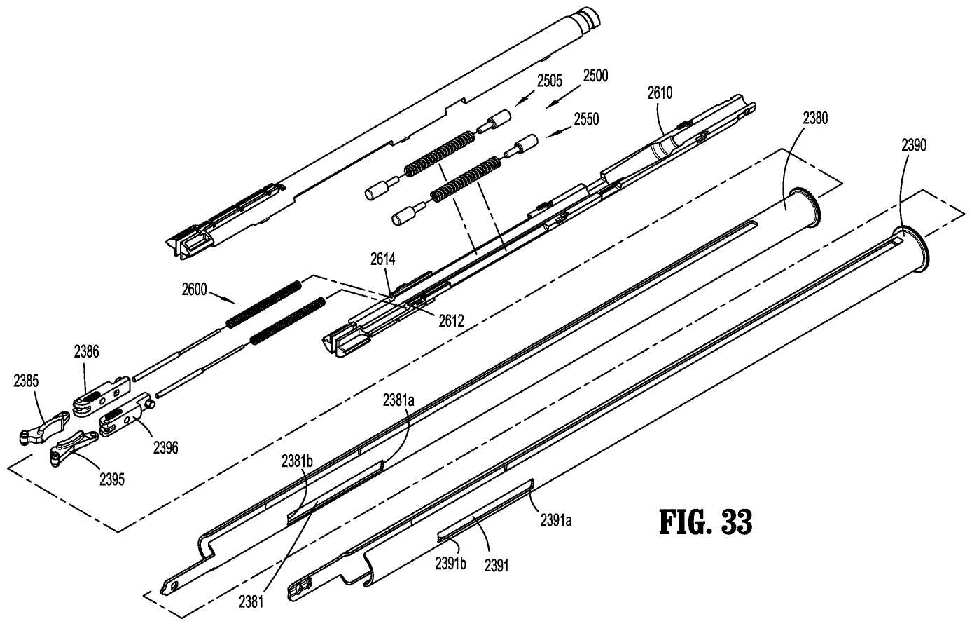

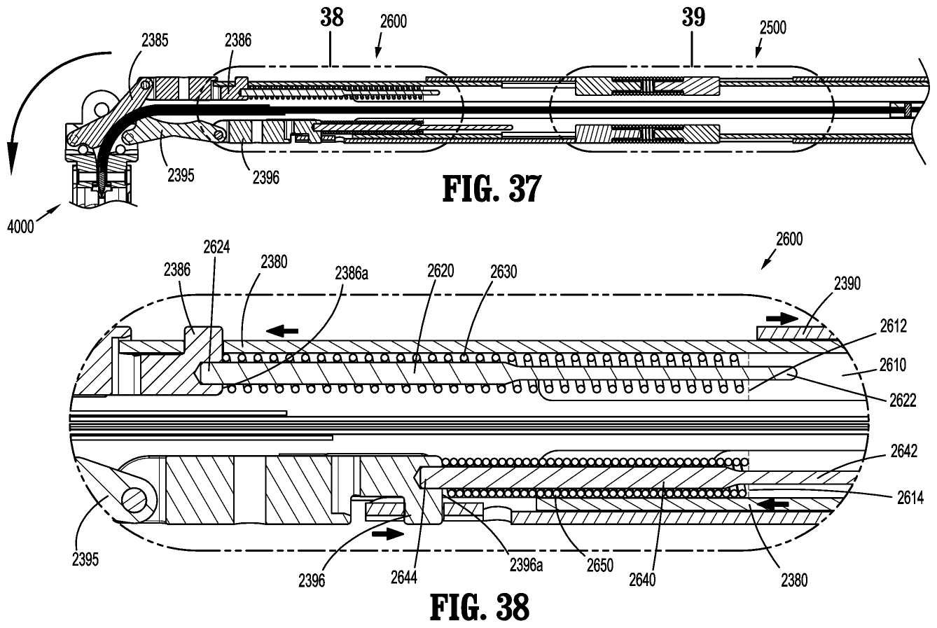

With particular reference to FIGS. 33, 36 and 39, the first spring-loaded articulation assembly 2500 includes a first spring-loaded articulation mechanism 2505 and a second spring-loaded articulation mechanism 2550. The first spring-loaded articulation mechanism 2505 includes a first proximal boss 2510, a first distal boss 2520, and a first biasing member 2530. The second spring-loaded articulation mechanism 2550 includes a second proximal boss 2560, a second distal boss 2570, and a second biasing member 2580. As shown in FIG. 37, the first spring-loaded articulation mechanism 2505 and the second spring-loaded articulation mechanism 2550 are generally aligned longitudinally and are disposed on opposing sides of the longitudinal axis “X-X” (when the first articulation tube 2380 and the second articulation tube 2390 are in a neutral position).

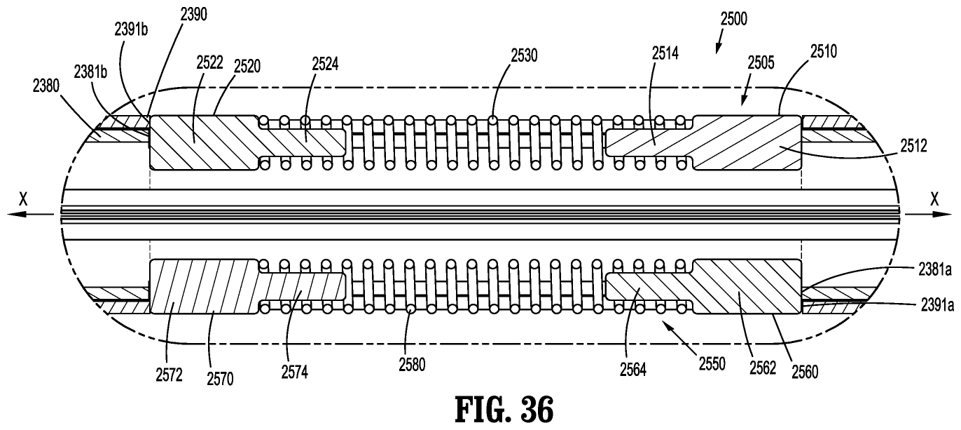

With particular reference to FIG. 36, when the first articulation tube 2380 and the second articulation tube 2390 are in the neutral position, the first proximal boss 2510 of the first spring-loaded articulation mechanism 2505 and the second proximal boss 2560 of the second spring-loaded articulation mechanism 2550 are each positioned in contact with a proximal face 2381a of a recess 2381 defined by the first articulation tube 2380 and in contact with a proximal face 2391a of a recess 2391 defined by the second articulation tube 2390. Additionally, in this position, the first distal boss 2520 of the first spring-loaded articulation mechanism 2505 and the second distal boss 2570 of the second spring-loaded articulation mechanism 2550 are each positioned in contact with a distal face 2381b of the recess 2381 of the first articulation tube 2380 and in contact with a distal face 2391b of the recess 2391 defined by the second articulation tube 2390.

The first proximal boss 2510 includes a body portion 2512 and a finger 2514 extending distally from the body portion 2512. The first distal boss 2520 includes a body portion 2522 and a finger 2524 extending proximally from the body portion 2522. The second proximal boss 2560 includes a body portion 2562 and a finger 2564 extending distally from the body portion 2562. The second distal boss 2570 includes a body portion 2572 and a finger 2574 extending proximally from the body portion 2572.

The first biasing member 2530 of the first spring-loaded articulation mechanism 2505 extends between the first proximal boss 2510 and the first distal boss 2520. More particularly, the first biasing member 2530 (e.g., a coil spring) radially surrounds the finger 2514 of the first proximal boss 2510, and radially surrounds the finger 2524 of the first distal boss 2520. The first biasing member 2530 biases the first proximal boss 2510 and the first distal boss 2520 away from each other, thereby biasing the first articulation tube 2380 and the second articulation tube 2390 towards their neutral position (shown in FIG. 36).

The second biasing member 2580 of the second spring-loaded articulation mechanism 2550 extends between the second proximal boss 2560 and the second distal boss 2570. More particularly, the second biasing member 2580 (e.g., a coil spring) radially surrounds the finger 2564 of the second proximal boss 2560, and radially surrounds the finger 2574 of the second distal boss 2570. The second biasing member 2580 biases the second proximal boss 2560 and the second distal boss 2570 away from each other, thereby biasing the first articulation tube 2380 and the second articulation tube 2390 towards their neutral position (shown in FIG. 36).

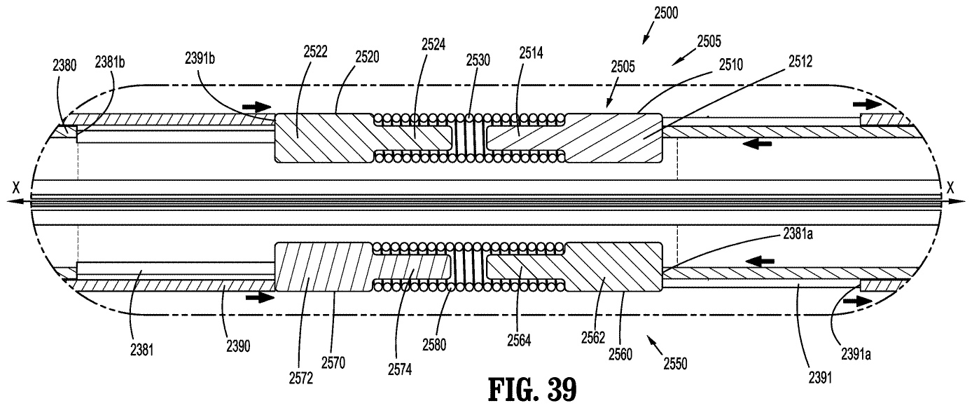

As discussed herein, during articulation of the end effector assembly 4000, as the first articulation tube 2380 moves in a first longitudinal direction (e.g., proximally), the second articulation tube 2390 moves in a second, opposite longitudinal direction (e.g., distally). The proximal movement of the first articulation tube 2380 causes the distal face 2381b of the recess 2381 defined by the first articulation tube 2380 to move proximally, while the distal movement of the second articulation tube 2390 causes the proximal face 2391a of the recess 2391 defined by the second articulation tube 2390 to move distally, thereby compressing both the first biasing member 2530 and the second biasing member 2580. Likewise, as shown in FIG. 39, distal movement of the first articulation tube 2380 and proximal movement of the second articulation tube 2390 also result in compressing both the first biasing member 2530 and the second biasing member 2580.

The forces provided by the first biasing member 2530 and the second biasing member 2580 result in corresponding distally-directed forces exerted on the first pivot link 2385 and the second pivot link 2395. These forces help maintain the end effector assembly 4000 is a desired articulated position by minimizing movement of the end effector assembly 4000 during utilization of the surgical device 100 (e.g., during firing or retraction motion of a drive beam assembly 4350) which otherwise may occur due to various working loads, clearances, and tolerances.

Referring now to FIGS. 33, 35 and 38, the second spring-loaded articulation assembly 2600 is disposed within a distal portion of the outer tube 2006 and is directly engaged with the first distal linkage 2386 and the second distal linkage 2396. The second spring-loaded articulation assembly 2600 includes a first support rod 2620 supporting a first biasing member 2630 (e.g., a coil spring), and a second support rod 2640 supporting a second biasing member 2650 (e.g., a coil spring).

With particular reference to FIGS. 35 and 38, a proximal portion 2622 of the first support rod 2620 and a proximal portion 2642 of the second support rod 2640 extend distally from and are secured to a support structure 2610 within the outer tube 2006. A distal portion 2624 of the first support rod 2620 is secured to the first distal linkage 2386, and a distal portion 2644 of the second support rod 2640 is secured to the second distal linkage 2396. The first biasing member 2630 radially surrounds a portion of the first support rod 2620 and is positioned between a first spring stop 2612 of the support structure 2610 and a proximal face 2386a of the first distal linkage 2386. The second biasing member 2650 radially surrounds a portion of the second support rod 2640 and is positioned between a second spring stop 2614 of the support structure 2610 and a proximal face 2396a of the second distal linkage 2396. The support structure 2610 is longitudinally fixed relative to the outer tube 2006.

Referring now to FIG. 38, during articulation of the end effector assembly 4000, as the first articulation tube 2380 moves in a first longitudinal direction (e.g., distally), the second articulation tube 2390 moves in a second, opposite longitudinal direction (e.g., proximally). The distal movement of the first articulation tube 2380 causes the first distal linkage 2386 to move distally, thereby allowing the first biasing member 2630 to move toward an expanded orientation. The proximal movement of the second articulation tube 2390 causes the second distal linkage 2396 to move proximally, thereby forcing the second biasing member 2650 to move toward a compressed orientation. Additionally, during articulation of the end effector assembly 4000 in the opposite direction, distal movement of the second articulation tube 2390 causes the second biasing member 2650 to move toward an expanded orientation, and proximal movement of the first articulation tube 2380 causes the first biasing member 2630 to move toward a compressed orientation.

Accordingly, the first biasing member 2630 biases the first distal linkage 2386 in a first direction relative to the outer tube 2006, and the second biasing member 2650 biases the second distal linkage 2396 in a second, opposite direction relative to the outer tube 2006. These forces help maintain the end effector assembly 4000 is a desired articulated position by minimizing movement of the end effector assembly 4000 during utilization of the surgical device 100 (e.g., during firing or retraction motion of the drive beam assembly 4350) which otherwise may occur due to various working loads, clearances, and tolerances.

The first spring-loaded articulation assembly 2500 and the second spring-loaded articulation assembly 2600 are usable individually or in combination with each other.

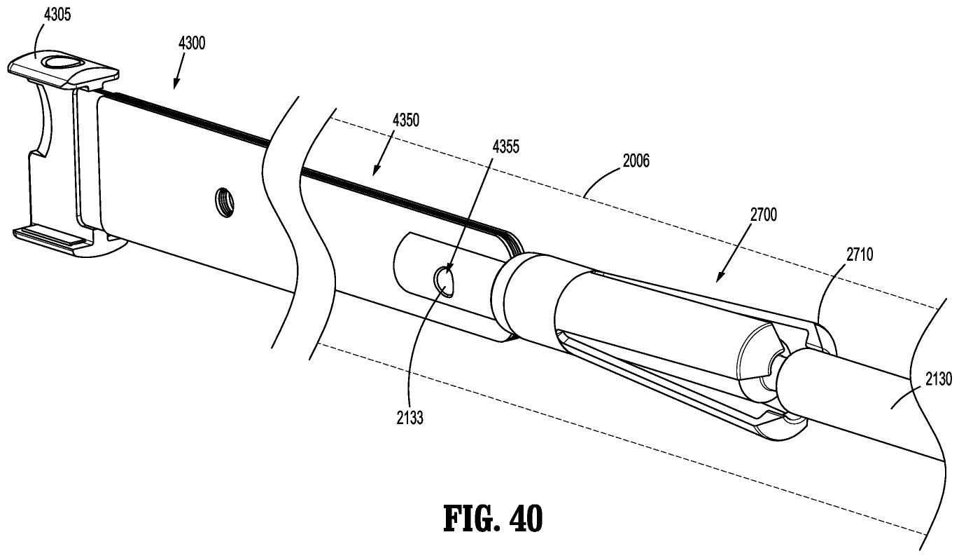

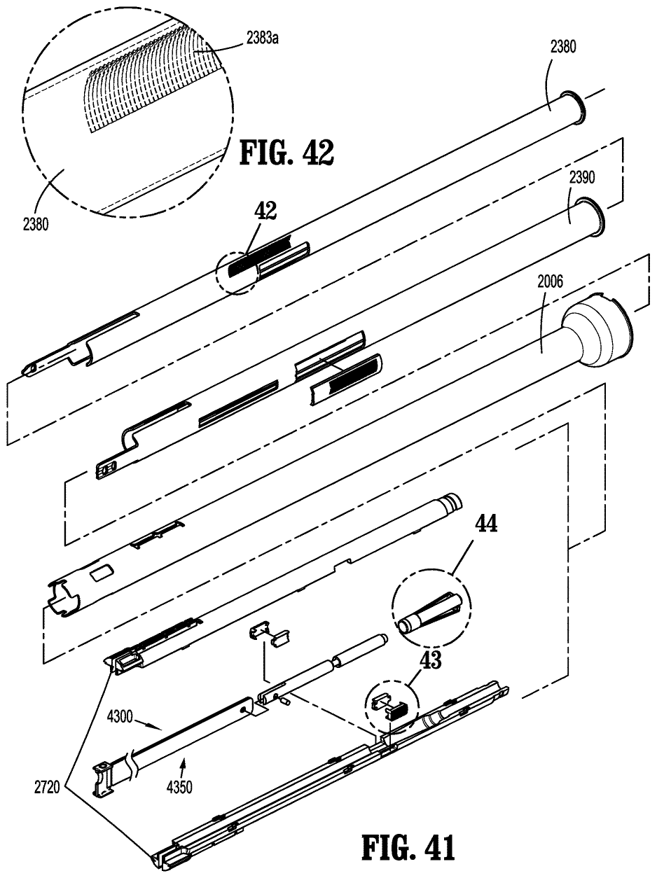

Referring now to FIGS. 40-49, an articulation locking assembly 2700 is shown. The articulation locking assembly 2700 is disposed within the outer tube 2006 and is configured to mechanically engage the firing rod assembly 2130, the first articulation tube 2380, the second articulation tube 2390, and a drive beam assembly 4350. The articulation locking assembly 2700 is configured to prevent longitudinal translation of the first articulation tube 2380 and the second articulation tube 2390, and any resulting articulation (e.g., unintentional articulation) of the end effector assembly 4000, during approximation of the cartridge assembly 4120 and the anvil assembly 4140, and during the ejection of fasteners from the cartridge assembly 4120.

With continued reference to FIGS. 40-49, the articulation locking assembly 2700 includes a collet 2710, a tube adapter 2720, a first brake 2730, and a second brake 2740. Generally, the collet 2710 engages with the firing rod assembly 2130 and the tube adapter 2720 to move the first brake 2730 and the second brake 2740 into and out of engagement with the first articulation tube 2380 and the second articulation tube 2390, respectively, during longitudinal movement of the firing rod assembly 2130.

With particular reference to FIG. 44, the collet 2710 includes a body portion or cylindrical portion 2714, a plurality of legs 2713 extending proximally from the cylindrical portion 2714, and a proximal tapered portion 2712 on a proximal end of each leg of the plurality of legs 2713. The tapered portion 2712 and the plurality of legs 2713 are biased radially outward.

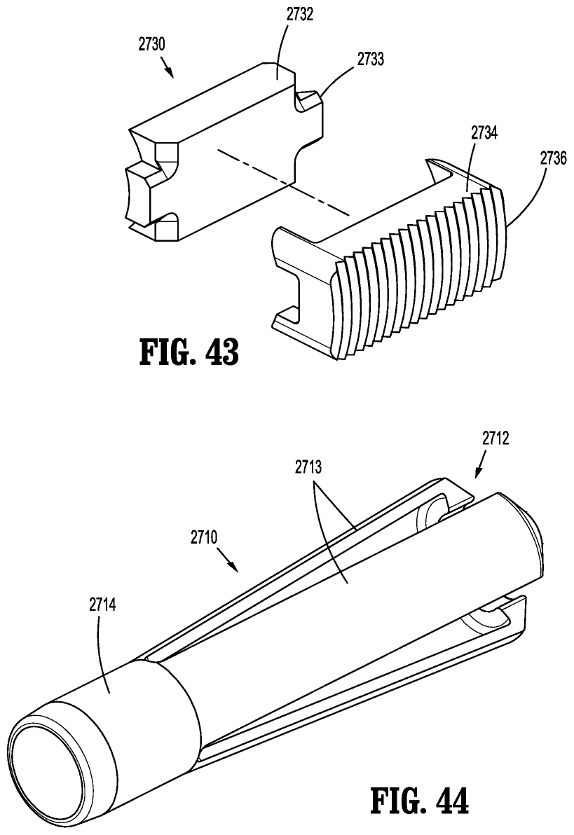

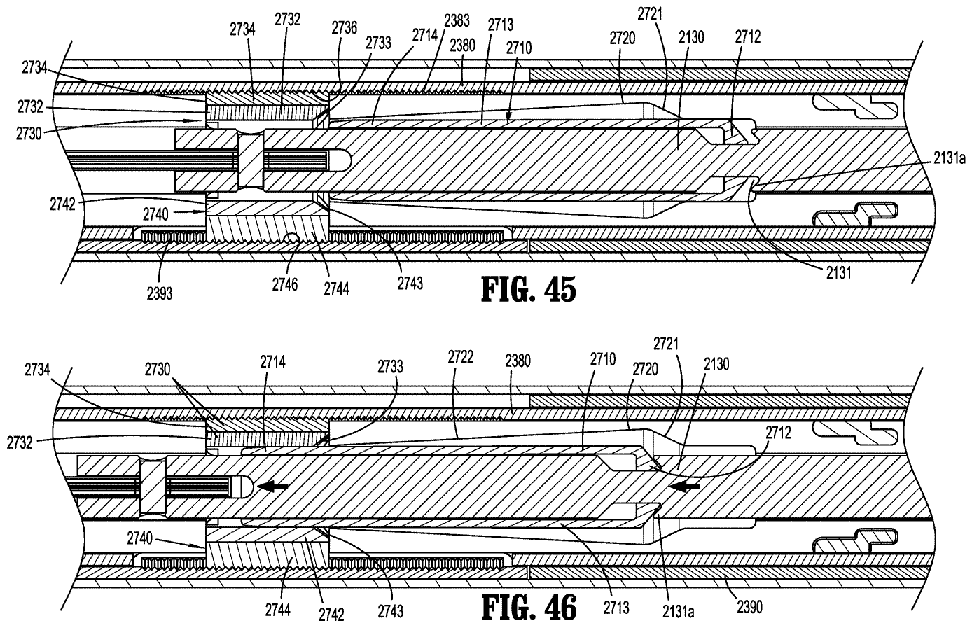

Referring to FIGS. 45-49, the tube adapter 2720 is disposed within the outer tube 2006, and radially inward of the first articulation tube 2380 and the second articulation tube 2390. The tube adapter 2720 is fixed from longitudinal movement relative to the outer tube 2006, and includes a proximal angled portion 2721 and a distal angled portion 2722.

With reference to FIGS. 43 and 45-49, the first brake 2730 and the second brake 2740 are disposed within the outer tube 2006 and are positioned distally of the distal angled portion 2722 of the tube adapter 2720. The first brake 2730 includes an inner portion 2732 configured to engage the cylindrical portion 2714 of the collet 2710, and an outer portion 2734 configured to engage the first articulation tube 2380. The second brake 2740 includes an inner portion 2742 configured to engage the cylindrical portion 2714 of the collet 2710, and an outer portion 2744 configured to engage the second articulation tube 2390. A proximal end 2733 of the inner portion 2732 of the first brake 2730 is angled to direct a distal portion of the collet 2710 radially inward, and a proximal end 2743 of the inner portion 2742 of the second brake 2740 is angled to direct a distal portion of the collet 2710 radially inward.

Further, the outer portion 2734 of the first brake 2730 includes teeth 2736 (or other friction-enhancing features) configured to engage corresponding teeth 2383 (or other friction-enhancing features 2383a (FIG. 42), for instance) of the first articulation tube 2380. The outer portion 2744 of the second brake 2740 includes teeth 2746 (or other friction-enhancing features) configured to engage corresponding teeth 2393 (or other friction-enhancing features) of the second articulation tube 2390.

Moreover, the first brake 2730 and the second brake 2740 are rotationally coupled to the first articulation tube 2380 and the second articulation tube 2390 via the tube adapter 2720.

In embodiments, the inner portion 2732 of the first brake 2730 and the inner portion 2742 of the second brake 2740 are made from a first material (e.g., metal, rubber, etc.), and the outer portion 2734 of the first brake 2730 and the outer portion 2744 of the second brake 2740 are made from a second, different material (e.g., plastic). In other embodiments, the entirety of the first brake 2730 and the entirety of the second brake 2740 are made from the same material.

In use, the collet 2710 is configured to move proximally and distally relative to the tube adapter 2720 in response to longitudinal movement of the firing rod assembly 2130. FIG. 45 illustrates the articulation locking assembly 2700 in an initial position corresponding to the firing rod assembly 2130 being in a proximal or retracted position. Here, a groove 2131 (e.g., an annular groove) of the firing rod assembly 2130 is disposed proximally of the tapered portion 2712 of the collet 2710, and the cylindrical portion 2714 of the collet 2710 is disposed proximally of the first brake 2730 and the second brake 2740. Here, the first brake 2730 and the second brake 2740 are not being urged into engagement with the first articulation tube 2380 or the second articulation tube 2390, respectively. Accordingly, when the firing rod assembly 2130 is in this proximal position, the first articulation tube 2380 and the second articulation tube 2390 are free to move longitudinally thereby articulating the end effector assembly 4000.

FIG. 46 illustrates the articulation locking assembly 2700 in a second position, corresponding to the firing rod assembly 2130 in a partially advanced position (e.g., when the cartridge assembly 4120 and an anvil assembly 4140 are in an approximated or clamped position). Here, the distal translation of the firing rod assembly 2130 has resulted in a finger 2131a (e.g., an annular finger) of the firing rod assembly 2130 contacting the tapered portion 2712 of the collet 2710 and forcing the collet 2710 distally. Additionally, the engagement between the finger 2131a and the tapered portion 2712 of the collet 2710 helps maintain the proximal portion of the collet 2710 in its radial position. During distal movement of the collet 2710, the proximal end 2733 of the first brake 2730, and the proximal end 2743 of the second brake 2740 urge, direct or wedge the distal part of the cylindrical portion 2714 of the collet 2710 into engagement with the first brake 2730, and into engagement with the second brake 2740, respectively. In this position, the first brake 2730 moves radially outward into engagement (or into a stronger engagement) with the first articulation tube 2380, and the second brake 2740 moves radially outward into engagement (or into a stronger engagement) with the second articulation tube 2390. Here, the first articulation tube 2380 and the second articulation tube 2390 are prevented or hindered from moving longitudinally relative to the outer tube 2006 thereby effectively locking the articulated position of the end effector assembly 4000. Additionally, the firing rod assembly 2130 is in radial clearance with the collet 2710, thereby allowing longitudinal translation of the firing rod assembly 2130 relative to the collet 2710.

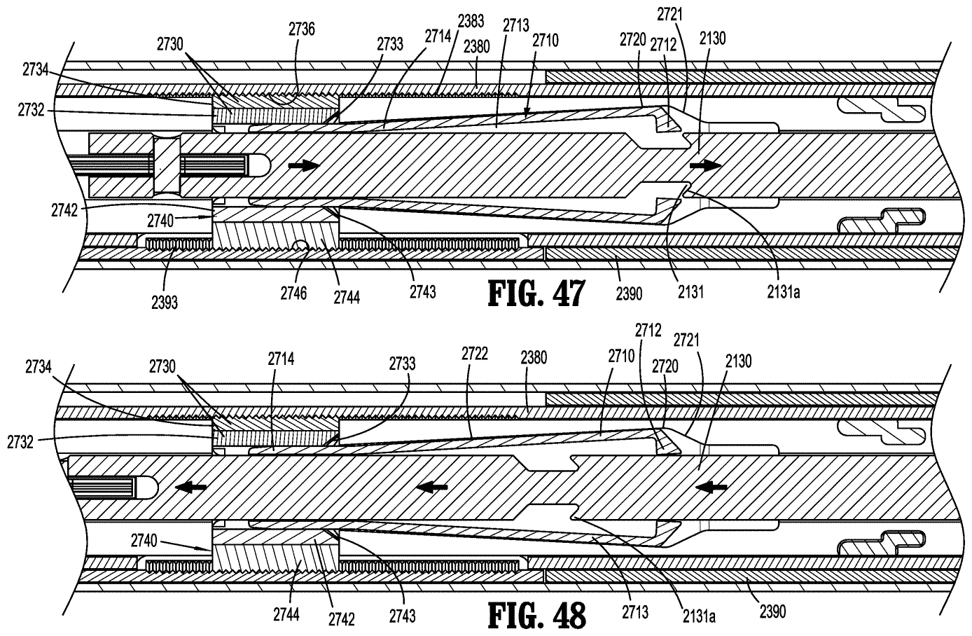

Referring now to FIG. 47, the third position of the articulation locking assembly 2700 is shown, which corresponds to the firing rod assembly 2130 being partially retracted from its second position. In embodiments, the gearing associated with longitudinal translation of the firing rod assembly 2130 is designed to provide for a sufficient retraction of the firing rod assembly 2130 during its firing stroke. In this position, the firing rod assembly 2130 has been retracted a sufficient distance relative to the collet 2710 such that the tapered portion 2712 of the collet 2710 is free from engagement with the groove 2131 of the firing rod assembly 2130, thereby permitting proximal portions of the plurality of legs 2713 of the collet 2710 to move radially outward (i.e., in the biased direction). The distal angled portion 2722 of the tube adapter 2720 helps create a cavity into which portions of the collet 2710 can expand. Additionally, in this position, the distal portion of the cylindrical portion 2714 of the collet 2710 remains wedged in engagement with the first brake 2730 and the second brake 2740, thereby maintaining the locked, articulated position of the end effector assembly 4000.

Referring now to FIG. 48, the fourth position of the articulation locking assembly 2700 is shown, which corresponds to the firing rod assembly 2130 being in or near its distal-most position. In this position, the full translation of the firing rod assembly 2130 indicates that fasteners have been fired from the cartridge assembly 4120. Here, the finger 2131a of the firing rod assembly 2130 has been translated distally beyond the tapered portion 2712 of the collet 2710. (The radial outward movement of the tapered portion 2712 of the collet 2710 shown in FIG. 47 effectively moves the tapered portion 2712 out of the way of the path of the firing rod assembly 2130 thereby allowing the distal translation of the firing rod assembly 2130.) Additionally, in this position, the distal portion of the cylindrical portion 2714 of the collet 2710 remains in engagement with the first brake 2730 and the second brake 2740, thereby maintaining the locked, articulated position of the end effector assembly 4000 during firing of the fasteners.

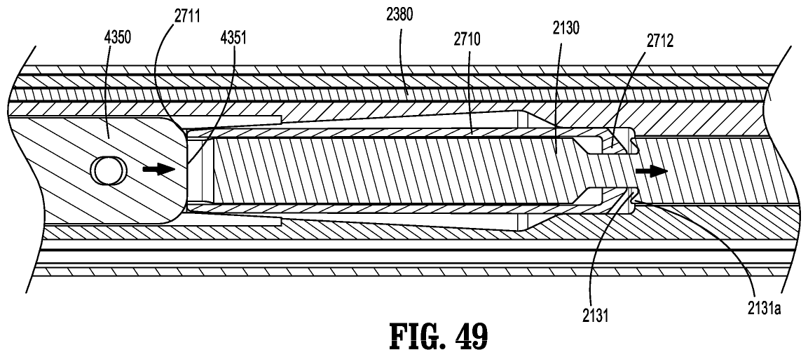

Retraction of the firing rod assembly 2130 is shown in FIG. 49. As discussed below, an actuation assembly or I-beam assembly 4300 is mechanically coupled to a distal end of the firing rod assembly 2130. Here, as the firing rod assembly 2130 and the I-beam assembly 4300 are retracted, a proximal face 4351 of the drive beam assembly 4350 of the I-beam assembly 4300 contacts a distal end 2711 of the collet 2710 and urges the collet 2710 proximally. More particularly, the collet 2710 is moved to a location that is proximal of the first brake 2730 and the second brake 2740, thereby allowing the first brake 2730 to move radially inward and out of engagement with the first articulation tube 2380, and allowing the second brake 2740 to move radially inward and out of engagement with the second articulation tube 2390 (the first brake 2730 and the second brake 2740 are not visible in FIG. 49).

Additionally, as the collet 2710 moves proximally, the tapered portion 2712 of the collet 2710 engages the proximal angled portion 2721 of the tube adapter 2720, which causes the tapered portion 2712 to move radially inward. Continued proximal movement of the firing rod assembly 2130 causes the collet 2710 to move to its initial, proximal position where the tapered portion 2712 of the collet 2710 engages the groove 2131 of the firing rod assembly 2130.

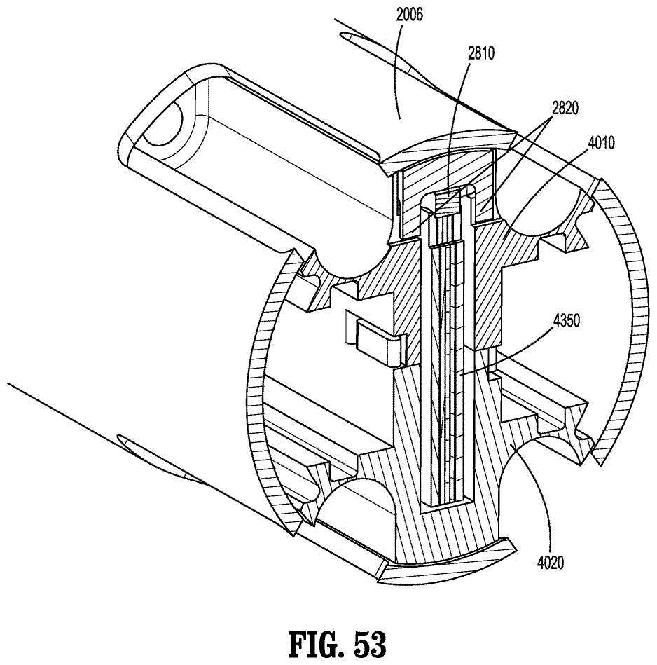

Referring now to FIGS. 50-56, an articulation control mechanism 2800 is shown. The articulation control mechanism 2800 includes a lock 2810, a block 2820, and a plurality of notches 2860 defined within a pivot assembly 2850. Generally, as the firing rod assembly 2130 moves distally, the drive beam assembly 4350 of the I-beam assembly 4300 moves a portion of the lock 2810 distally into engagement with a notch of the plurality of notches 2860, thereby locking the end effector assembly 4000 in its articulated position, and inhibiting or reducing change in the articulation angle during firing or retraction, for instance.

More particularly, and with reference to FIGS. 50-51, the lock 2810 and the block 2820 are disposed within the outer tube 2006 (a distal finger 2812 of the lock 2810 may extend distally of the outer tube 2006 during use). The plurality of notches 2860 of the pivot assembly 2850 is disposed distally of the outer tube 2006 and is part of the end effector assembly 4000. While nine notches 2860 are shown, the pivot assembly 2850 may include more or fewer notches 2860 without departing from the scope of the disclosure. Moreover, the number of notches 2860 of the pivot assembly 2850 determines the number of discrete articulated positions in which the end effector assembly 4000 can be locked.

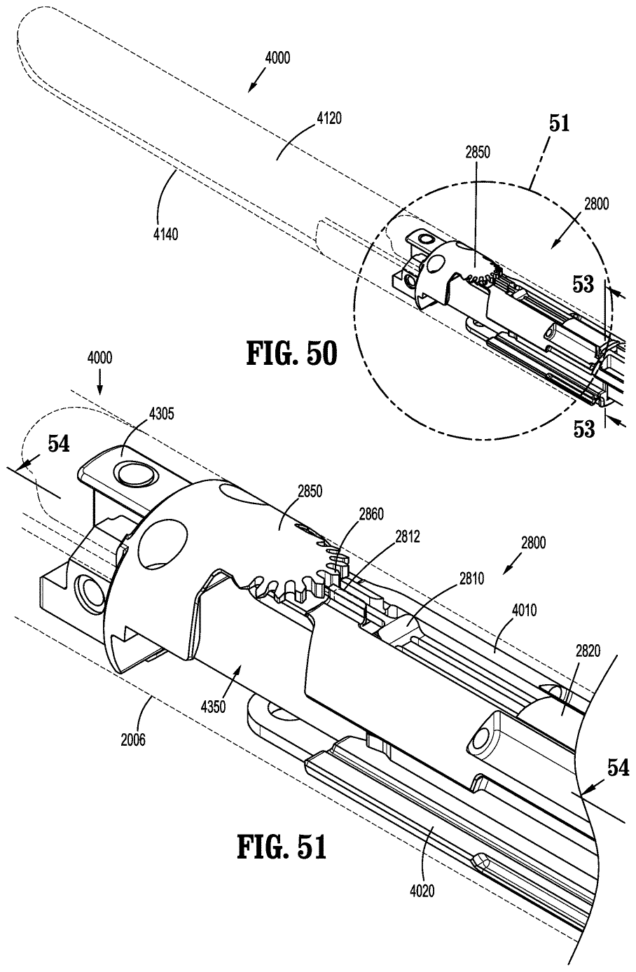

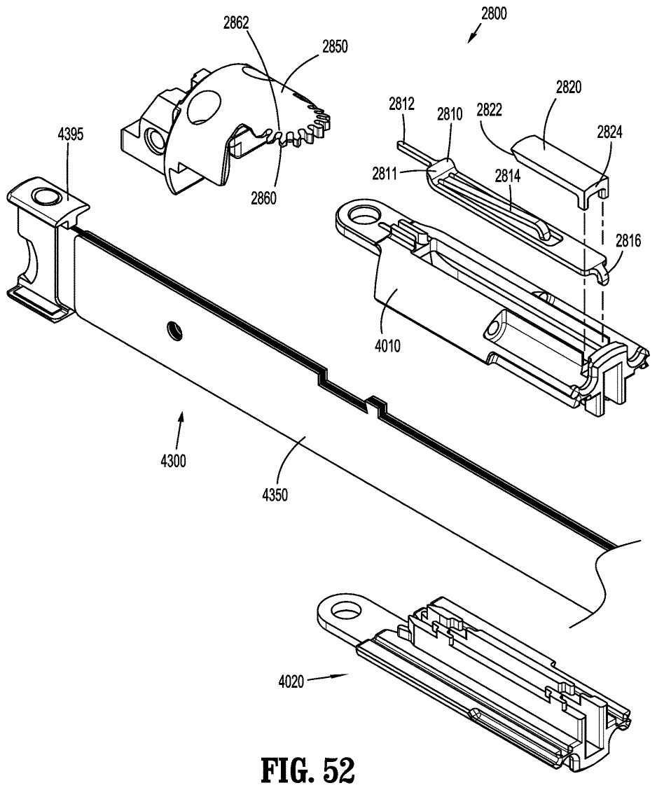

As shown in FIG. 52, the lock 2810 includes a body portion 2811, the distal finger 2812, a first leg 2814, and a second leg 2816. The distal finger 2812 extends distally from the body portion 2811 and is configured for selective engagement with the plurality of notches 2860 (one notch at a time) of the pivot assembly 2850. Moreover, in the illustrated embodiment, the geometry of the distal finger 2812 closely mirrors the geometry of an inner portion 2862 of each notch of the plurality of notches 2860 to create limited clearance therebetween and to help minimize movement of the end effector assembly 4000 when engaged, for instance.

The first leg 2814 of the lock 2810 extends proximally from the body portion 2811 and is configured to selectively engage the drive beam assembly 4350 and a distal ramp 2822 of the block 2820. Additionally, the first leg 2814 is biased radially outward away from the longitudinal axis “X-X.” The second leg 2816 of the lock 2810 extends proximally from the body portion 2811, is longer than the first leg 2814, and is configured to selectively engage the drive beam assembly 4350 and a proximal wall 2824 of the block 2820.

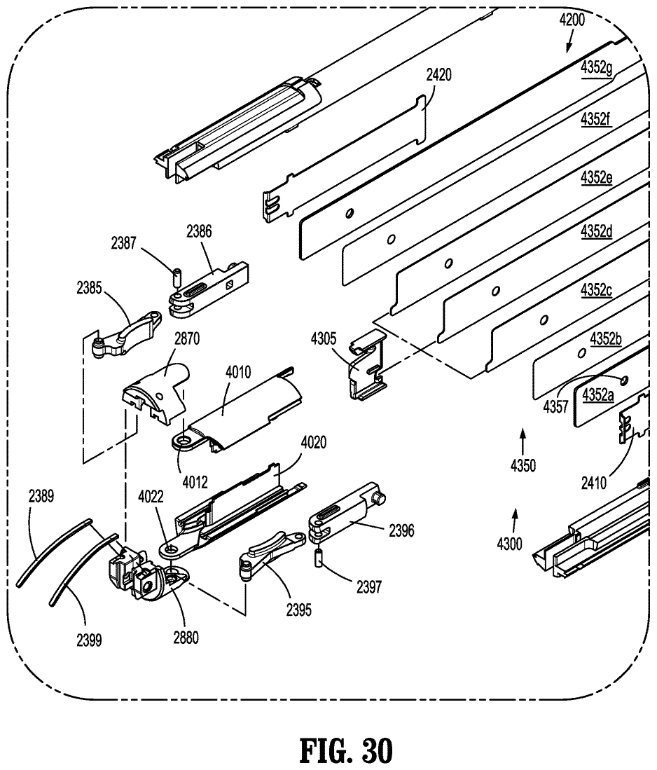

The block 2820 is keyed to a first tip adapter 4010 (see FIG. 30) and is maintained in its radial position by the outer tube 2006. The block 2820 includes the distal ramp 2822 and the proximal wall 2824, and is configured to direct and limit the travel of portions of the lock 2810.

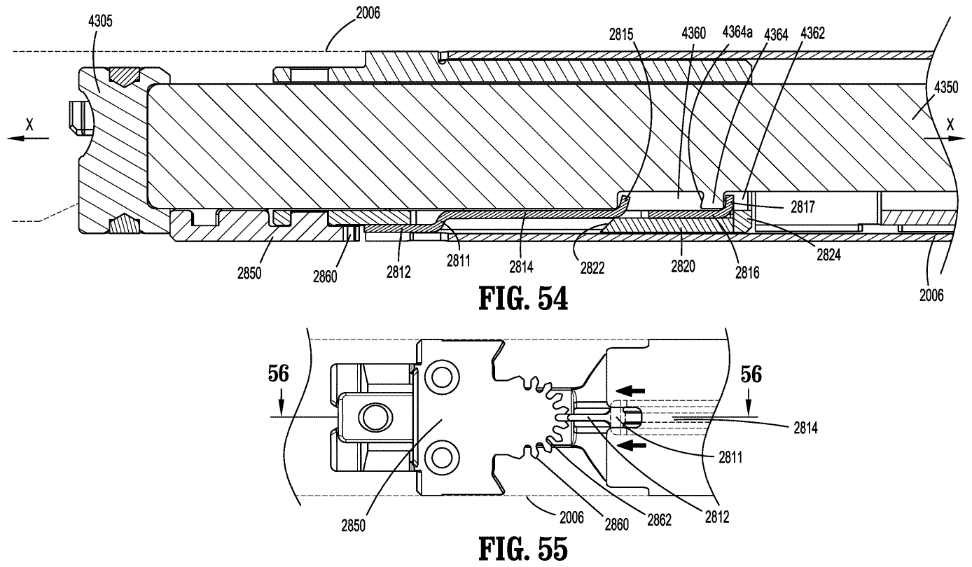

With particular reference to FIG. 54, in use, prior to actuation of the firing rod assembly 2130, the lock 2810 of the articulation mechanism 2800 is in its proximal position where a foot 2815 of the first leg 2814 of the lock 2810 is positioned at least partially within a first recess 4360 of the drive beam assembly 4350, and a foot 2817 of the second leg 2816 is positioned at least partially within a second recess 4362 of the drive beam assembly 4350. A tab 4364 of the drive beam assembly 4350 extends between the first recess 4360 and the second recess 4362.

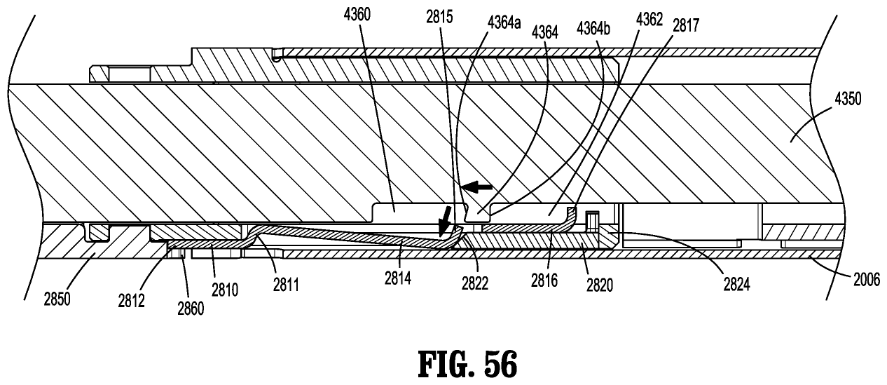

When the firing rod assembly 2130 moves distally (e.g., to approximate the cartridge assembly 4120 and the anvil assembly 4140, and/or to eject fasteners from the cartridge assembly 4120), the I-beam assembly 4300 (including the drive beam assembly 4350) also moves distally. Referring to FIG. 56, as the drive beam assembly 4350 moves distally, a distal face 4364a of the tab 4364 contacts the foot 2815 of the first leg 2814 causing the lock 2810 to move distally. A sufficient distal movement of the lock 2810 relative to the outer tube 2006 causes the distal finger 2812 of the lock 2810 to move into engagement with one notch of the plurality of notches 2860 of the pivot assembly 2850 (and see FIG. 55), thereby locking the end effector assembly 4000 in its articulated position.

With continued reference to FIG. 56, the distal movement of the drive beam assembly 4350 and the lock 2810 causes the first leg 2814 of the lock 2810 to move distally past the block 2820, such that the block 2820 is no longer opposing the radially outward biasing force exerted on the first leg 2814, thereby allowing the proximal portion of the first leg 2814 to move or bias (due to a spring bias of first leg 2814 of lock 2810) radially outward. In this position, the foot 2815 of the first leg 2814 contacts the distal ramp 2822 of the block 2820. The radially outward biasing force in connection with the contact between the foot 2815 and the distal ramp 2822 helps maintain the lock 2810 in its distal position.

Retraction of the drive beam assembly 4350 causes the lock 2810 to move proximally to its initial position. Specifically, proximal movement of the drive beam assembly 4350 causes a proximal face 4364b of the tab 4364 to contact the foot 2815 of the first leg 2814 of the lock 2810. After this contact is made, continued proximal movement of the drive beam assembly 4350 moves the first leg 2814 (and the remainder of the lock 2810) proximally until the foot 2815 of the first leg 2814 contacts the proximal wall 2824 of the block 2820. Additionally, during proximal movement of the lock 2810, the distal finger 2812 disengages from the notch of the plurality of notches 2860 of the pivot assembly 2850, and the first leg 2814 of the lock 2810 is urged radially inward due to its engagement with the distal ramp 2822 of the block 2820. When the lock 2810 is in this retracted position, the end effector assembly 4000 is able to articulate relative to the outer tube 2006.

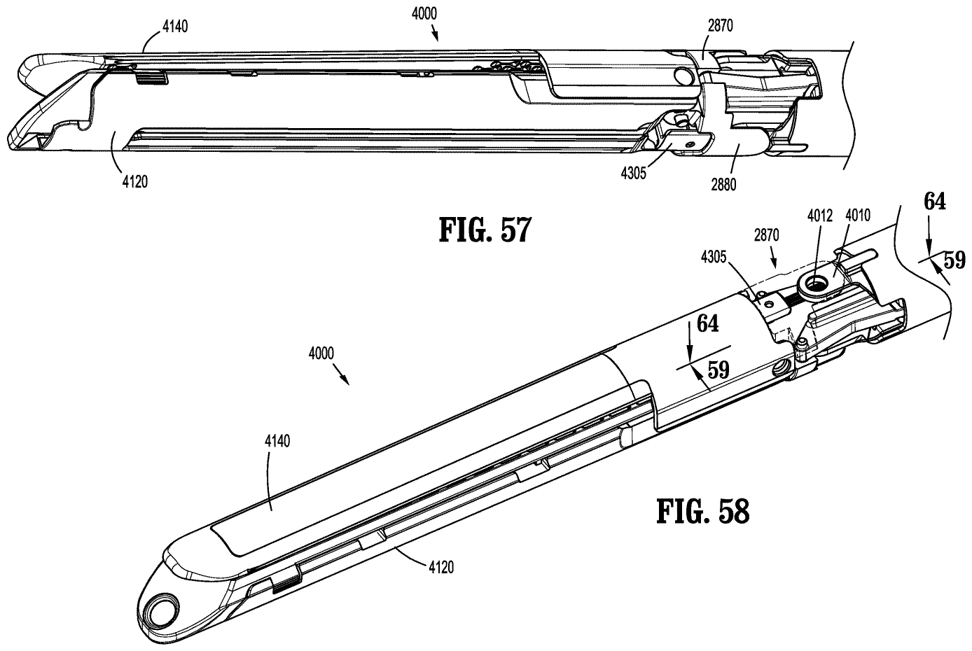

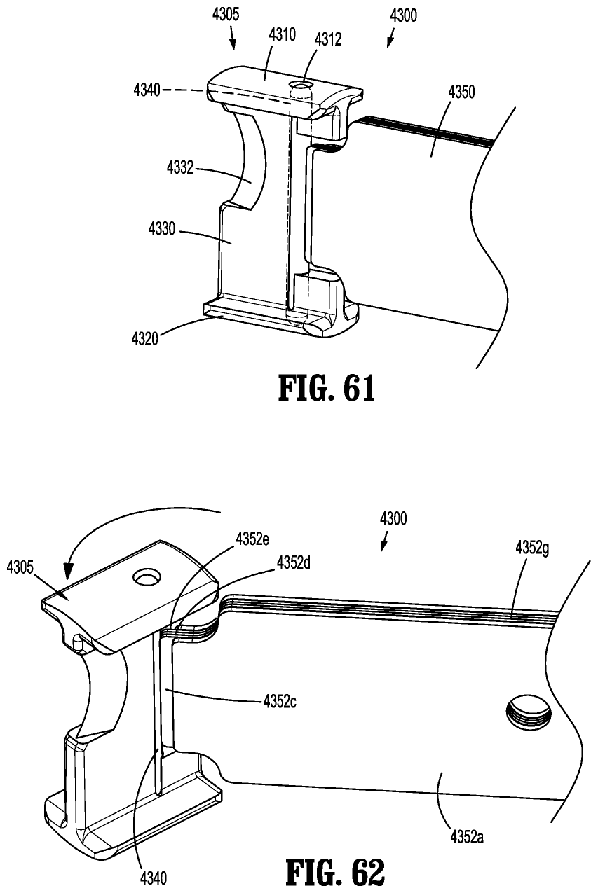

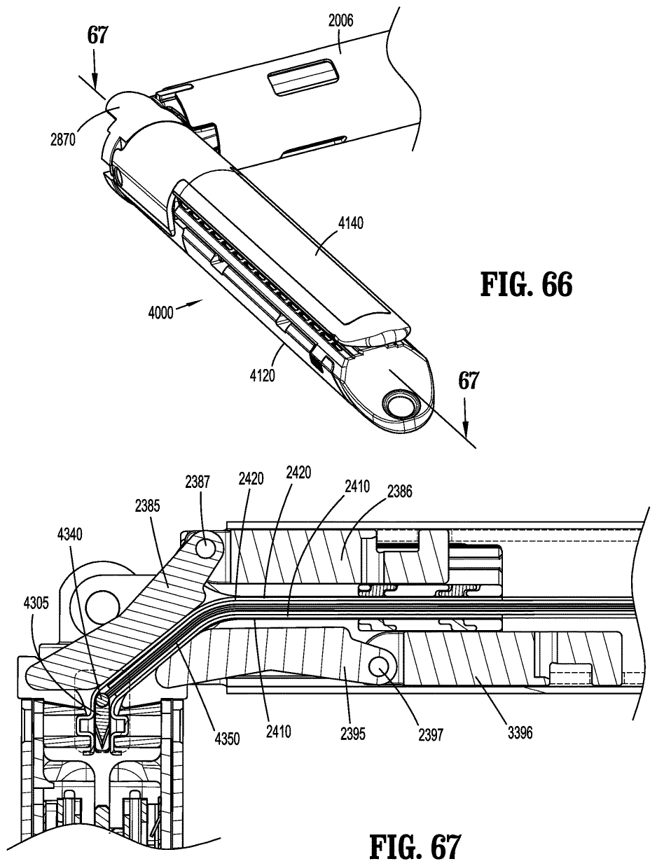

Referring now to FIGS. 30 and 57-67, further details of the end effector assembly 4000 and the I-beam assembly 4300 are shown. The end effector assembly 4000 includes the cartridge assembly 4120 and the anvil assembly 4140, and is configured to engage (e.g., selectively engage) a distal end of the outer tube 2006 via the first tip adapter 4010 and a second tip adapter 4020.

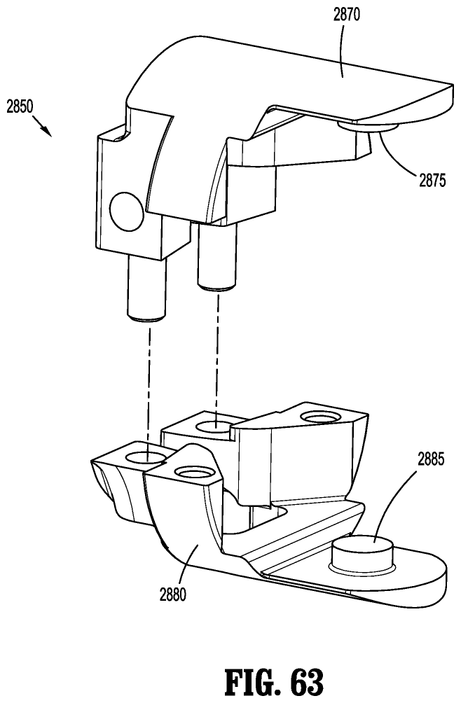

A distal portion of the first tip adapter 4010 defines an aperture 4012 (FIG. 30) configured to engage a first pivot pin 2875 of a first pivot portion 2870 of the pivot assembly 2850 (FIG. 63), and a distal portion of the second tip adapter 4020 defines an aperture 4022 (FIG. 30) configured to engage a second pivot pin 2885 of a second pivot portion 2880 of the pivot assembly 2850 (see FIG. 63). The engagement between the first tip adapter 4010 and the second tip adapter 4020, with the pivot assembly 2850 enables the pivot assembly 2850 (and the cartridge assembly 4120 and the anvil assembly 4140 engaged therewith, for example) to pivot relative to the outer tube 2006.

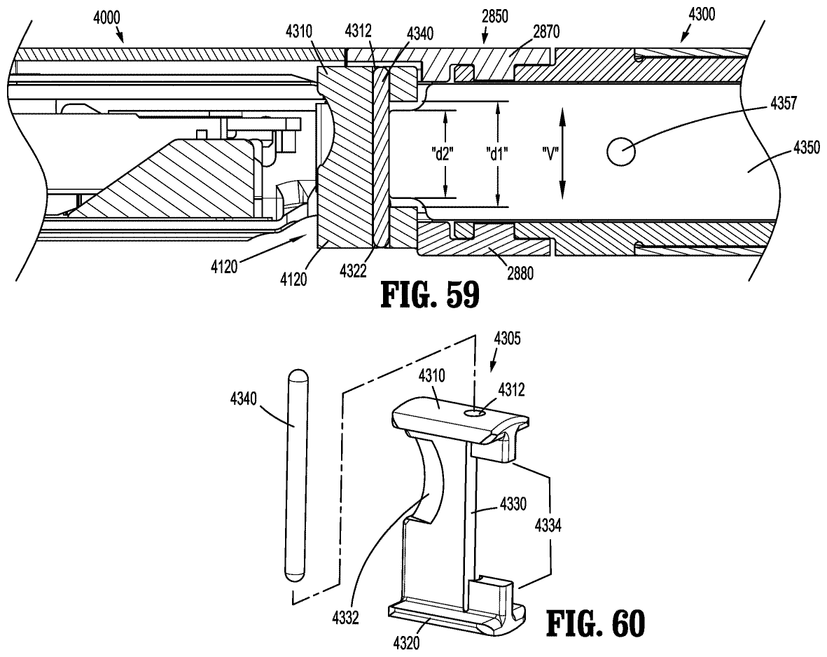

With particular reference to FIGS. 59-60, various components of the I-beam assembly 4300 are shown. The I-beam assembly 4300 includes an I-beam 4305 and the drive beam assembly 4350. The I-beam 4305 includes a first leg 4310, a second leg 4320, a body portion 4330, and a pin 4340. The first leg 4310 is configured to travel at least partially through an anvil channel defined by the anvil assembly 4140, and the second leg 4320 is configured to travel at least partially through a channel defined by the cartridge assembly 4120. The body portion 4330 interconnects the first leg 4310 and the second leg 4320, and includes a knife portion 4332 on its distal edge. A proximal edge of the body portion 4330 defines a recess 4334 therein, which is configured to support distal portions of at least one drive beam of the drive beam assembly 4350. In embodiments, the recess 4334 defines a height “d1”, which is larger than a height “d2” of the portion of drive beam assembly 4350 supported in the recess 4334, thereby allowing the drive beam assembly 4350 to move within the recess 4334 in the directions of arrow “V” (FIG. 59).

The pin 4340 extends through an aperture 4312 within the first leg 4310 and an aperture 4322 within the second leg 4320. Additionally, the pin 4340 is adjacent the recess 4334 of the body portion 4330. The pin 4340 is able to rotate within the apertures 4312, 4322 relative to the first leg 4310, the second leg 4320, and the body portion 4330. Further, the pin 4340 is able to move along the axis of the pin 4340 within the apertures 4312, 4322. That is, a center of the pin 4340 is able to move toward and away from each of the first leg 4310 and the second leg 4320.

As shown in FIG. 59, the pin 4340 of the I-beam 4305 is longitudinally aligned with a portion of the pivot assembly 2850. This location of the I-beam 4305 relative to the pivot assembly 2850 may enable a reduction of the total length of the end effector assembly 4000 (relative to a more distal location of the I-beam 4305), which can be beneficial during certain procedures. The inclusion of the pin 4340 of the I-beam 4305 and/or the manner in which layer(s) of the drive beam assembly 4350 engage the pin 4340 (discussed below) may facilitate or enable the ability of this proximal positioning of the I-beam 4305 by providing a pivotal connection between the I-beam 4305 and the drive beam assembly 4350 (instead of a butt weld, for instance) thereby reducing the amount of stress along the interface between the I-beam 4305 and the layer(s) of the drive beam assembly 4340, and reducing the possibility of cinching at this location during articulation, for instance.

With reference to FIG. 30, the drive beam assembly 4350 includes a plurality of drive beams laminated together. More specifically, in the embodiment illustrated in FIG. 30, the drive beam assembly 4350 includes seven layers or drive beams 4352a-4352g. In disclosed embodiments, drive beams 4352c, 4352d, and 4352e are made of stainless steel, drive beams or layers 4352b and 4352f are made from an adhesive, and drive beams 4352a and 4352g are made from Acrylonitrile butadiene styrene, a common thermoplastic polymer (“ABS”). Drive beams made of other suitable materials are also contemplated by this disclosure.

In the illustrated embodiment, drive beams 4352c, 4352d, and 4352e are the center three beams, drive beam 4352b is disposed outwardly of and in contact with drive beam 4352c, drive beam 4352f is disposed outwardly of and in contact with drive beam 4352e, drive beam 4352a is disposed outwardly of and in contact with drive beam 4352b, and drive beam 4352g is disposed outwardly of and in contact with drive beam 4352f.

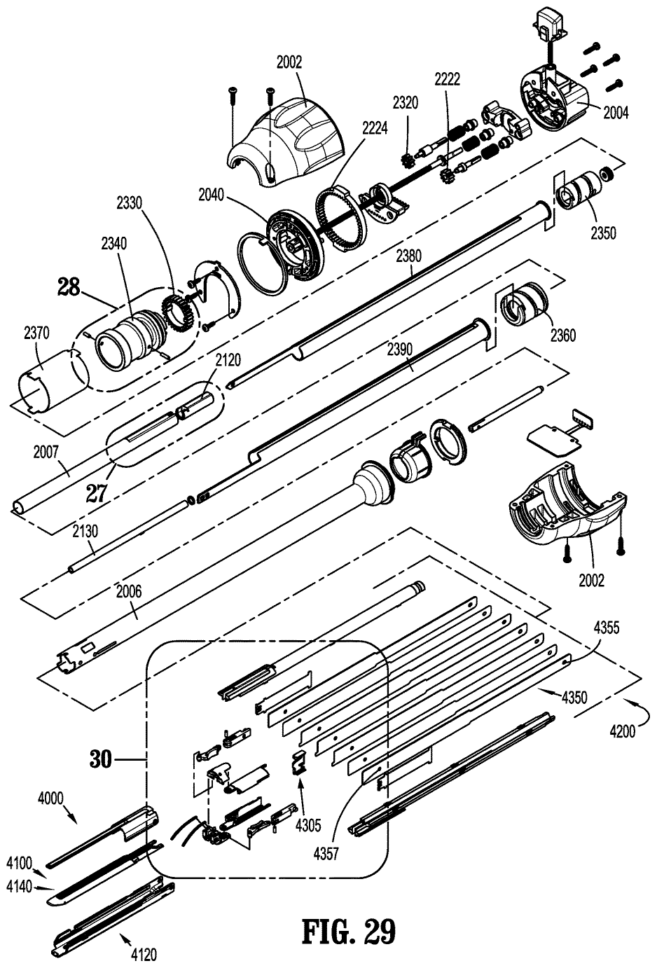

It is envisioned that the drive beams 4352c, 4352d, and 4352e are pinned together and to the firing rod 2130 at a proximal location 4355 (FIG. 29) and are pinned together at a distal location 4357 (FIGS. 29 and 30), and are welded or otherwise secured to the pin 4340 of the I-beam 4305. In embodiments, the pinned connections allow for axial loading of the different layers of the drive beam assembly 4350, and do not constrain the layers transversely. It is further envisioned that the drive beams 4352a and 4352g are secured to the drive beams 4352c and 4352e, respectively, with respective adhesive drive beams 4352b and 4352f.

With reference to FIG. 62, distal ends of drive beams 4352c, 4352d, and 4352e (i.e., the metal layers) are affixed (e.g., welded) to the pin 4340 of the I-beam 4305. The distal end of drive beams 4352a, 4352b, 4352f, and 4352g are free from contact with the pin 4340 and with the remainder of the I-beam 4305 to facilitate ease of movement therebetween. (The drive beams 4352b and 4352f are not shown in FIG. 62 for clarity.) Additionally, as shown in FIG. 40, a firing rod pin 2133 of the firing rod assembly 2130 mechanically couples a distal portion the firing rod assembly 2130 to the drive beam assembly 4350 at the proximal location 4355 of the drive beam assembly 4350. As such, longitudinal movement of the firing rod assembly 2130 results in a corresponding longitudinal movement of the drive beam assembly 4350.

As shown in FIG. 30, the pivot assembly 2850, including the first pivot portion 2870 and the second pivot portion 2880, includes a first jaw spring 2389 and a second jaw spring 2399 coupled thereto. The first jaw spring 2389 extends distally from the pivot assembly 2850, and the second jaw spring 2399 extends distally from the pivot assembly 2850. The outer layers of the drive beam assembly 4350 are configured reduce friction between the drive beam assembly 4350 and the cartridge assembly 4120, the anvil assembly 4140, a first blowout plate 2410, a second blowout plate 2420, the first tip adapter 4010, the second tip adapter 4020, the first pivot portion 2870, and/or the second pivot portion 2880 as the drive beam assembly 4350 is translated longitudinally relative thereto.

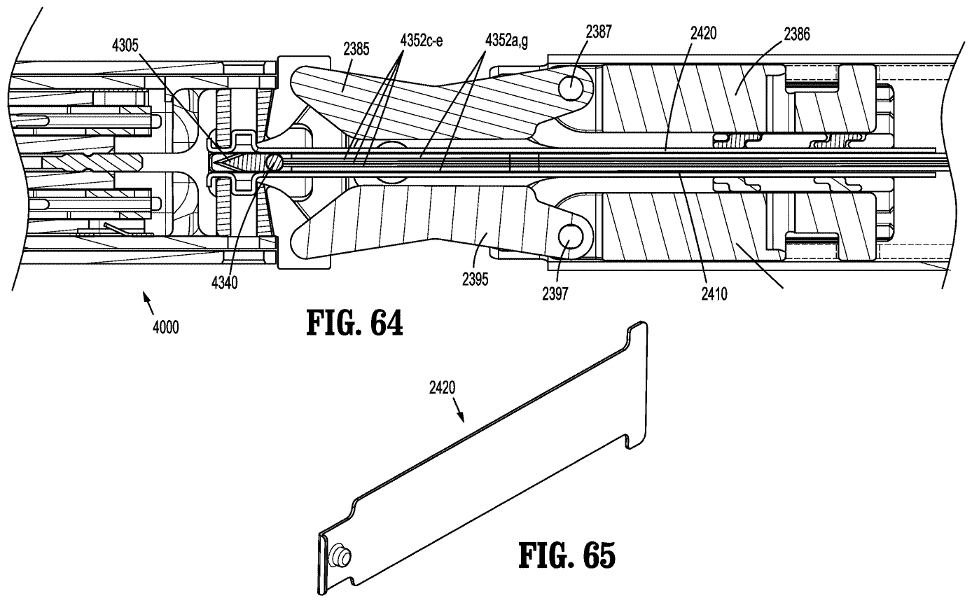

With reference to FIGS. 64-67, a first blowout plate 2410 and a second blowout plate 2420 are shown extending between a distal end of the outer tube 2006 and the proximal end of the end effector assembly 4000. The first blowout plate 2410 is disposed on first lateral side of the drive beam assembly 4350, and the second blowout plate 2420 is disposed on a second lateral side of the drive beam assembly 4350. The first blowout plate 2410 and the second blowout plate 2420 provide lateral support for the drive beam assembly 4350 to help prevent the drive beam assembly 4350 from herniating at the articulation joint (e.g., the first pivot link pin 2387 and the second pivot link pin 2397) during longitudinal movement of the I-beam assembly 4300 while the end effector assembly 4000 is in an articulated position, for instance.

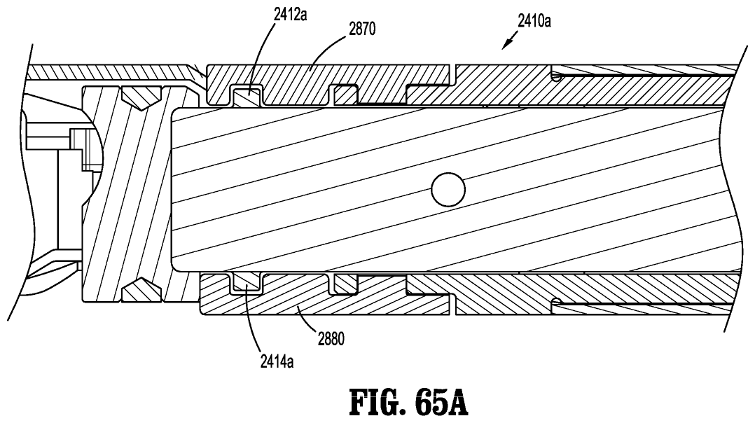

Another embodiment of a blowout plate 2410a (i.e., a first blowout plate and/or a second blowout plate) is shown in FIG. 65A. The blowout plate 2410a of this embodiment is engaged with and anchored to the first pivot portion 2870 and the second pivot portion 2880 utilizing a tabbed connection. More particularly, the blowout plate 2410a includes a first tab 2412a configured to engage a recess in the first pivot portion 2870, and includes a second tab 2424a configured to engage a recess in the second pivot portion 2880.

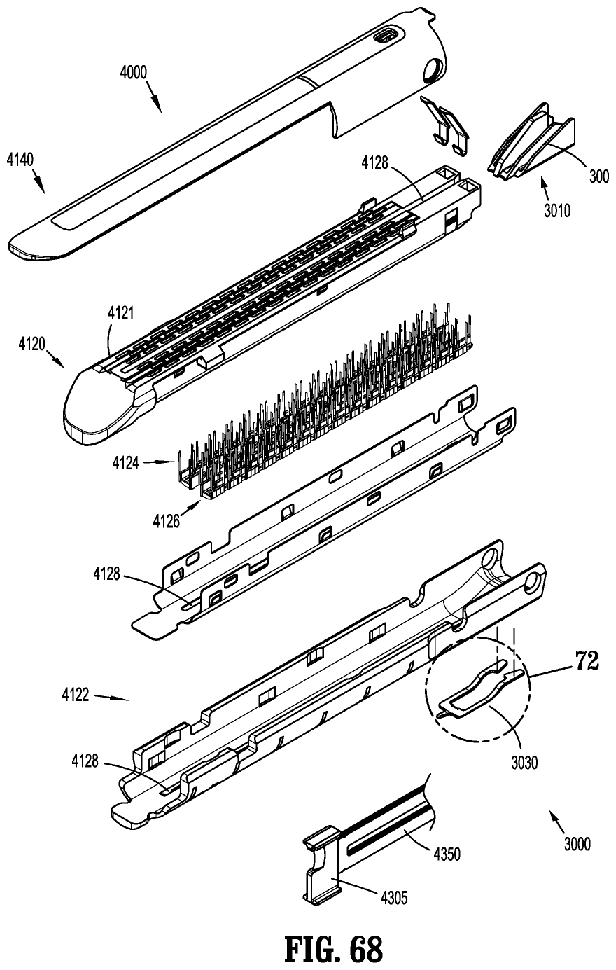

Referring now to FIGS. 68-77, a sled detection mechanism 3000 is shown. The sled detection mechanism 3000 includes the actuation sled 3010, and a sled lockout 3030. Generally, the sled detection mechanism 3000 is configured to permit distal advancement of the I-beam 4305 when the actuation sled 3010 is in a proximal position, and to prevent distal advancement of the I-beam 4305 when the actuation sled 3010 is in a distal position. More specifically, the sled detection mechanism 3000 is configured to prevent the I-beam 4305 from moving distally (e.g., through tissue) after the actuation sled 3010 has travelled distally and already caused fasteners 4124 to be ejected from the cartridge assembly 4120, or when there is no cartridge 4121 engaged with a cartridge channel 4122.

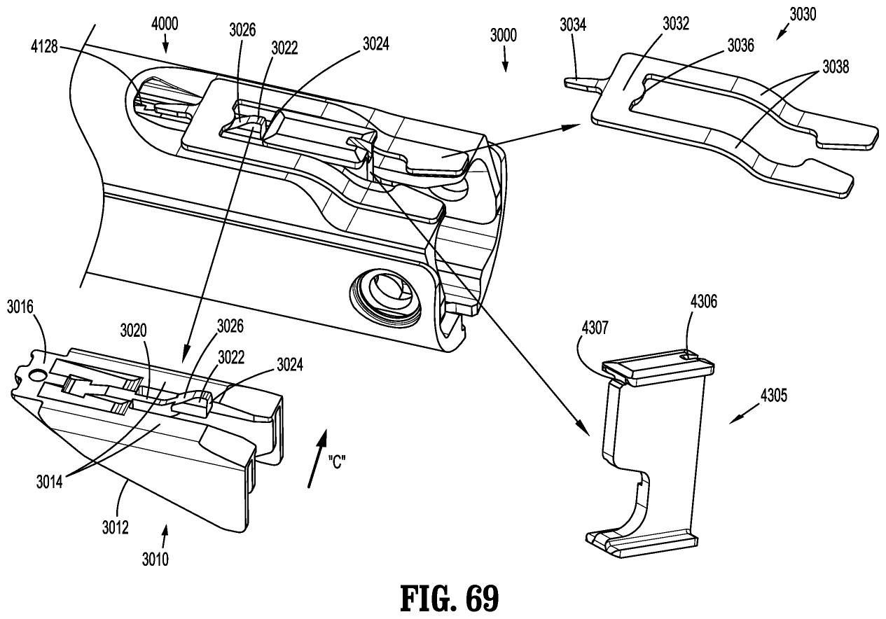

The actuation sled 3010 is disposed at least partially within the cartridge assembly 4120, and adjacent a distal portion of the I-beam 4305. With particular reference to FIG. 69, the actuation sled 3010 includes a wedge portion 3012, a rail 3014, a body portion 3016 and an arm 3020. The wedge portion 3012 is configured to engage staple pushers 4126 to eject fasteners 4124 from the cartridge assembly 4120 (FIG. 68). The rail 3014 is configured to slide along a surface of the cartridge assembly 4120. The body portion 3016 interconnects two portions of the rail 3014. The arm 3020 extends proximally from the body portion 3016 and in between the two portions of the rail 3014. At least a portion of the arm 3020 is configured to travel within a central longitudinal slot 4128 of the cartridge assembly 4120.

The arm 3020 is biased in the general direction of arrow “C” in FIG. 69, which is generally away from the wedge portion 3012 of the actuation sled 3010. A proximal end of the arm 3020 includes a finger 3022. The finger 3022 includes a proximal wall 3024 and a distal ramp 3026.

With continued reference to FIG. 69, the sled lockout 3030 is shown. The sled lockout 3030 is positioned in engagement with the cartridge assembly 4120, and includes a body portion 3032, a distal finger 3034 extending distally from the body portion 3032, a camming tab 3036 extending proximally from the body portion 3032, and a pair of legs 3038 extending proximally from the body portion 3032. The distal finger 3034 and the camming tab 3036 are longitudinally aligned with the central longitudinal slot 4128 of the cartridge assembly 4120, while each leg of the pair of legs 3038 is disposed on opposing lateral sides of the central longitudinal slot 4128.

In use, when the actuation sled 3010 is positioned adjacent the I-beam 4305, as shown in FIG. 70, distal movement of the I-beam 4305 results in a corresponding distal movement of the actuation sled 3010, and allows the I-beam 4305 to travel along and/or within the cartridge assembly 4120. The distal movement of the I-beam 4305 and the actuation sled 3010 results in approximation of the cartridge assembly 4120 and the anvil assembly 4140, and ejection of fasteners 4124 from the cartridge assembly 4120. More particularly, in the position shown in FIGS. 70 and 71, distal translation of the I-beam 4305 causes the actuation sled 3010 to move distally into contact or engagement with the sled lockout 3030. Here, the distal ramp 3026 of the finger 3022 of the arm 3020 of the actuation sled 3010 engages the camming tab 3036 of the sled lockout 3030 and moves or pivots the sled lockout 3030 in the general direction of arrow “D” in FIG. 71 from its first position towards its second position. As shown in FIG. 71, when the sled lockout 3030 is in the second position, the sled lockout 3030 does not physically prevent the actuation sled 3010 or the I-beam 4305 from translating distally past the sled lockout 3030.

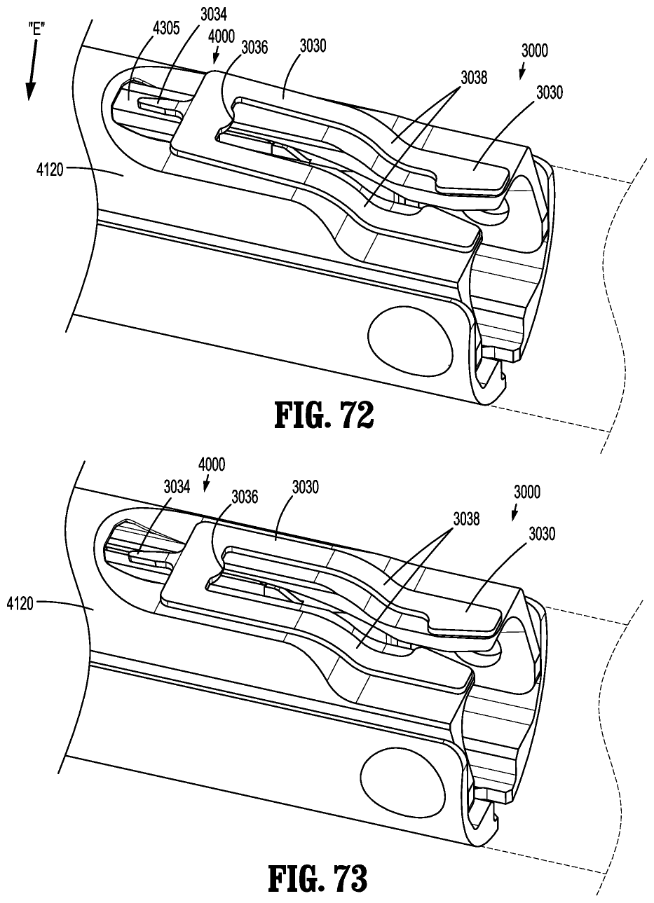

With particular reference to FIG. 72, as the I-beam 4305 distally passes the distal finger 3034 of the sled lockout 3030, the sled lockout 3030 moves or pivots in the general direction of arrow “E” in FIG. 72 towards its first, biased position, and into contact with the I-beam 4305. As the I-beam 4305 continues its distal movement, the distal finger 3034 of the sled lockout 3030 engages and travels along a proximal ramp 4306 of the I-beam 4305. Continued distal movement of the I-beam 4305 results in the I-beam 4305 moving out of engagement with the sled lockout 3030 (FIG. 73), and further results in the I-beam 4305 moving the actuation sled 3010 through the cartridge assembly 4120 to eject fasteners.

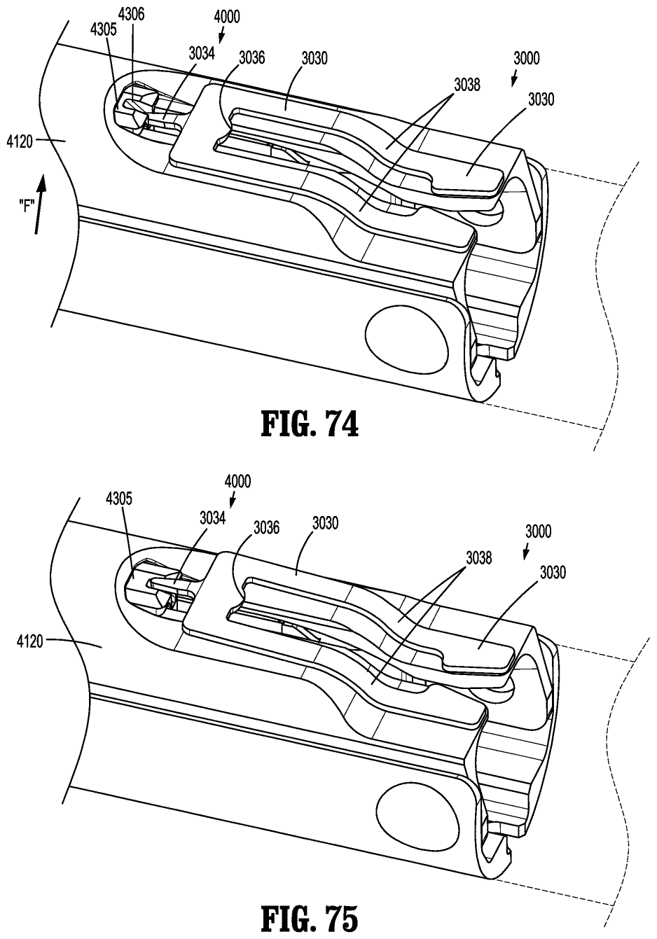

Referring now to FIGS. 74-76, retraction of the I-beam 4305 relative to the cartridge assembly 4120 is shown. After distal translation of the I-beam 4305 and the actuation sled 3010, the I-beam 4305 may be retracted (e.g., for subsequent use with a new cartridge assembly). However, during proximal translation of the I-beam 4305, the actuation sled 3010 remains in its distal position, as retraction of the I-beam 4305 does not result in retraction of the actuation sled 3010.

With particular reference to FIGS. 74 and 75, as the I-beam 4305 moves proximally relative to the cartridge assembly 4120, the proximal ramp 4306 (FIG. 74) of the I-beam 4305 engages the distal finger 3034 of the sled lockout 3030 which pivots the sled lockout 3030 in the general direction of arrow “F” in FIG. 74 toward the second position.

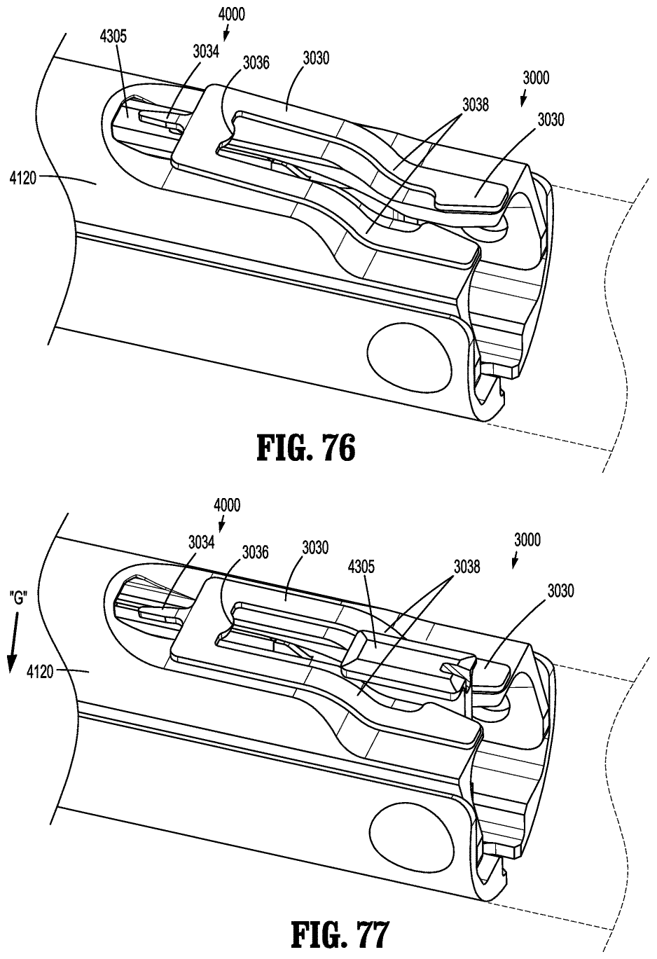

Referring to FIG. 76, when the sled lockout 3030 is in the second position, the I-beam 4305 is able to move proximally of the distal finger 3034 of the sled lockout 3030 and to a position between the pair of legs 3038 of the sled lockout 3030.

As shown in FIG. 77, when the I-beam 4305 is in the proximal position and after the actuation sled 3010 has been translated distally, the sled lockout 3030 moves or pivots in the general direction of arrow “G” towards its first, biased position. Here, the sled lockout 3030 physically prevents or inhibits the I-beam 4305 from being translated distally past the sled lockout 3030. More particularly, distal translation of the I-beam 4305 would cause a distal groove 4307 of the I-beam 4305 (FIG. 69) to engage the camming tab 3036 of the sled lockout 3030. This engagement between the groove 4307 of the I-beam 4305 and the camming tab 3036 does not cause the sled lockout 3030 to pivot; the sled lockout 3030 remains in its initial, biased position and blocks the I-beam 4305 from travelling distally past the camming tab 3036.

Accordingly, the sled detection mechanism 3000 allows the I-beam 4035 to travel distally through the cartridge assembly 4120 while the actuation sled 3010 is travelling distally through the cartridge assembly 4120 to eject fasteners, and prevents the I-beam 4305 from translating distally through the cartridge assembly 4120 after the I-beam 4305 is been retracted to its proximal position when the fasteners have already been ejected from the cartridge assembly 4120.

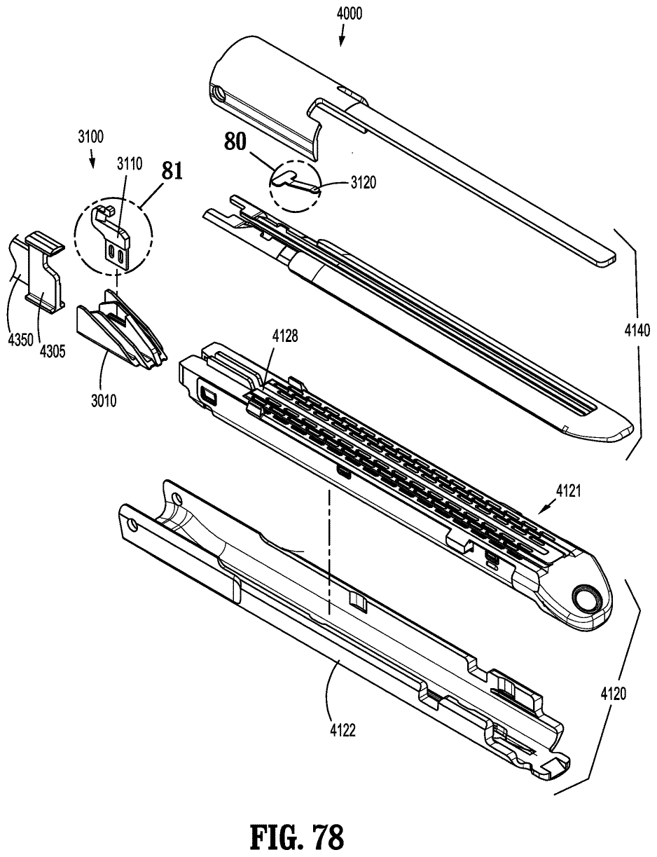

Referring now to FIGS. 78-85, a knife detection mechanism 3100 is shown. The knife detection mechanism 3100 includes a knife 3110 and a lockout spring 3120. Generally, the knife detection mechanism 3100 is configured to permit distal advancement of the I-beam 4305 when the knife 3110 is in a proximal position, and to prevent distal advancement of the I-beam 4305 when the knife 3110 is in a distal position. More specifically, the knife detection mechanism 3100 is configured to prevent the I-beam 4305 from moving distally after the knife 3110 has travelled distally.

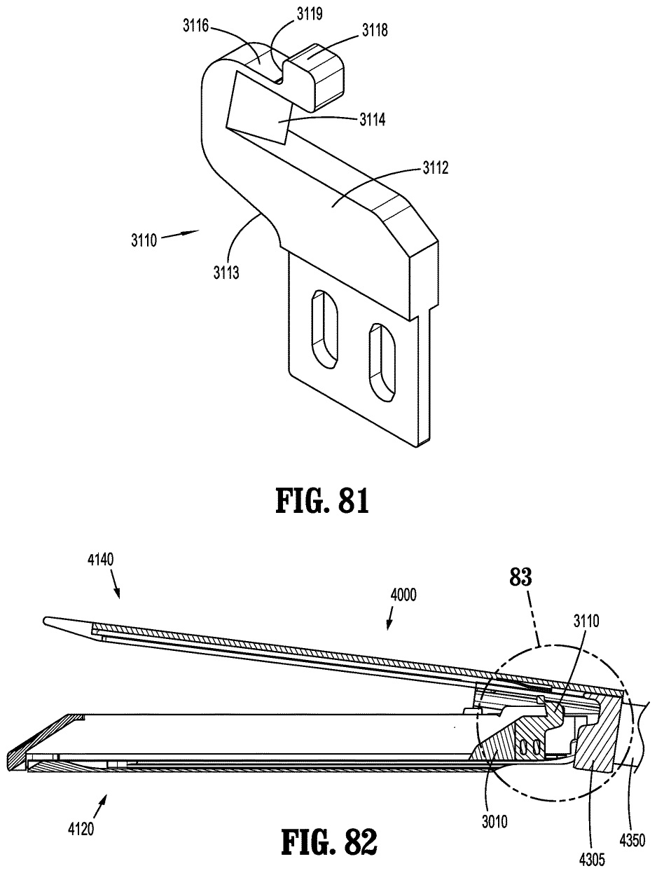

The knife 3110 is disposed at least partially within the cartridge assembly 4120, adjacent a distal portion of the I-beam 4305, and a portion of the knife 3110 is configured to travel within the central longitudinal slot 4128 of the cartridge assembly 4120. With particular reference to FIG. 81, the knife 3110 includes a body portion 3112, a blade 3114, and an arm 3116. A proximal face 3113 of the body portion 3112 is configured to generally mesh with a distal face 4305a of the I-beam 4305 (see FIG. 84). The blade 3114 is configured to cut tissue that is disposed between the cartridge assembly 4120 and the anvil assembly 4140 during distal translation of the knife 3110. The arm 3116 extends from the body portion 3112 adjacent the blade 3114 and includes a finger 3118 on a distal portion thereof.

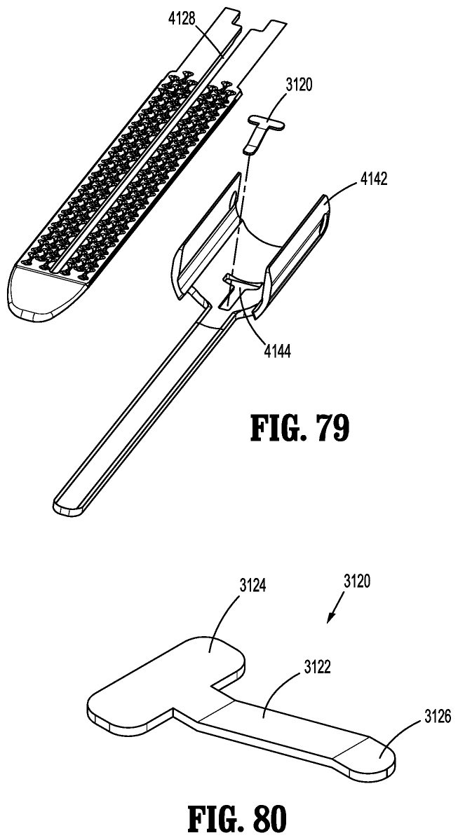

With particular reference to FIGS. 79 and 80, the lockout spring 3120 is shown. The lockout spring 3120 is positioned in engagement with the anvil assembly 4140, and includes a body portion 3122, a leg 3124 extending perpendicularly across a proximal end of the body portion 3122, and a base 3126 positioned adjacent a distal end of the body portion 3122. In embodiments, the base 3126 is secured (e.g., welded) to a carrier 4142 of the anvil assembly 4140. Further, the carrier 4142 of the anvil assembly 4140 defines a T-shaped recess 4144 configured to selectively receive at least a portion of the lockout spring 3120 therein.

The leg 3124 of the lockout spring 3120 is biased away rom the carrier 4142 of the anvil assembly (toward the cartridge assembly 4120) and into a position which physically prevents or hinders distal translation of the I-beam 4305 (when the knife 3110 is not positioned distally adjacent the I-beam 4305).

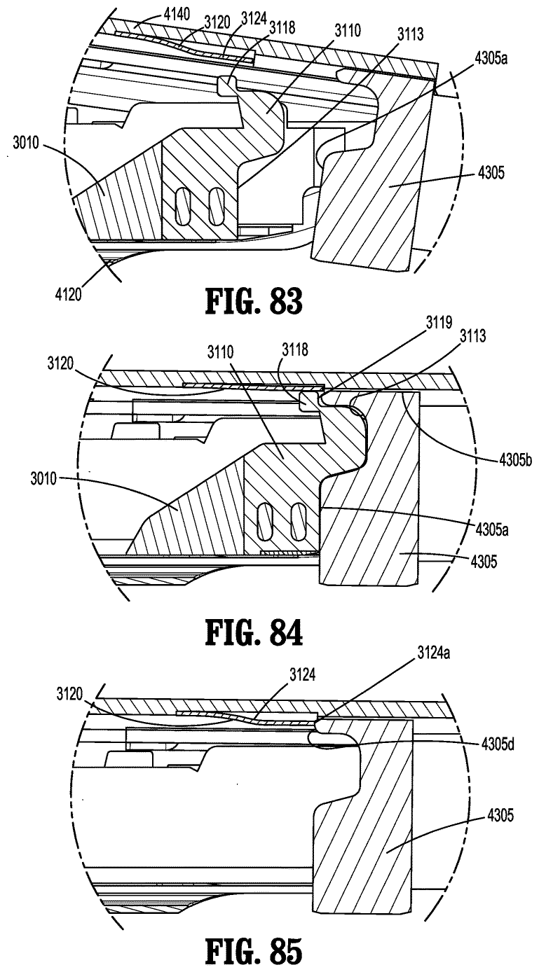

In use, when the knife 3110 is positioned adjacent the I-beam 4305, as shown in FIGS. 83 and 84, approximation of the cartridge assembly 4120 and the anvil assembly 4140 results in the finger 3118 of the knife 3110 engaging the leg 3124 of the lockout spring 3120, which causes the lockout spring 3120 to move from its first, biased position (FIG. 83) to its second position (FIG. 84). When the lockout spring 3120 is in its second position, the leg 3124 of the lockout spring 3120 is no longer in the path of movement of the I-beam 4305, thereby allowing the I-beam 4305 to travel distally relative to the anvil assembly 4140. Here, distal movement of the I-beam 4305 causes the I-beam 4305 to contact a proximal surface 3119 of the finger 3118 of the knife 3110 (FIG. 84). Continued distal movement of the I-beam 4305 results in a corresponding distal movement of the knife 3110, and also results in the leg 3124 of the lockout spring 3120 riding along an upper surface 4305b of the I-beam 4305. Additional distal movement of the knife 3110 may cause the blade 3114 of the knife 3110 to sever tissue between the cartridge assembly 4120 and the anvil assembly 4140, for instance.

After distal translation of the I-beam 4305 and the knife 3110, the I-beam 4305 may be retracted (e.g., for subsequent use with a new cartridge assembly). However, during proximal translation of the I-beam 4305, the knife 3110 remains in its distal position, as retraction of the I-beam 4305 does not result in retraction of the knife 3110.

As the I-beam 4305 moves proximally relative to the anvil assembly 4140, the upper surface 4305b and/or a proximal surface 4305c of the I-beam 4305 engages the lockout spring 3120 which pivots the lockout spring 3120 toward the anvil assembly 4140 and into its second position. When the lockout spring 3120 is in the second position, the I-beam 4305 is able to move proximally of the lockout spring 3120. After the I-beam 4305 moves proximally of the lockout spring 3120, the lockout spring 3120 returns to its initial, biased position (FIG. 85).

As shown in FIG. 85, when the I-beam 4305 is in the proximal position and after the knife 3110 has been translated distally, the lockout spring 3120 physically prevents or hinders the I-beam 4305 from being translated distally past a proximal end of the lockout spring 3120. More particularly, distal translation of the I-beam 4305 would cause a distal surface 4305d of the I-beam 4305 to engage a proximal face 3124a of the leg 3124 of the lockout spring 3120. This engagement between the distal surface 4305d of the I-beam 4305 and the leg 3124 of the lockout spring 3120 does not cause the lockout spring 3120 to pivot; the lockout spring 3120 remains in its initial, biased position and blocks the I-beam 4305 from travelling distally of the lockout spring 3120.

Accordingly, the knife detection mechanism 3100 allows the I-beam 4035 to travel distally through portions the end effector assembly 4000 when the cartridge assembly 4120 and the anvil assembly 4140 are approximated and while the knife 3110 is travelling distally through the anvil assembly 4140, and prevents the I-beam 4305 from translating distally through the anvil assembly 4140 after the I-beam 4305 has been retracted to its proximal position when the knife 3110 has already been advanced through the cartridge assembly 4120 and/or the anvil assembly 4140. Further, the knife detection mechanism 3100 prevents the I-beam 4305 and the knife 3110 from travelling distally through the cartridge assembly 4120 and/or the anvil assembly 4140 when the cartridge assembly 4120 and the anvil assembly 4140 are in the open, non-approximated position.

The knife detection mechanism 3100 may be used separately from or in combination with the sled detection mechanism 3000 discussed herein.

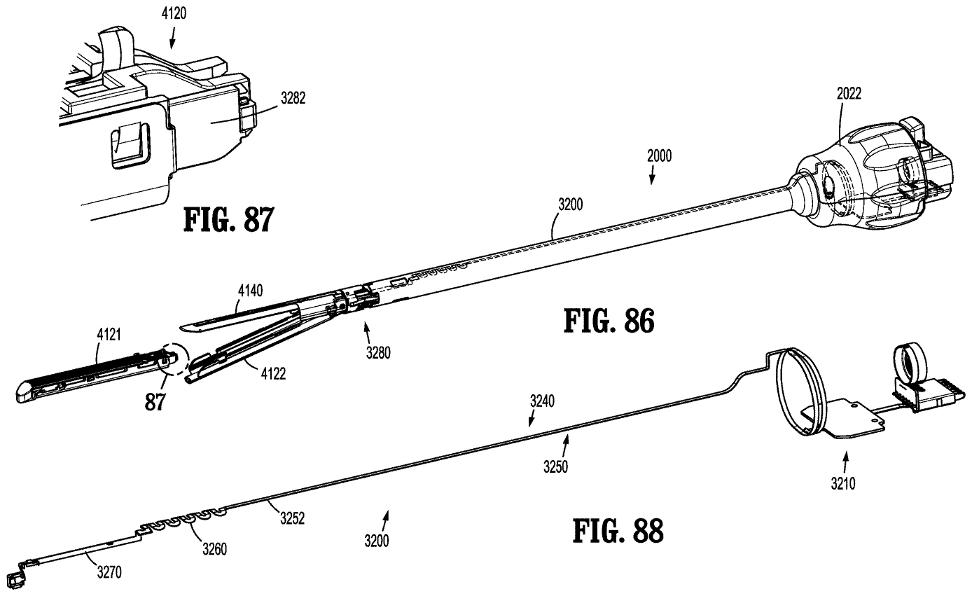

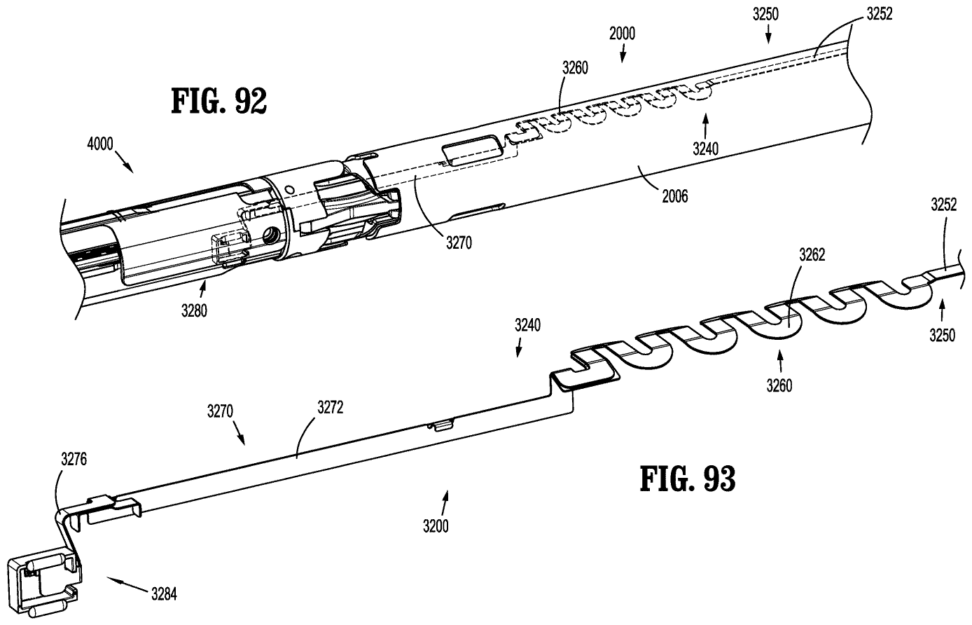

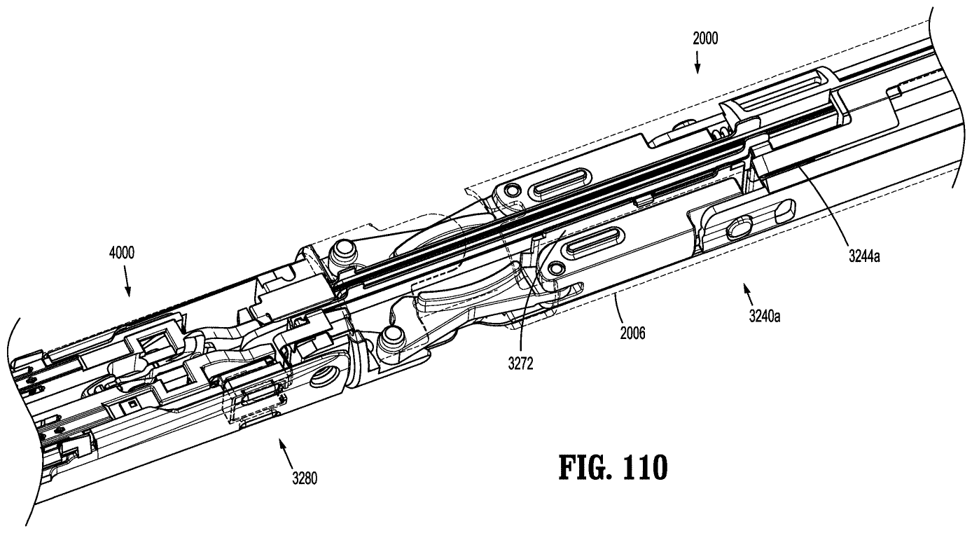

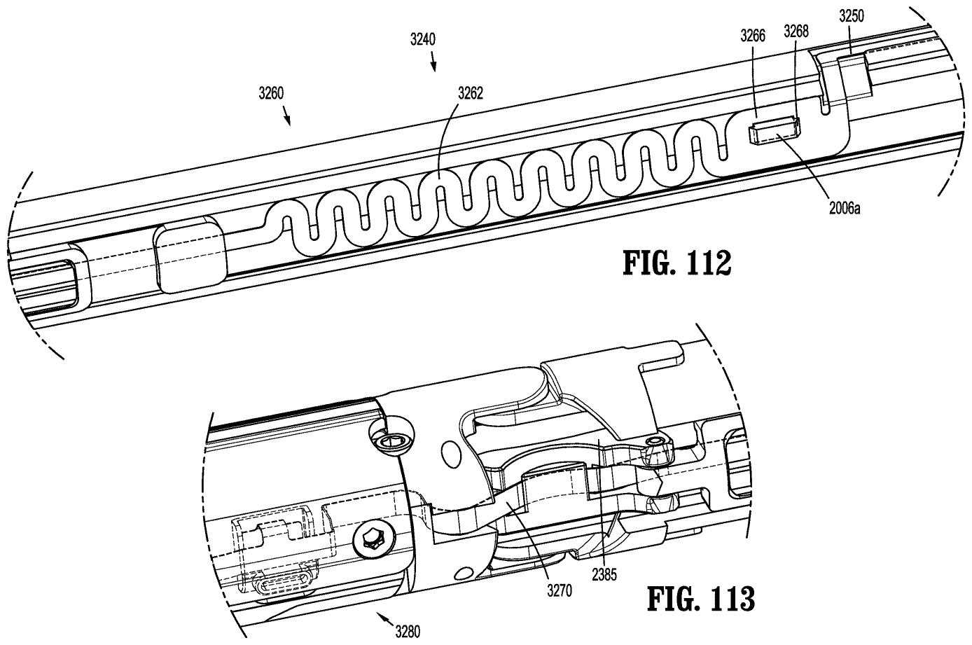

Referring now to FIGS. 86-133, an electrical assembly 3200 of the adapter assembly 2000 is shown. With particular reference to FIGS. 86-88, the electrical assembly 3200 includes a proximal electrical assembly 3210, a flexible cable assembly 3240, and a near field communication (“NFC”) assembly 3280.

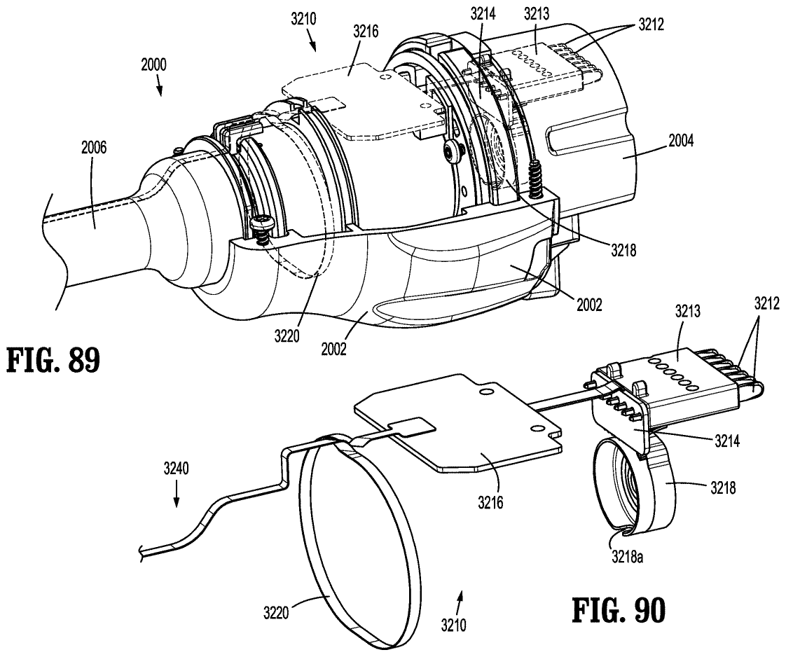

With particular reference to FIGS. 89-91, the proximal electrical assembly 3210 is disposed at least partially within the knob housing 2002, and includes a plurality of electrical contact blades 3212 on a connector 3213, a first circuit board 3214, a second circuit board 3216, a strain gauge 3218, and a service loop 3220. The proximal electrical assembly 3210 is configured to allow for calibration and communication information (e.g., identifying information, life-cycle information, system information, force information) of the adapter assembly 2000 to be relayed to the main controller circuit board 142b of the power-pack core assembly 106 via the electrical receptacle 149 of the power-pack core assembly 106 of the surgical device 100.

The plurality of electrical contact blades 3212 is configured to engage and be in electrical communication with the electrical pass-through connector 66 (FIG. 2) of the surgical device 100 when the adapter assembly 2000 is coupled to the surgical device 100.

The strain gauge 3218 includes a notch 3218a which is configured and adapted to receive a stem (not explicitly shown) of the proximal housing assembly 2004. The engagement between the notch 3218a and the stem functions to restrict rotational movement of the strain gauge 3218 relative to the proximal housing assembly 2004. In embodiments, the strain gauge 3218 provides a closed-loop feedback to a firing/clamping load exhibited by the first proximal drive shaft 2110, based upon which power-pack core assembly 106 sets the speed current limit on the appropriate motor 152, 154, 156.

The service loop 3220 connects a proximal end of the flexible cable assembly 3240 with the second circuit board 3216. The service loop 3220 is configured to maintain electrical communication between the flexible cable assembly 3240 and the second circuit board 3216 during rotation of the outer tube 2006 of the adapter assembly 2000 relative to the core housing 2040 of the adapter assembly 2000.

With reference to FIGS. 86, 88, 92 and 93, the flexible cable assembly 3240 extends between the proximal electrical assembly 3210 and a portion of the NFC assembly 3280, and includes a proximal portion 3250, an intermediate portion 3260, and a distal portion 3270.

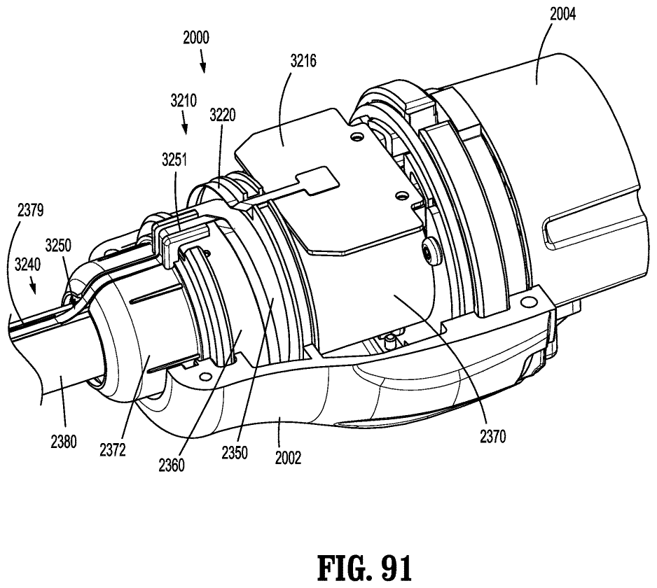

With particular reference to FIGS. 89 and 91, details of portions of the proximal electrical assembly 3210 and the proximal portion 3250 of the flexible cable assembly 3240 are shown in connection with the proximal portion of the adapter assembly 2000 (half of the knob housing 2002 is removed for clarity). As shown, the second circuit board 3216 is positioned radially outward of the isolator tube 2370, the service loop 3220 is positioned radially outward of the proximal cam 2350 and the distal cam 2360, and a proximal end 3251 of the proximal portion 3250 of the flexible cable assembly 3240 extends distally from the service loop 3220, traverses the distal cam bushing 2372, runs along an edge of the first articulation tube 2380 (FIG. 91) (or the second articulation tube 2390), and runs inside a recessed channel of a tube adapter track 2379 (FIG. 91).

With reference to FIGS. 88, 92 and 93, the proximal portion 3250 of the flexible cable assembly 3240 includes a flexible tube cable 3252. The intermediate portion 3260 of the flexible cable assembly 3240 extends distally from the flexible tube cable 3252 of the proximal portion 3250, and includes a flexible cable having a convoluted, coiled, or sinusoidal section 3262. The distal portion 3270 of the flexible cable assembly 3240 includes a flexible cable rigidizer 3272 extending distally from the sinusoidal section 3262. An NFC tag 3282 of the NFC assembly 3280 is coupled to a distal end of the flexible cable rigidizer 3272 with a distal cable 3276 (FIG. 93).

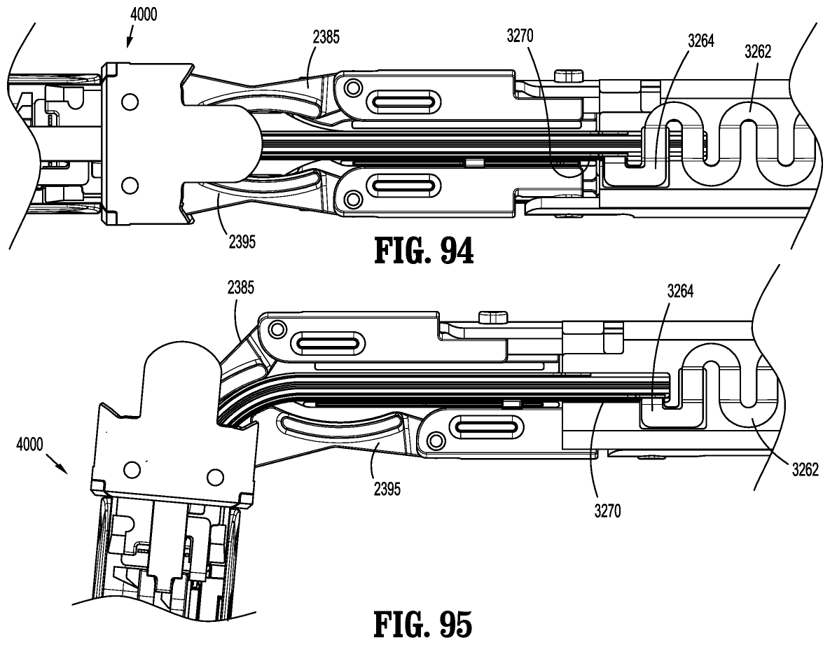

The electrical assembly 3200 is configured to maintain electrical communication between the end effector assembly 4000 of the adapter assembly 2000 and the surgical device 100, during rotation, articulation and/or actuation of the end effector assembly 4000. For example, the sinusoidal section 3262 is configured to apply a proximally-directed force on the distal portion 3270 to help ensure the distal portion 3270 remains under tension, which helps limit the possibility of herniation or bulging of the distal portion 3270 during articulation of the end effector assembly 4000. As shown when comparing FIGS. 94 and 95, a distal end 3264 of the sinusoidal section 3262 moves proximally when the cartridge assembly 4120 moves from a non-articulated position (FIG. 94) to an articulation position (FIG. 95).

The flexible cable rigidizer 3272 is configured to protect itself from damage during movement of components within the outer tube 2006 and/or within the end effector assembly 4000 during use of the surgical device 100. Additionally, the flexible cable rigidizer 3272 is sufficiently rigid to help prevent unintended longitudinal movement thereof during articulation of the end effector assembly 4000, for example.

The NFC assembly 3280 includes an NFC tag 3282 coupled to the cartridge 4121, and an NFC reader 3284 (e.g., an antenna) coupled to a proximal portion of the end effector assembly 4000. The NFC assembly 3280 is configured to relay information about the cartridge 4121 to the adapter assembly 2000 and/or the surgical device 100. For example, components of the NFC assembly 3280 may be used to convey/receive identification/authentication of the cartridge 4121, whether or not the cartridge 4121 is loaded with fasteners and the type of fasteners, the type of cartridge 4121 (e.g., reinforced, unreinforced, length, width), force limits and speed zones associated with the cartridge 4121, number of times the cartridge 4121 (e.g., different cartridges) has been engaged with the adapter assembly 2000, etc.

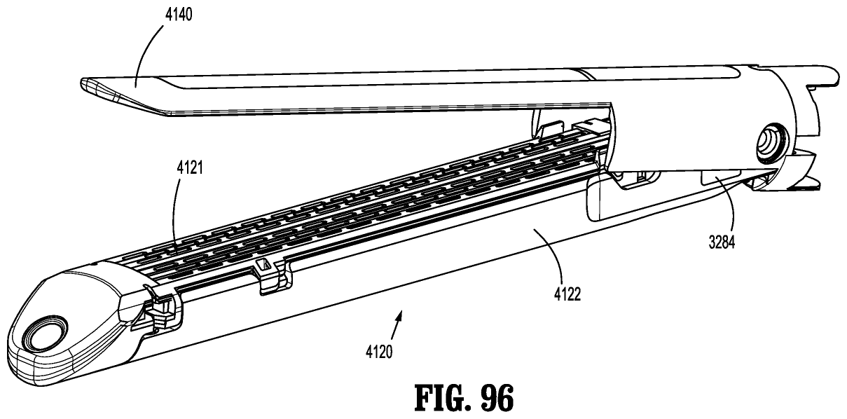

With reference to FIGS. 96-107, various sizes, shapes and locations for the components of the NFC assembly 3280 are shown. While some embodiments include more than one NFC tag 3282 and/or more than one NFC reader 3284, the present disclosure includes more or fewer NFC tags 3282 and/or NFC readers 3284 than the number depicted in a particular embodiment.

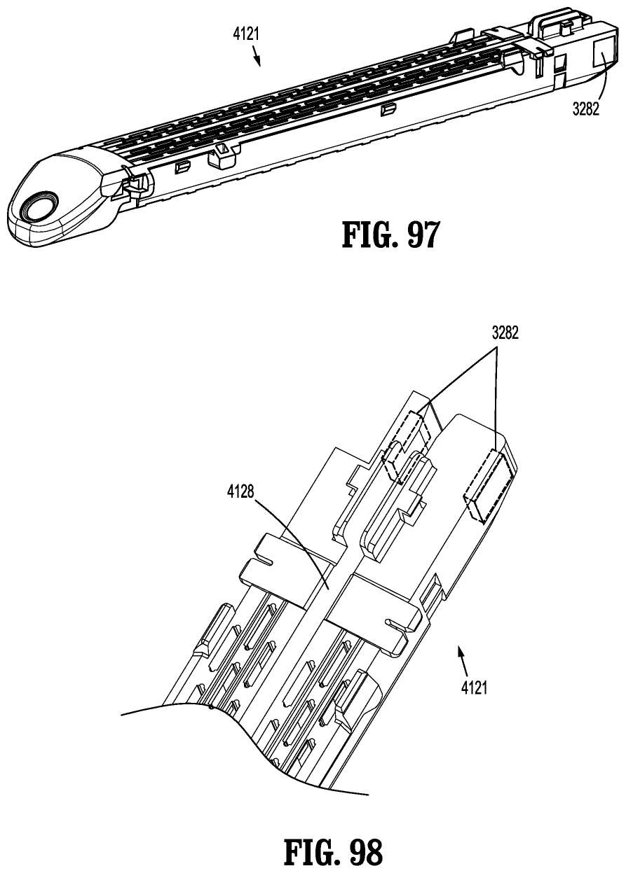

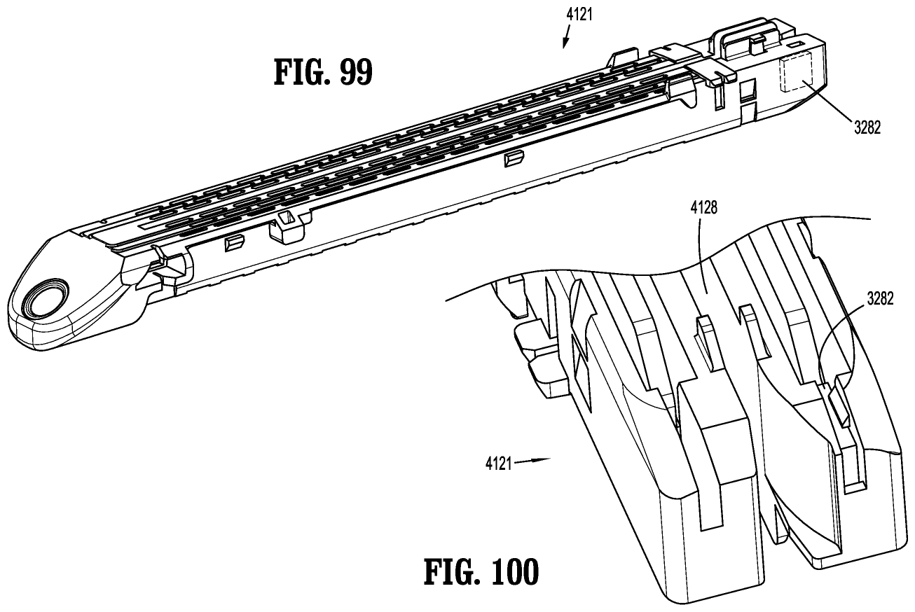

In FIG. 96, the NFC reader 3284 is shown on a lateral side of a proximal portion of the cartridge channel 4122. As shown in FIGS. 97-100, the NFC tag 3282 of this embodiment is disposed on a lateral side of a proximal portion of the cartridge 4121 (FIGS. 97 and 98), adjacent a proximal end of the longitudinal slot 4128 of the cartridge 4121 (FIG. 98), and/or disposed between a lateral side of the proximal portion of the cartridge 4121 and the proximal end of the longitudinal slot 4128 of the cartridge 4121 (FIGS. 99 and 100). Additionally, in these embodiments, the NFC tag 3282 is illustrated as a rectangular prism, but other shapes are envisioned.

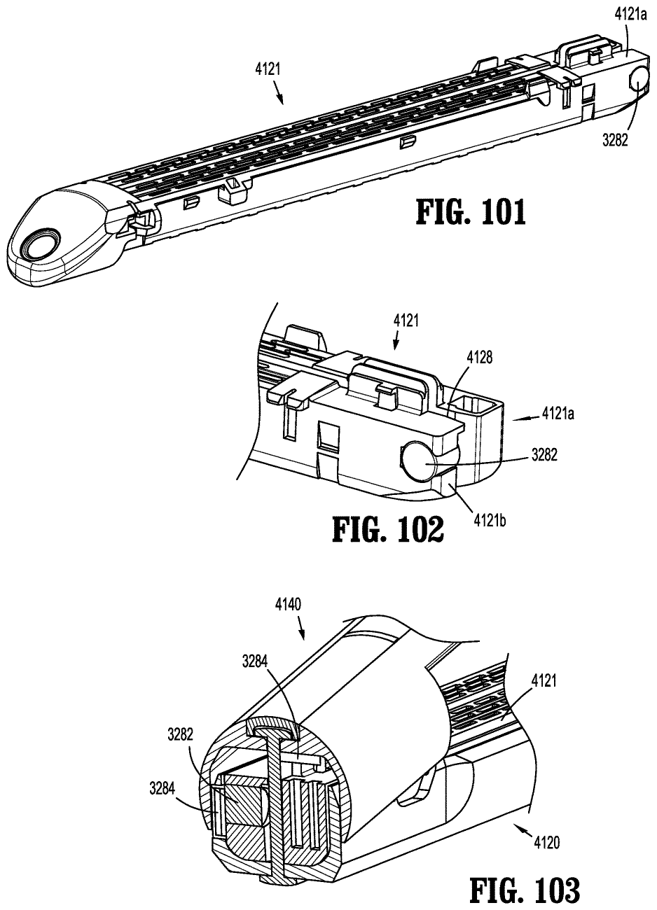

With reference to FIGS. 101-103, the NFC tag 3282 is shown extending laterally at least partially within one side of a fork 4121a at a proximal end of the cartridge 4121. Here, the NFC tag 3282 extends between a lateral side of the proximal portion of the cartridge 4121 and the longitudinal slot 4128 of the cartridge 4121. Additionally, as shown in FIG. 102, the NFC tag 3282 extends proximally beyond a proximal face 4121b of the cartridge 4121. When the NFC tag 3282 is in this position, the NFC tag 3282 can electronically communicate with the NFC reader 3284 when the NFC reader 3284 is positioned in a variety of positions on the cartridge channel 4122, for instance. Further, in the embodiment shown in FIGS. 101-103, the NFC tag 3282 is illustrated as a cylinder, but other shapes are envisioned.

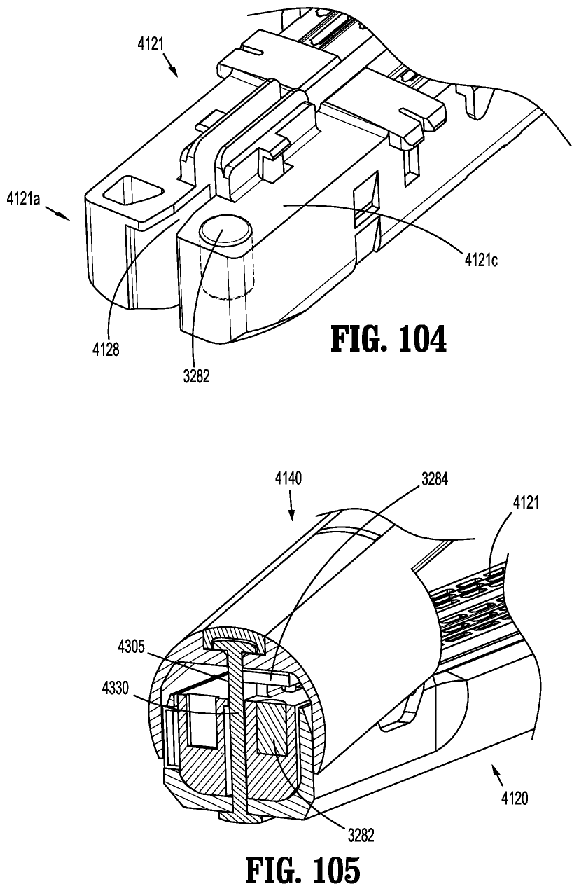

With reference to FIGS. 104 and 105, the NFC tag 3282 is shown extending parallel to the body portion 4330 of the I-beam 4305, and at least partially within one side of the fork 4121a at a proximal end of the cartridge 4121. Here, the NFC tag 3282 extends downwardly from an upper surface 4121c of the cartridge 4121 between a lateral side of the proximal portion of the cartridge 4121 and the longitudinal slot 4128 of the cartridge 4121. When the NFC tag 3282 is in this position, the NFC tag 3282 can electronically communicate with the NFC reader 3284 when the NFC reader 3284 is positioned on the anvil assembly 4140, as shown in FIG. 105, for instance. Further, in the embodiment shown in FIGS. 104 and 105, the NFC tag 3282 is illustrated as a cylinder, but other shapes are envisioned.

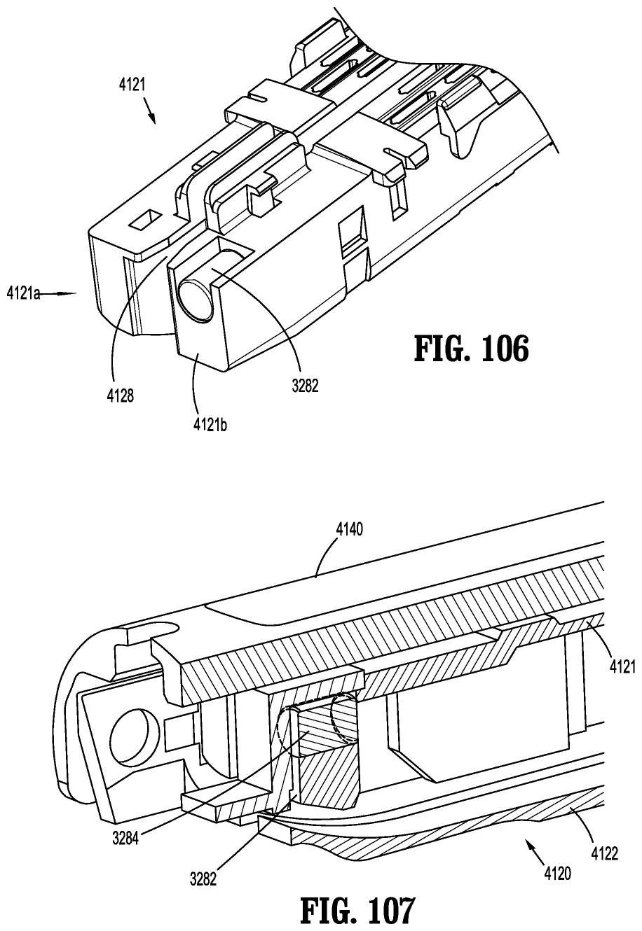

With reference to FIGS. 106 and 107, the NFC tag 3282 is shown adjacent and parallel to the longitudinal slot 4128 of the cartridge 4120, and at least partially within one side of the fork 4121a at a proximal end of the cartridge 4121. Here, the NFC tag 3282 extends distally from the proximal face 4121b of the cartridge 4121 between a lateral side of the proximal portion of the cartridge 4121 and the longitudinal slot 4128 of the cartridge 4121. When the NFC tag 3282 is in this position, the NFC tag 3282 can electronically communicate with the NFC reader 3284 when the NFC reader 3284 is positioned on the cartridge 4121 of the cartridge assembly 4120, as shown in FIG. 107, for instance. Further, in the embodiment shown in FIGS. 106 and 107, the NFC tag 3282 is illustrated as a cylinder, but other shapes are envisioned.

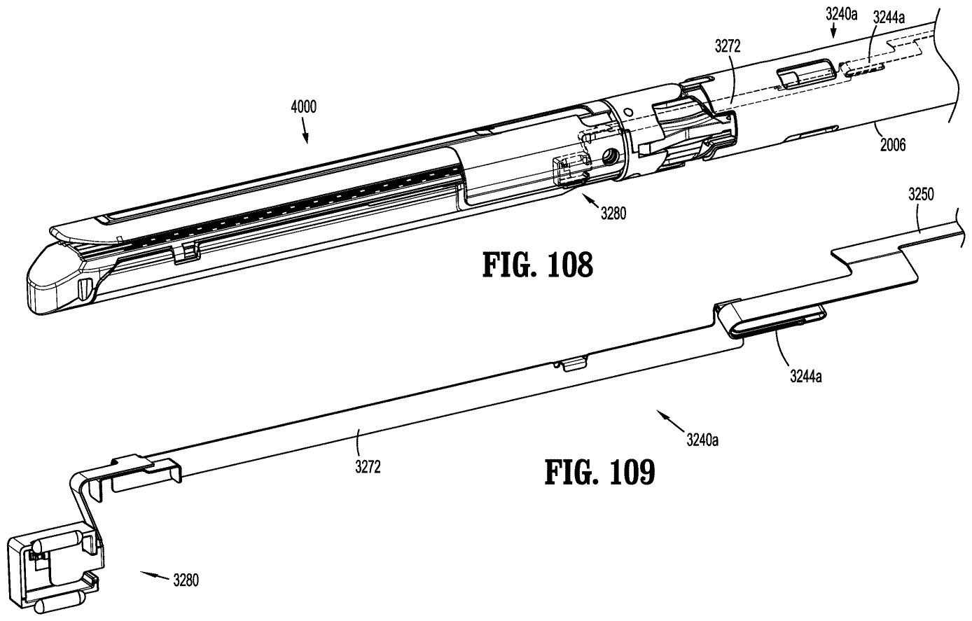

Another embodiment of a flexible cable assembly 3240a is shown in FIGS. 108-110. In this embodiment, the flexible cable assembly 3240a includes a flexible cable loop 3244a instead of the sinusoidal section 3262 of the flexible cable assembly 3240. The flexible cable loop 3244a provides extra length such that the cable rigidizer 3272 can move proximally and distally relative to the proximal portion 3250 of the flexible cable assembly 3240a, and to help prevent herniation of the cable rigidizer 3272 during articulation of the end effector assembly 4000, for instance.

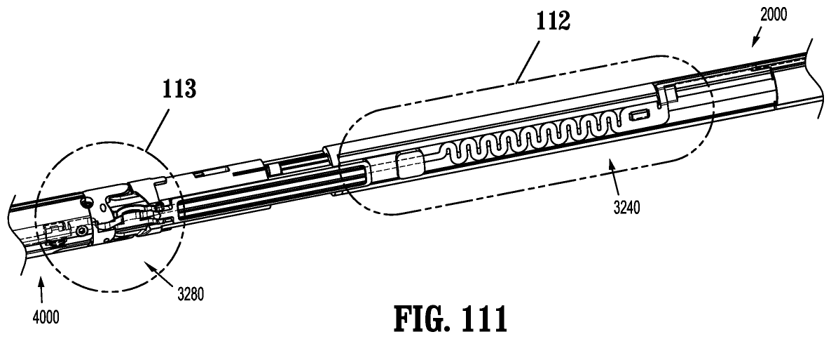

With reference to FIGS. 111-113, further details of the intermediate portion 3260 of the flexible cable assembly 3240 are shown in connection with a disclosed embodiment. Here, a proximal end 3266 of the sinusoidal section 3262 is longitudinally fixed relative to the outer tube 2006, e.g., via engagement with a boss 2006a. This engagement enables the proximal end 3266 of the sinusoidal section 3262 (and thus the proximal portion 3250 of the flexible cable assembly 3240) to remain in its longitudinal position while the rest of the sinusoidal section 3262 is longitudinally expanding or contracting (e.g., during articulation of the end effector assembly 4000). As shown in FIG. 113, the distal portion 3270 of the flexible cable assembly 3240 is routed through the first pivot link 2385 prior to reaching the NFC assembly 3280.

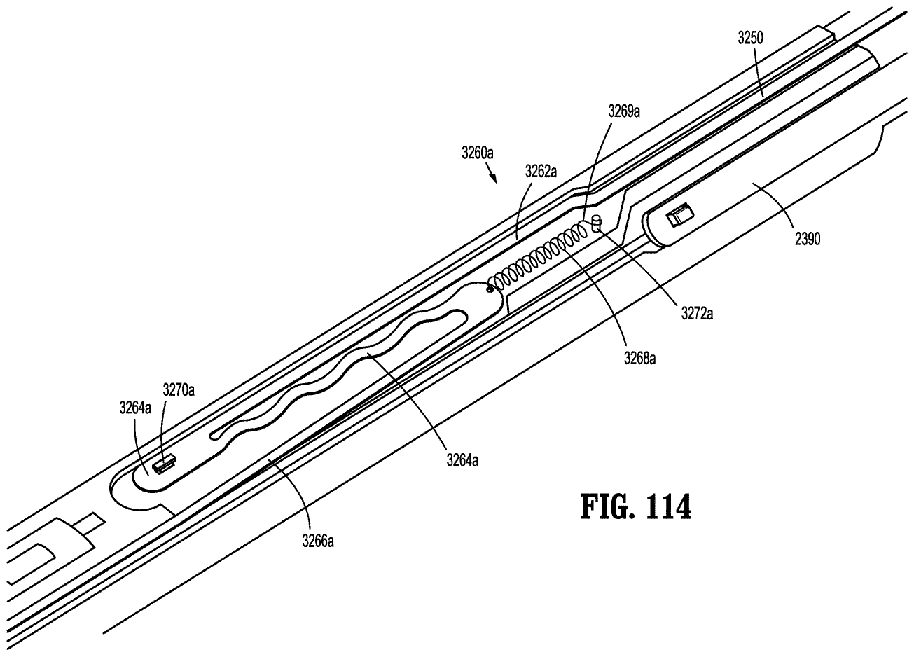

Referring now to FIG. 114, another embodiment of an intermediate portion 3260a of the flexible cable assembly 3240 is shown. Here, the intermediate portion 3260a includes a proximal portion 3262a, an extendable portion 3264a, a distal portion 3266a, and a biasing member 3268a. A distal end 3263a of the proximal portion 3262a is anchored (i.e., fixed from longitudinal movement relative to the outer tube 2006) by a first anchor 3270a. A proximal end 3269a of the biasing member 3268a is anchored (i.e., fixed from longitudinal movement relative to the outer tube 2006) by a second anchor 3272a. The biasing member 3268a biases the extendable portion 3264a and the distal portion 3266a of the intermediate portion 3260a of the flexible cable assembly 3240 proximally. The proximal bias of the distal portion 3266a of the intermediate portion 3260a of the flexible cable assembly 3240 helps ensure the distal portion 3270 of the flexible cable assembly 3240 remains under tension, which helps limit the possibility of herniation or bulging of the distal portion 3270 during articulation of the end effector assembly 4000, for example.

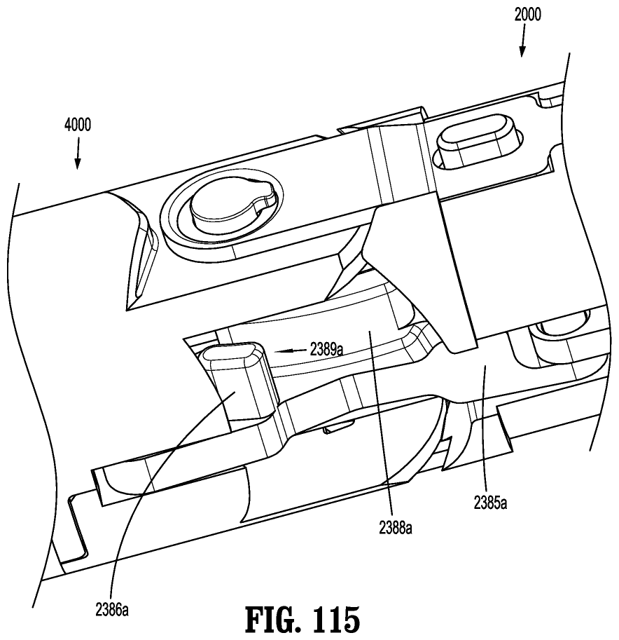

Due to the various parts within the adapter assembly 2000, and to enable the various movements of the end effector assembly 4000, for instance, the electrical assembly 3200 must be routed through the adapter assembly 2000 and the end effector assembly 4000 to ensure such movements remain possible and unimpeded. Disclosed paths, features, and locations for various components of the electrical assembly 3200 are shown in FIGS. 115-129. Additionally, reasonable combinations of these paths, features, and locations for particular elements of the electrical assembly 3200 are also contemplated by this disclosure.

With reference to FIGS. 115-122, various embodiments of the first pivot link 2385 are shown, and each embodiment includes a different feature to help route and/or protect the distal portion 3270 of the flexible cable assembly 3240 (e.g., during articulation of the end effector assembly 4000). While only the first pivot link 2385 is shown and described, the second pivot link 2395 may include the same features and may be a mirror image of the first pivot link 2385.

Initially, FIG. 115 illustrates an embodiment of a first pivot link 2385a including a shield 2386a and a curved wall 2388a. The shield 2386a and the curved wall 2388a define a path 2389a through which the distal portion 3270 of the flexible cable assembly 3240 follows. Here, the shield 2386a helps guide the distal portion 3270 of the flexible cable assembly 3240 and protect the distal portion 3270 of the flexible cable assembly 3240 from becoming damaged during use, for instance.

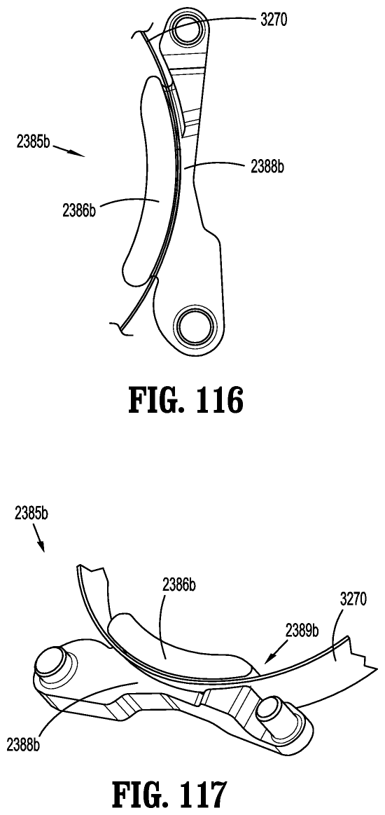

With reference to FIGS. 116 and 117, another embodiment of a first pivot link 2385b is shown. Here, the first pivot link 2385b includes an inner curved wall 2386b and an outer curved wall 2388b, defining a curved path 2389b therebetween. In this embodiment, the inner curved wall 2386b and an outer curved wall 2388b help guide the distal portion 3270 of the flexible cable assembly 3240 and protect the distal portion 3270 of the flexible cable assembly 3240 from becoming damaged during use, for instance.

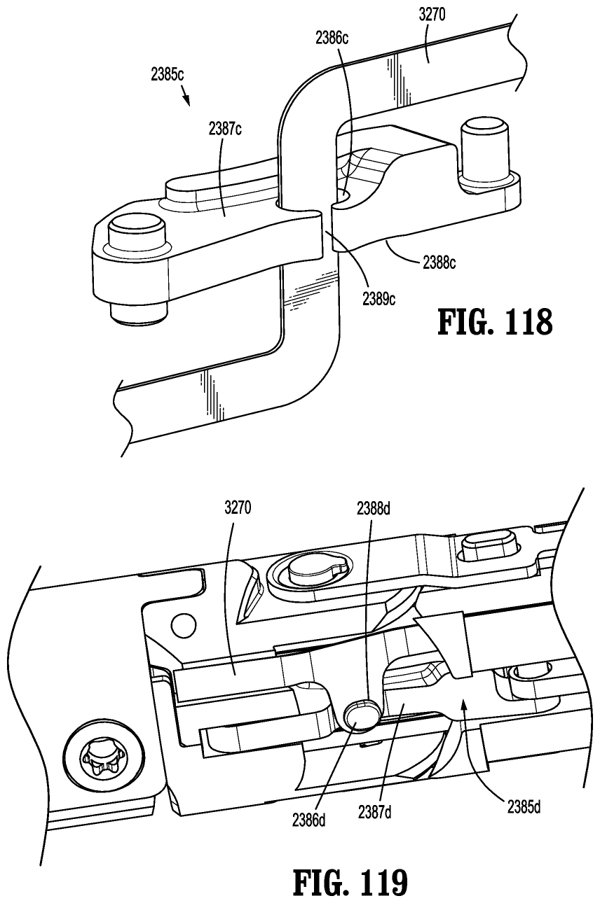

Referring now to FIG. 118, another embodiment of a first pivot link 2385c is shown. Here, the first pivot link 2385c includes an aperture 2386c extending between a first, upper surface 2387c and a second, lower surface 2388c of the first pivot link 2385c. Additionally, a slot 2389c is defined providing access to the aperture 2386c from a lateral side of the first pivot link 2385c. In this embodiment, the distal portion 3270 of the flexible cable assembly 3240 can be inserted through the slot 2389c and into the aperture 2386c, and is able to move relative to the first pivot link 2385c to accommodate articulation of the end effector assembly 4000, for instance.

With reference to FIG. 119, another embodiment of a first pivot link 2385d is shown. Here, the first pivot link 2385d includes a boss 2386d extending outward from an outer wall 2387d. The boss 2386d is configured to receive an aperture 2388d of the distal portion 3270 of the flexible cable assembly 3240. In this embodiment, the distal portion 3270 of the flexible cable assembly 3240 is anchored to the first pivot link 2385d such that the distal portion 3270 of the flexible cable assembly 3240 moves (e.g., translates and bends) with the first pivot link 2385d during articulation of the end effector assembly 4000, for instance.

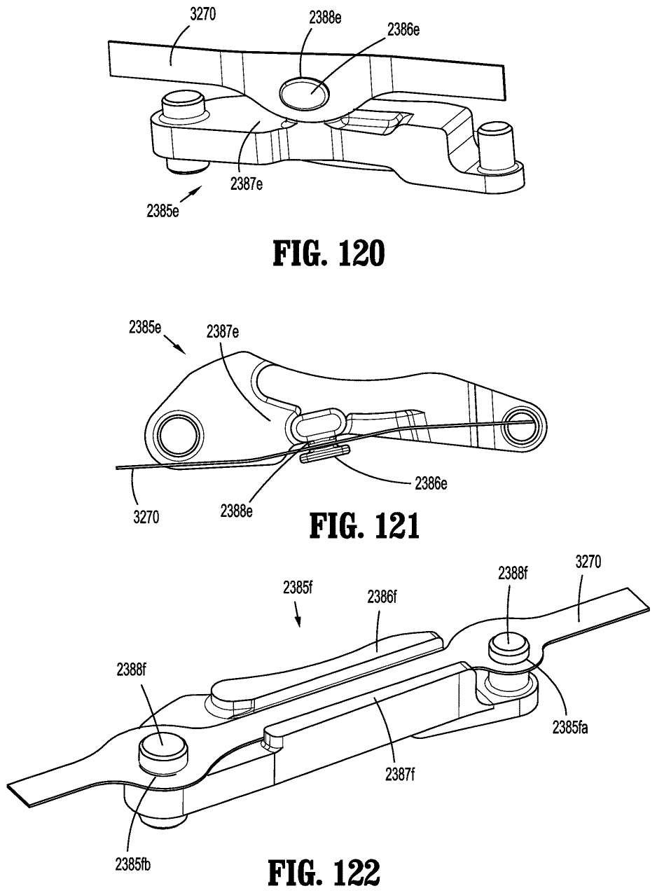

With reference to FIGS. 120 and 121, another embodiment of a first pivot link 2385e is shown. Here, the first pivot link 2385e includes a boss 2386e extending upward and outward from an upper wall 2387e. The boss 2386e is configured to receive an aperture 2388e of the distal portion 3270 of the flexible cable assembly 3240. In this embodiment, the distal portion 3270 of the flexible cable assembly 3240 is anchored to the first pivot link 2385e such that the distal portion 3270 of the flexible cable assembly 3240 moves (e.g., translates and bends) with the first pivot link 2385e during articulation of the end effector assembly 4000, for instance.

With reference to FIG. 122, another embodiment of a first pivot link 2385f is shown. Here, the first pivot link 2385f includes an inner wall 2386f, an outer wall 2387f, a proximal boss 2388f, and a distal boss 2389f. The proximal boss 2388f is configured to receive a proximal aperture 2385fa of the distal portion 3270 of the flexible cable assembly 3240, and the distal boss 2389f is configured to receive a distal aperture 2386fb of the distal portion 3270 of the flexible cable assembly 3240. In this embodiment, the distal portion 3270 of the flexible cable assembly 3240 is anchored to the first pivot link 2385f in two locations such that the distal portion 3270 of the flexible cable assembly 3240 moves (e.g., translates and bends) with the first pivot link 2385f during articulation of the end effector assembly 4000, for instance. Additionally, the distal portion 3270 of the flexible cable assembly 3240 is further prevented from being pinched between moving parts, for example.

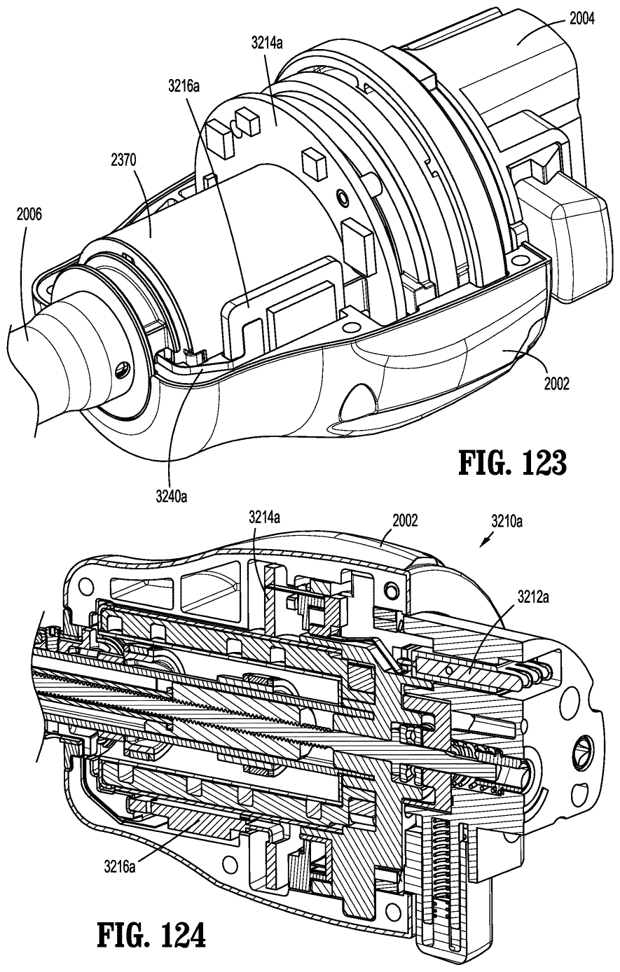

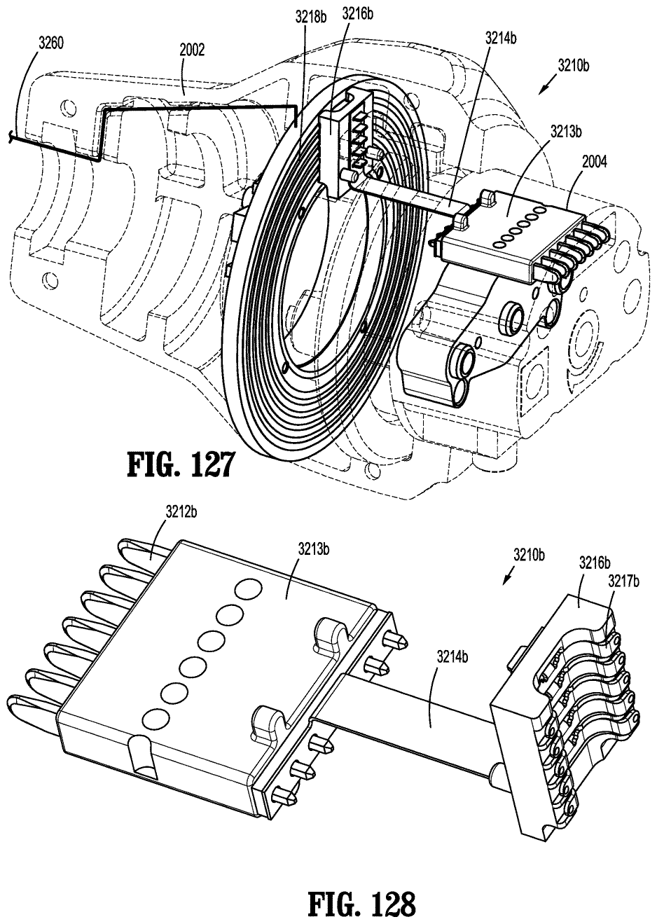

Referring now to FIGS. 123-128, additional embodiments of the proximal electrical assembly 3210 of the electrical assembly 3200 are shown. A first additional embodiment is illustrated in FIGS. 123-126 and is referred to as a proximal electrical assembly 3210a. A second additional embodiment is illustrated in FIGS. 127 and 128 and is referred to as a proximal electrical assembly 3210b.

As shown in FIGS. 123-126, the proximal electrical assembly 3210a includes a plurality of electrical contact blades 3212a on a connector 3213a, a first circuit board 3214a, a second circuit board 3216a, and a service loop 3220a. The plurality of electrical contact blades 3212a is configured to engage and be in electrical communication with the electrical pass-through connector 66 (FIG. 2) of the surgical device 100 when the adapter assembly 2000 is coupled to the surgical device 100. Additionally, a proximal portion of the flexible cable assembly 3240 is shown in FIG. 123. In this embodiment, each of first circuit board 3214a and the second circuit board 3216a is rotationally fixed to the knob housing 2002, such that rotation of the knob housing 2002 (e.g., relative to the isolator tube 2370) results in a corresponding rotation of the first circuit board 3214a and the second circuit board 3216a.

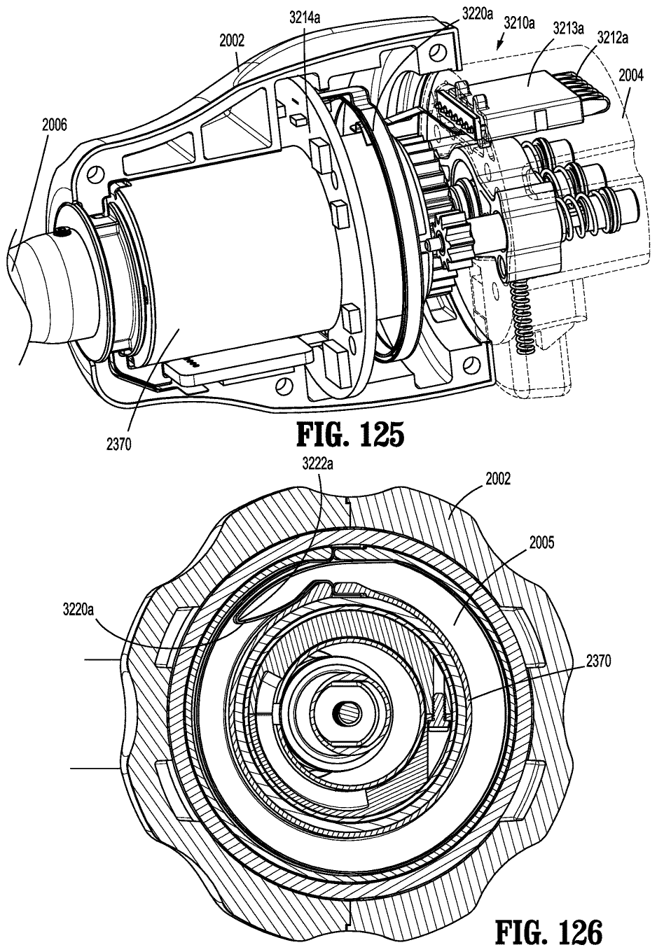

With particular reference to FIGS. 125 and 126, further details of the service loop 3220a are shown. The service loop 3220a interconnects the plurality of electrical contact blades 3212a with the first circuit bord 3214a, and is positioned in a void 2005 between the isolator tube 2370 and the knob housing 2002. The service loop 3220a is of sufficient length to include a bend or overlap portion 3222a which allows or facilitates the service loop 3220a to partially wind and unwind during rotation of the knob housing 2002, and to thereby maintain electrical communication between the plurality of electrical contact blades 3212a and the NFC assembly 3280 during use, for instance.

Referring now to FIGS. 127 and 128, portions of the proximal electrical assembly 3210b are shown. The proximal electrical assembly 3210b includes a plurality of electrical contact blades 3212b on a connector 3213b, a cable (e.g., a flexible cable) 3214b, a slip ring connector 3216b, and a slip ring board 3218b. The plurality of electrical contact blades 3212b is configured to engage and be in electrical communication with the electrical pass-through connector 66 (FIG. 2) of the surgical device 100 when the adapter assembly 2000 is coupled to the surgical device 100. The cable 3214b interconnects the connector 3213b and the slip ring connector 3216b. The slip ring board 3218b is disposed within the knob housing 2002 and is rotationally fixed relative to the knob housing 2002, such that rotation of the knob housing 2002 relative to the slip ring connector 3216b results in a corresponding rotation of the slip ring board 3218b relative to the slip ring connector 3216b.

As shown in FIG. 128, the slip ring connector 3216b includes a plurality of spring-loaded contacts 3217b. The spring-loaded contacts 3217b provide a two-point wiping engagement with the slip ring board 3218b. In embodiments, there is only one point of engagement between the spring-loaded contacts 3217b and the slip ring board 3218b. Accordingly, electrical communication is maintained between the slip ring board 3218b and the slip ring connector 3216b during rotation of the knob 2002, and thus the slip ring board 3218, relative to the slip ring connector 3216b during use of the adapter assembly 2000. Further, the slip ring board 3218b is engaged with the flexible cable assembly 3240 (e.g., via a soldered connection) to provide electrical communication between the slip ring board 3218b and the NFC assembly 3280, for instance.

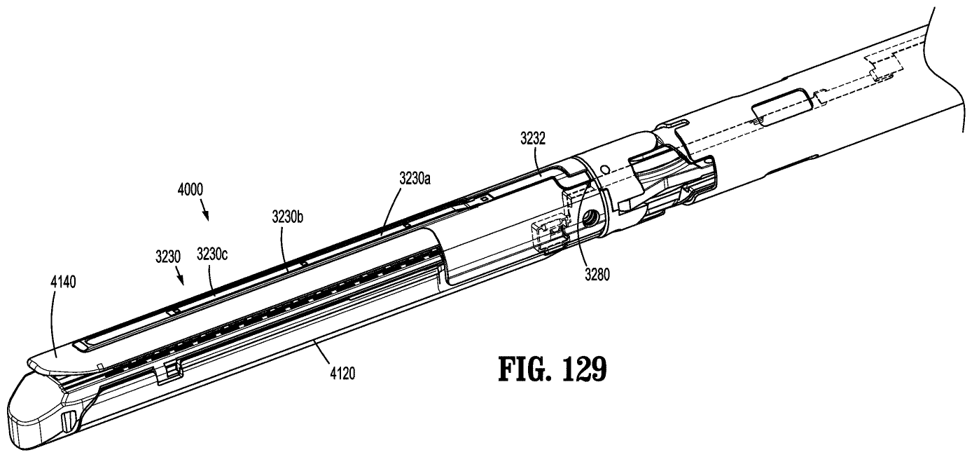

Referring now to FIG. 129, the end effector assembly 4000 is shown including a strain gauge array 3230. A proximal end of the strain gauge array 3230 is affixed (e.g., soldered) to a cable 3232, and the cable 3232 extends from the NFC assembly 3280, thereby electrically coupling the NFC assembly 3280 with the strain gauge array 3230. In embodiments, the strain gauge array 3230 is configured to detect the amount of forces applied to various portions of the end effector assembly 4000, such as along the length of the end effector assembly 4000. In the illustrated embodiment, the strain gauge array 3230 includes three strain gauge elements 3230a, 3230b, and 3230c and is affixed to the anvil assembly 4140. The present disclosure also contemplates the strain gauge array 3230 having more or fewer than three strain gauge elements, and also contemplates the strain gauge array 3230 being mounted on the cartridge assembly 4120, for example.

With reference to FIGS. 130-133, an embodiment of a limited rotation mechanical 3300 is shown. The limited rotation mechanism 3300 is configured to limit the amount of rotation that is enabled between the knob housing 2002 relative to the core housing 2040, which can help protect the portions of the service loop 3220 and/or the flexible cable assembly 3240, for example.

The limited rotation mechanism 3300 includes a pin 3310, a rotation ring 3320, and a clip 3330. The pin 3310 extends radially inwardly from the knob housing 2002. The rotation ring 3320 is positioned at least partially within a track 2041 defined by the core housing 2040, and is rotatable relative to the core housing 2040. The clip 3330 is secured to the core housing 2040 and partially sandwiches the rotation ring 3320 therebetween in a manner that allows rotation of the rotation ring 3320 relative to the core housing 2040.

With continued reference to FIGS. 130-133, the rotation ring 3320 includes a proximally-extending tab 3322. The clip 3330 includes a finger 3332 that is configured to selectively engage the tab 3322 of the rotation ring 3320.

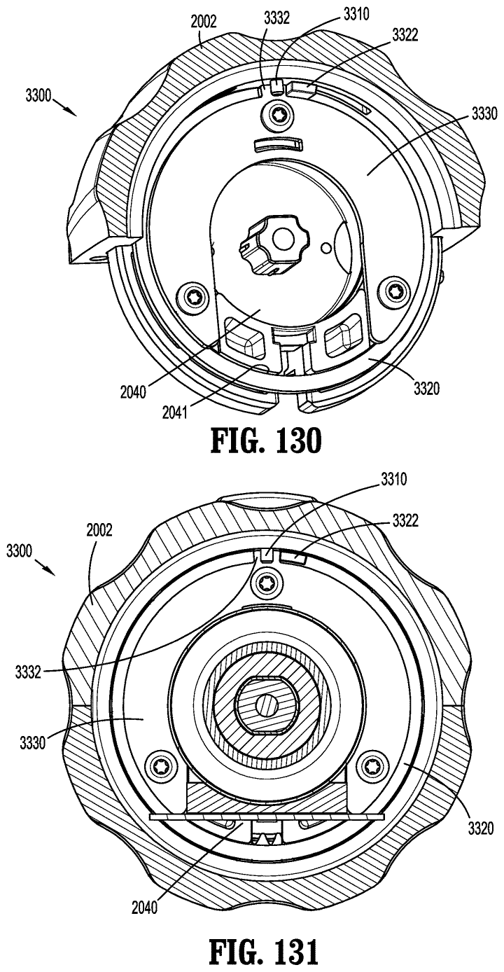

FIGS. 130 and 131 show the knob housing 2002 and the limited rotation mechanism 3300 is an initial position.

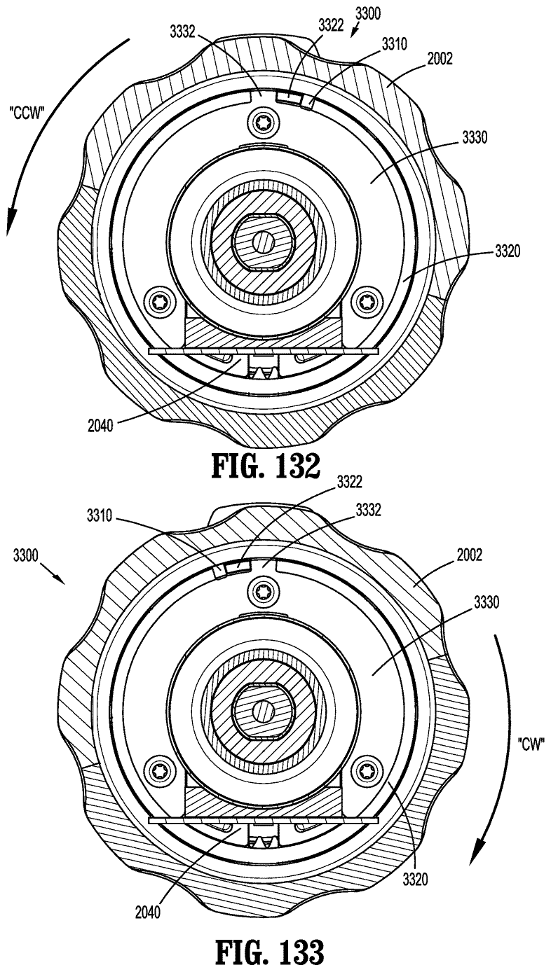

FIG. 132 shows the knob housing 2002 and the limited rotation mechanism 3300 in a second position. Here, the knob housing 2002 and portions of the limited rotation mechanism 3300 have been rotated (e.g., about 340°) in the counter-clockwise “CCW” direction. More particularly, the pin 3310 has been rotated relative to the core housing 2040 until the pin 3310 engaged the tab 3322 of the rotation ring 3320. While the rotation ring 3320 is rotatable relative to the core housing 2040, the engagement between the tab 3322 of the rotation ring 3320 and the finger 3332 of the clip 3330 prevents further rotation of rotation ring 3320 since the clip 3330 is fixed from rotation relative to the core housing 2040.

FIG. 133 shows the knob housing 2002 and the limited rotation mechanism 3300 in a third position. Here, the knob housing 2002 and portions of the limited rotation mechanism 3300 have been rotated (e.g., about 340°) in the clockwise “CW” direction. During this clockwise rotation, the engagement between the pin 3310 and the tab 3322 of the rotation ring 3320 causes rotation of the rotation ring 3320 relative to the core housing 2040. More particularly, the pin 3310 and the rotation ring 3320 have been rotated relative to the core housing 2040 until the tab 3322 of the rotation ring 3320 engaged the finger 3332 of the clip 3330, which prevents further rotation therebetween.

Accordingly, since the knob housing 2002 is rotatable about 340° in both the clockwise and counter-clockwise direction relative to the core housing 2040, the limited rotation mechanism 3300 allows about a 680° rotation of the knob housing 2002 relative to the core housing 2040.

The various embodiments disclosed herein may also be configured to work with robotic surgical systems and what is commonly referred to as “Telesurgery.” Such systems employ various robotic elements to assist the surgeon in the operating theater and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prepare the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

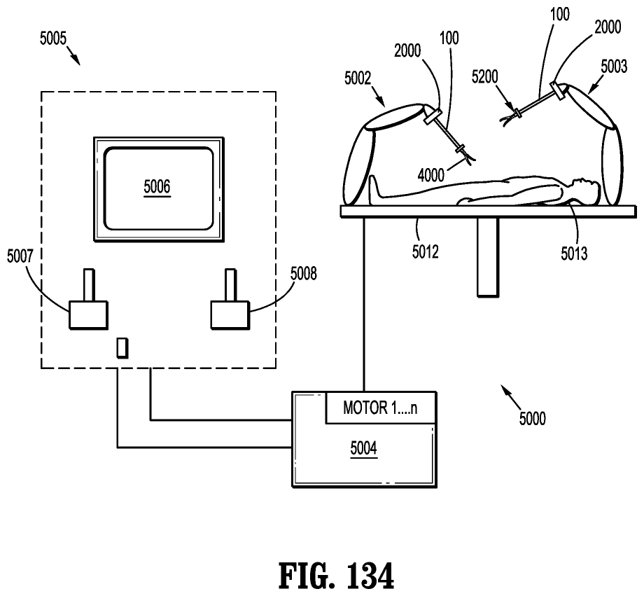

With reference to FIG. 134, a surgical system, such as, for example, a robotic surgical system is shown generally as surgical system 5000 and is usable with the surgical device 100 and/or the adapter assembly 2000, or portions thereof, of the disclosure. The surgical system 5000 generally includes a plurality of robotic arms 5002, 5003, a control device 5004, and an operating console 5005 coupled with the control device 5004. The operating console 5005 includes a display device 5006, which is set up in particular to display three-dimensional images; and manual input devices 5007, 5008, by means of which a person (not shown), for example a surgeon, is able to telemanipulate the robotic arms 5002, 5003 in a first operating mode, as known in principle to a person skilled in the art.

Each of the robotic arms 5002, 5003 is composed of a plurality of members, which are connected through joints. The surgical system 5000 also includes an instrument drive unit 5200 connected to distal ends of each of robotic arms 5002, 5003. The end effector assembly 4000, or portions thereof, may be attached to the instrument drive unit 5200, in accordance with any one of several embodiments disclosed herein, as will be described in greater detail below.

The robotic arms 5002, 5003 may be driven by electric drives (not shown) that are connected to the control device 5004. The control device 5004 (e.g., a computer) is set up to activate the drives, in particular by means of a computer program, in such a way that the robotic arms 5002, 5003, their instrument drive units 5200 and thus the end effector assembly 4000 execute a desired movement according to a movement defined by means of manual input devices 5007, 5008. The control device 5004 may also be set up in such a way that it regulates the movement of the robotic arms 5002, 5003 and/or of the drives.

The surgical system 5000 is configured for use on a patient 5013 lying on a patient table 5012 to be treated in a minimally invasive manner by means of the surgical device 100. The surgical system 5000 may also include more or fewer than two robotic arms 5002, 5003, with any additional robotic arms likewise being connected to the control device 5004 and being telemanipulatable by means of the operating console 5005.

Reference may be made to U.S. Pat. No. 8,828,023, entitled “Medical Workstation,” the entire content of which is incorporated herein by reference, for a detailed discussion of the construction and operation of the surgical system 5000.

It should be understood that the foregoing description is only illustrative of the disclosure. Various alternatives and modifications can be devised by those skilled in the art without departing from the disclosure. Accordingly, this disclosure is intended to embrace all such alternatives, modifications and variances. The embodiments described with reference to the attached drawing figures are presented only to demonstrate certain examples of the disclosure. Other elements, steps, methods and techniques that are insubstantially different from those described above and/or in the appended claims are also intended to be within the scope of the disclosure.

What is claimed is:

1. An articulation assembly for use with a surgical device, the articulation assembly comprising:
a first articulation member disposed in mechanical cooperation with an end effector assembly of the surgical device;
a second articulation member disposed in mechanical cooperation with the end effector assembly of the surgical device;
a first spring-loaded articulation mechanism disposed in mechanical cooperation with the first articulation member and with the second articulation member, the first spring-loaded articulation mechanism including a proximal boss, a distal boss, and a first biasing member extending between the proximal boss and the distal boss; and
a second spring-loaded articulation mechanism disposed in mechanical cooperation with the first articulation member and with the second articulation member, the second spring-loaded articulation mechanism including a proximal boss, a distal boss, and a second biasing member extending between the proximal boss and the distal boss,
wherein distal movement of the first articulation member relative to the second articulation member results in compression of the first biasing member and compression of the second biasing member.

2. The articulation assembly according to claim 1, wherein distal movement of the second articulation member relative to the first articulation member results in compression of the first biasing member and compression of the second biasing member.

3. The articulation assembly according to claim 1, wherein the proximal boss of the first spring-loaded articulation mechanism is longitudinally movable toward and away from the end effector assembly of the surgical device.

4. The articulation assembly according to claim 3, wherein the distal boss of the first spring-loaded articulation mechanism is longitudinally movable toward and away from the end effector assembly of the surgical device.

5. The articulation assembly according to claim 1, wherein the first articulation member defines a first recess having a proximal face and a distal face, and the second articulation member defines a second recess having a proximal face and a distal face.

6. The articulation assembly according to claim 5, wherein the proximal boss of the first spring-loaded articulation mechanism is positioned in contact with at least one of the proximal face of the recess of the first articulation member or the proximal face of the recess of the second articulation member.

7. The articulation assembly according to claim 6, wherein the distal boss of the first spring-loaded articulation mechanism is positioned in contact with at least one of the distal face of the recess of the first articulation member or the distal face of the recess of the second articulation member.

8. The articulation assembly according to claim 5, wherein proximal movement of the first articulation member relative to the second articulation member results in the distal face of the first recess urging the distal boss of the first spring-loaded articulation mechanism proximally.

9. The articulation assembly according to claim 8, wherein distal movement of the second articulation member relative to the first articulation member results in the proximal face of the second recess urging the proximal boss of the first spring-loaded articulation mechanism distally.

10. The articulation assembly according to claim 9, wherein the distal face of the first recess urging the distal boss of the first spring-loaded articulation mechanism proximally is configured to occur at the same time as the proximal face of the second recess urging the proximal boss of the first spring-loaded articulation mechanism distally.

11. An articulation assembly for use with a surgical device, the articulation assembly comprising:
a first articulation member disposed in mechanical cooperation with an end effector assembly of the surgical device;
a second articulation member disposed in mechanical cooperation with the end effector assembly of the surgical device;
a first spring-loaded articulation mechanism disposed in mechanical cooperation with the first articulation member, the first spring-loaded articulation mechanism including a first distal linkage, a first stop member, and a first biasing member, the first biasing member disposed between the first distal linkage and the first stop member; and
a second spring-loaded articulation mechanism disposed in mechanical cooperation with the second articulation member, the second spring-loaded articulation mechanism including a second distal linkage, a second stop member, and a second biasing member, the second biasing member disposed between the second distal linkage and the second stop member,
wherein distal movement of the first articulation member relative to the first stop member results in the first biasing member moving toward an expanded orientation.

12. The articulation assembly according to claim 11, wherein proximal movement of the second articulation member relative to the second stop member results in the second biasing member moving toward a compressed orientation.

13. The articulation assembly according to claim 12, wherein the first biasing member is configured to move toward the expanded orientation at the same time as the second biasing member moves toward the compressed orientation.

14. The articulation assembly according to claim 11, wherein the first distal linkage is directly engaged with the first articulation member.

15. The articulation assembly according to claim 14, wherein the first distal linkage is fixed from longitudinal movement relative to the first articulation member.

16. The articulation assembly according to claim 15, wherein the second distal linkage is directly engaged with the second articulation member.

17. The articulation assembly according to claim 16, wherein the second distal linkage is fixed from longitudinal movement relative to the second articulation member.

18. A surgical device, comprising:
an end effector including a surgical tool;
an outer tube having a distal end, the end effector pivotably connected to the distal end of the outer tube; and
an articulation assembly including:
a first articulation member disposed in mechanical cooperation with the end effector assembly;
a second articulation member disposed in mechanical cooperation with the end effector assembly;
a first spring-loaded articulation mechanism disposed in mechanical cooperation with the first articulation member, the first spring-loaded articulation mechanism including a first distal linkage, a first stop member, and a first biasing member, the first biasing member disposed between the first distal linkage and the first stop member; and
a second spring-loaded articulation mechanism disposed in mechanical cooperation with the second articulation member, the second spring-loaded articulation mechanism including a second distal linkage, a second stop member, and a second biasing member, the second biasing member disposed between the second distal linkage and the second stop member,
wherein distal movement of the first articulation member relative to the first stop member results in the first biasing member moving toward an expanded orientation.

19. The surgical device according to claim 18, wherein proximal movement of the second articulation member relative to the second stop member results in the second biasing member moving toward a compressed orientation.

20. The surgical device according to claim 19, wherein the first biasing member is configured to move toward the expanded orientation at the same time as the second biasing member moves toward the compressed orientation.
